(19) United States
(12) Patent Application Publication
(10) Pub. No.: US 2025/0205238 A1
(43) Pub. Date: Jun. 26, 2025

(54) ANTI-CANCER COMBINATION THERAPY

(71) Applicant: Boehringer Ingelheim International GmbH, Ingelheim am Rhein (DE)

(72) Inventors: Irene WAIZENEGGER, Vienna (AT); Martin AUGSTEN, Vienna (AT); Anke BAUM, Moedling (AT); Daniel GERLACH, Vienna (AT); Jesse LIPP, Vienna (AT); Fabio SAVARESE, Maria Enzersdorf (DE); Hengyu LYU, Shanghai (CN)

(21) Appl. No.: 18/985,253

(22) Filed: Dec. 18, 2024

Related U.S. Application Data
(60) Provisional application No. 63/670,190, filed on Jul. 12, 2024.
(60) Provisional application No. 63/612,548, filed on Dec. 20, 2023.

(51) Int. Cl. A61K 31/519; A61P 35/00

(52) U.S. Cl. CPC A61K 31/519; A61P 35/00

(57) ABSTRACT

The present invention refers to the combination of the HER2 tyrosine kinase zongertinib with a KRAS G12C inhibitor. The combination of the invention is useful for the treatment and/or prevention of oncological and/or hyperproliferative diseases, such as cancer. Accordingly, compounds for use, methods of prevention and/or treatment, uses, pharmaceutical compositions and kits related to the combination are herewith provided.

### FIELD OF THE INVENTION

The present invention refers to the combination of the HER2 tyrosine kinase inhibitor zongertinib, or a pharmaceutically acceptable salt thereof, with a KRAS G12C inhibitor. The combination of the invention is useful for the treatment and/or prevention of oncological and/or hyperproliferative diseases, in particular cancer.

### BACKGROUND OF THE INVENTION

Human epidermal growth factor receptor 2 (HER2; encoded by ERBB2) belongs to the family of ERBB transmembrane receptor tyrosine kinases.

Aberrant activation of HER2 through different mechanisms plays a crucial role in the development and progression of a variety of cancers. For example, more than 20% of human breast cancers overexpresses HER2 resulting from ERBB2 amplification and manifests a historically poor prognosis (Swain, S. M., Shastry, M. & Hamilton, E. Nat. Rev. Drug Discov. 22, 101-126; 2023). Also in 12 to 20% of metastatic gastric, gastroesophageal junction or esophageal adenocarcinoma cases, HER2 is overexpressed (Jorgensen JT, Hersom M. J Cancer; 3, p. 137-144; 2012; Wu H, et al. Tumori; 103(3), p. 249-254; 2017).

In addition to overexpression, HER2 can be aberrantly activated by oncogenic mutations in multiple solid cancers. For instance, HER2 mutations occur in 2-4% of non-small cell lung cancers (NSCLC) and have emerged as important oncogenic drivers (Connell, C. M. & Doherty, G. J. Esmo Open 2, e000279 (2017); Yan, M., et al. Cancer Treat Rev; 40, 770-780 (2014); Stephens, P. et al. Nature 431, 525-526 (2004)). This subset of NSCLC, especially those carrying the most common 12 base pair insertion in ERBB2 exon 20 resulting in the duplication of the amino acids YVMA in HER2 (HER2^{YVMA}), is associated with aggressive disease progression and poor clinical outcomes in response to chemotherapy and immunotherapy.

ERBB signaling can also be aberrantly activated through alterations in the ligands to the receptors of the ERBB family. In this context, fusions of the neuregulin-1 gene (NRG1), although very rare, have been clinically documented (Nagasaka, M. & Ou, S.-H. I. Trends Cancer 8, 242-258 (2022)).

Zongertinib is a potent and selective covalent tyrosine kinase inhibitor of wild type and mutant HER2 that spares wild type epithelial growth factor receptor (EGFR; also referred to as HER1 and encoded by EGFR), another member of the ERBB family. Zongertinib is described in WO 2021/213800 and in Wilding B., et al.; Cancer Discov; 2024; XX; 1-20. It represents a promising treatment option for HER2-dependent cancers.

Downstream of ERBB family receptors, the MAPK signal transducer KRAS is frequently mutated in various types of carcinomas. In NSCLC, the substitution of the residue 12 glycine with cysteine (G12C) is a common KRAS mutation. Despite its high frequency, the development of targeted therapy against KRAS mutated cancer has long been unsatisfactory. A recent exciting development in the treatment of patients with NSCLC is the approval of KRAS^{G12C} inhibitors sotorasib and adagrasib to treat patients with KRAS^{G12C} NSCLC. Treatments with these inhibitors are associated with good response rates but resistance invariably occurs.

Downstream KRAS mutations represent a resistance mechanism to EGFR inhibition (Lopez-Chavez, A., Carter, C. A. & Giaccone, G. Curr. Opin. Investig. drugs (Lond., Engl.: 2000) 10, 1305-14; 2009). On the other hand, co-inhibition of EGFR has been shown to potentiate the effect of inhibiting KRAS^{G12C} and adaptive activation of receptor tyrosine kinases, including EGFR and HER2, has been shown to confer resistance to KRAS inhibition in preclinical studies (Bekaii-Saab, T. S. et al. J. Clin. Oncol.: Off. J. Am. Soc. Clin. Oncol. 41, 4097-4106 (2023)). Preclinical studies have shown that co-targeting an oncogenic KRAS mutant with EGFR more potently inhibits cells dependent on that KRAS mutant than targeting the KRAS mutant alone (McFall, T. et al. Oncol. 6, 86; 2022), leaving room to speculate if selective inhibition of other ERBB members would result in a synergistic combination effect.

Despite all these approaches, there is still a need for improved treatment options for cancer patients. It is therefore an object of the present invention to provide anti-cancer therapies with therapeutic efficacy and safety.

### SUMMARY OF THE INVENTION

In the present invention, it was found that zongertinib inhibits the growth of a KRAS^{G12C} dependent xenograft model synergistically with both KRAS G12C inhibitors sotorasib or adagrasib (Example 1) and that a combination of zongertinib with adagrasib induces persistent tumor shrinkage in PDX models where neither alone can (Examples 2 and 5). Within a certain concentration range, combined, synergistic anti-proliferative activity was observed for zongertinib plus sotorasib or adagrasib in two different KRAS^{Gi2C}-mutant, ERBB2 wild type lung cancer cell lines, where a less pronounced effect was observed for the HER2 tyrosine kinase inhibitor tucatinib in combination with the same KRAS G12C inhibitors (Example 3). The combination of zongertinib with adagrasib was also effective in adagrasib-resistant non-small cell lung cancer models, while also being tolerated (Example 4). Overall, the data presented herein suggest a synergistic effect for combined HER2 and KRAS^{G12C} inhibition. Advantageously, zongertinib enhances the activity of KRAS G12C inhibitors, without causing obvious toxicities. Therefore, the combination of zongertinib with KRAS G12C inhibitors may expand the population of patients who would benefit from KRAS G12C inhibitors. Specifically, it has been shown herein that there is a synergistic effect, defined as a greater than additive benefit, provided by co-administration of zongertinib or a pharmaceutically acceptable salt thereof and a KRAS G12C inhibitor as defined herein in the treatment of cancer. It is believed that, by acting simultaneously, superior inhibition of cancer can be achieved leading to improved cancer treatment outcomes.

The present invention thus provides a combination of zongertinib or a pharmaceutically acceptable salt thereof with a KRAS G12C inhibitor. In embodiments, the combination is administered in an amount to result in a synergistic therapeutic effect. The combination is particularly useful in the treatment and/or prevention of cancer, especially wherein said cancer is KRAS G12C mutant.

According to a first aspect is provided zongertinib or a pharmaceutically acceptable salt thereof for use in the treatment and/or prevention of cancer, wherein zongertinib or the pharmaceutically acceptable salt thereof is administered in combination with a KRAS G12C inhibitor.

Another aspect relates to a KRAS G12C inhibitor for use in the treatment and/or prevention of cancer, wherein the KRAS G12C inhibitor is administered in combination with zongertinib or a pharmaceutically acceptable salt thereof.

Another aspect relates to a method of treating and/or preventing cancer, the method comprising administering to a patient in need thereof: zongertinib or a pharmaceutically acceptable salt thereof and a KRAS G12C inhibitor. In an embodiment, such method comprises administering to a patient in need thereof: a therapeutically effective amount of zongertinib or a pharmaceutically acceptable salt thereof and a therapeutically effective amount (especially a synergistically therapeutically effective amount) of a KRAS G12C inhibitor.

Another aspect relates to a use of zongertinib or a pharmaceutically acceptable salt thereof for the manufacture of a medicament for the treatment and/or prevention of cancer, wherein zongertinib or the pharmaceutically acceptable salt thereof is administered in combination with a KRAS G12C inhibitor.

Another aspect relates to a use of a KRAS G12C inhibitor for the manufacture of a medicament for the treatment and/or prevention of cancer, wherein the KRAS G12C inhibitor is administered in combination with zongertinib or a pharmaceutically acceptable salt thereof.

Another aspect relates to a kit comprising: (i) a first pharmaceutical composition comprising zongertinib or a pharmaceutically acceptable salt thereof and a pharmaceutically acceptable excipient; and (ii) a second pharmaceutical composition comprising a KRAS G12C inhibitor and a pharmaceutically acceptable excipient. In an embodiment, the first pharmaceutical composition comprises a therapeutically effective amount of zongertinib or a pharmaceutically acceptable salt thereof and/or the second pharmaceutical composition comprises a therapeutically effective amount of the KRAS G12C inhibitor.

The definitions of zongertinib, KRAS G12C inhibitor, dosing schedule, combination therapy, additional intervention and cancer provided in the detailed description hereinbelow are applicable to any and all aspects described in the present summary of invention.

In the present invention, any aspect or embodiment referring to a feature (e.g. the identity of the KRAS G12C inhibitor) can be combined with any one or more aspect(s) or embodiment(s) referring to (an)other feature(s) (e.g. the identity of the cancer) to provide further aspects or embodiments of the invention.

### BRIEF DESCRIPTION OF THE FIGURES

FIG. 1. Tumor growth kinetics in NCI-H2122 tumor-bearing mice treated with vehicle, zongertinib, adagrasib or their combination at indicated doses and schedules. Median tumor volumes are plotted over time. Day 1 was the first day, day 84 the last day of the experiment.

FIG. 2. Waterfall plots on day 23 of change of tumor volume relative to day 1 in NCI-H2122 tumor-bearing mice treated with vehicle (not shown), zongertinib (not shown), adagrasib or their combination at indicated doses and schedules. Each bar represents the change of tumor volume on day 23 relative to day 1 for one individual tumor.

FIG. 3. Tumor growth kinetics in NCI-H2122 tumor-bearing mice treated with vehicle, zongertinib, sotorasib or their combination at indicated doses and schedules. Median tumor volumes are plotted over time. Day 1 was the first day, day 38 the last day for calculation of group values and day 64 the last day of the experiment.

FIG. 4. Absolute tumor volume on day 15 in NCI-H2122 tumor-bearing mice treated with vehicle, zongertinib, sotorasib or their combination at indicated doses and schedules. Each symbol represents one individual tumor. The horizontal lines represent the median tumor volume.

FIG. 5. Tumor growth kinetics in ST524 patient derived xenograft (PDX)-bearing mice treated with vehicle, zongertinib, adagrasib or their combination at indicated doses and schedules. Median tumor volumes are plotted over time. Day 1 was the first day, day 28 the last day of the experiment.

FIG. 6. Absolute tumor volume on day 28 in ST524 PDX-bearing mice treated with vehicle, zongertinib, adagrasib or their combination at indicated doses and schedules. Each symbol represents one individual tumor. The horizontal lines represent the median tumor volume.

FIG. 7. Illustration of measurement of BLISS GAP Cell Growth Inhibition (CGI) from measured CGI and expected CGI.

FIG. 8. Cell Growth Inhibition (CGI) (top) and Bliss Gap CGI (bottom) measured for the combination of zongertinib with adagrasib in the NCI-H358 cancer cell line. Only one run is illustrated herein, similar data were obtained in repetitive runs.

FIG. 9. Cell Growth Inhibition (CGI) (top) and Bliss Gap CGI (bottom) measured for the combination of tucatinib with adagrasib in the NCI-H358 cancer cell line. Only one run is illustrated herein, similar data were obtained in repetitive runs.

FIG. 10. Cell Growth Inhibition (CGI) (top) and Bliss Gap CGI (bottom) measured for the combination of zongertinib with sotorasib in the NCI-H358 cancer cell line. Only one run is illustrated herein, similar data were obtained in repetitive runs.

FIG. 11. Cell Growth Inhibition (CGI) (top) and Bliss Gap CGI (bottom) measured for the combination of tucatinib with sotorasib in the NCI-H358 cancer cell line. Only one run is illustrated herein, similar data were obtained in repetitive runs.

FIG. 12. Cell Growth Inhibition (CGI) (top) and Bliss Gap CGI (bottom) measured for the combination of zongertinib with adagrasib in the NCI-H2122 cancer cell line. Only one run is illustrated herein, similar data were obtained in another run.

FIG. 13. Cell Growth Inhibition (CGI) (top) and Bliss Gap CGI (bottom) measured for the combination of tucatinib with adagrasib in the NCI-H2122 cancer cell line. Only one run is illustrated herein, similar data were obtained in another run.

FIG. 14. Cell Growth Inhibition (CGI) (top) and Bliss Gap CGI (bottom) measured for the combination of zongertinib with sotorasib in the NCI-H2122 cancer cell line. Only one run is illustrated herein, similar data were obtained in another run.

FIG. 15. Cell Growth Inhibition (CGI) (top) and Bliss Gap CGI (bottom) measured for the combination of tucatinib with sotorasib in the NCI-H2122 cancer cell line. Only one run is illustrated herein, similar data were obtained in another run.

FIG. 16. Tumor growth kinetics in adagrasib-resistant TC314 tumor-bearing mice treated with vehicle (group A, top curve), adagrasib (group B, middle curve), or the combination of adagrasib and zongertinib (group C, bottom curve) at indicated doses and schedules. Tumor volumes are plotted over time. Gp=group, MRTX849=adagrasib, BI-0631=zongertinib, mpk=mg/kg.

FIG. 17. Body weight changes in adagrasib-resistant TC314 tumor-bearing mice treated with vehicle (group A), adagrasib (group B), or the combination of adagrasib and zongertinib (group C, bottom curve) at indicated doses and schedules. Percentage body weight change is plotted over time. Gp=group, MRTX849=adagrasib, BI-0631=zongertinib, mpk=mg/kg.

FIG. 18. Tumor growth kinetics in adagrasib-resistant TC807R tumor-bearing mice treated with vehicle (group A, top curve with regards to the last point), adagrasib (group B, middle curve with regards to the last point), or the combination of adagrasib and zongertinib (group C, bottom curve) at indicated doses and schedules. Tumor volumes are plotted over time. Gp=group, MRTX849=adagrasib, BI-0631=zongertinib, mpk=mg/kg.

FIG. 19. Body weight changes in adagrasib-resistant TC807R tumor-bearing mice treated with vehicle (group A), adagrasib (group B), or the combination of adagrasib and zongertinib/BI-0631 (group C) at indicated doses and schedules. Percentage body weight change is plotted over time. Gp=group, MRTX849=adagrasib, BI-0631=zongertinib, mpk=mg/kg.

FIG. 20. Tumor growth kinetics in ST4341 patient derived xenograft (PDX)-bearing mice treated with vehicle, zongertinib, adagrasib or their combination at indicated doses and schedules. Median tumor volumes are plotted over time. Day 1 was the first day, day 66 the last day of the experiment.

FIG. 21. Absolute tumor volume on day 28 in ST4341 PDX-bearing mice treated with vehicle, zongertinib, adagrasib or their combination at indicated doses and schedules. Each symbol represents one individual tumor. The horizontal lines represent the median tumor volume.

### DESCRIPTION OF THE INVENTION

### Zongertinib

As used herein, zongertinib refers to the compound as defined below or to a pharmaceutically acceptable salt thereof:

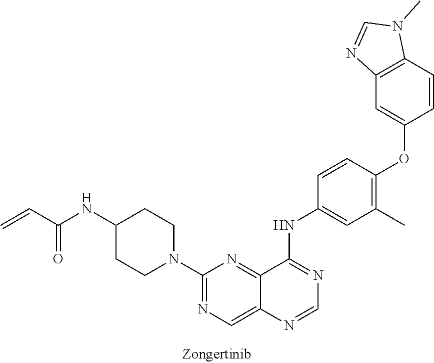 [SMILES: C=CC(=O)NC1CCN(c2ncc3ncnc(Nc4ccc(Oc5ccc6c(c5)ncn6C)c(C)c4)c3n2)CC1]

The IUPAC name of zongertinib is N-{1-[8-({3-methyl-4-[(1-methyl-1H-1,3-benzodiazol-5-yl)oxy]phenyl}amino)-[1,3]diazino[5,4-d]pyrimidin-2-yl]piperidin-4-yl}prop-2-enamide. In case of discrepancy between IUPAC name and depicted formula, the formula shall prevail. WO 2021/213800 discloses zongertinib as example compound I-01 and provides a procedure for its synthesis. Properties of zongertinib and evidence for inhibitory effect on HER2 wild-type and YVMA kinase activity, while sparing EGFR, are also disclosed in WO 2021/213800, which is herein incorporated by reference.

Zongertinib as used herein also encompasses any tautomers, pharmaceutically acceptable salts, solid state forms of the compound, as well as solvates, including hydrates and solvates of pharmaceutically acceptable salts thereof.

In embodiments, zongertinib is a free base. Therefore, in any aspect or embodiment, the expression “zongertinib or a pharmaceutically acceptable salt thereof” can be replaced by “zongertinib”, without a reference to the pharmaceutically acceptable salt thereof. In embodiments, pharmaceutically acceptable salts of zongertinib are used. The term “pharmaceutically acceptable” used herein refers to compounds, materials, compositions and/or dosage forms which are, within the scope of sound medical judgement, suitable for use in contact with the tissues of human beings without excessive toxicity, irritation, allergic response, or other problem or complication, and commensurate with a reasonable benefit/risk ratio.

As used herein, “pharmaceutically acceptable salts” of zongertinib refers to zongertinib wherein the compound is modified by making acid or base salts thereof. The term pharmaceutically acceptable salts as used herein generally includes both acid and base addition salts. Pharmaceutically acceptable acid addition salts refer to those salts which retain the biological effectiveness and properties of the free base and which are not biologically or otherwise undesirable, formed with inorganic acids or organic acids. Pharmaceutically acceptable base addition salts include salts derived from inorganic bases or organic nontoxic bases. Examples of pharmaceutically acceptable salts include, but are not limited to, mineral or organic acid salts of basic residues such as amines; alkali or organic salts of acidic residues such as carboxylic acids; and the like. For example, such salts include salts from benzenesulfonic acid, benzoic acid, citric acid, ethane sulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. In embodiments, pharmaceutically acceptable salts are selected from chloride and fumarate salts.

Pharmaceutically acceptable salts can be synthesized from zongertinib by conventional chemical methods. Generally, such salts can be prepared by reacting the free base form of zongertinib with a sufficient amount of the appropriate acid or base in water or in an organic diluent or solvent like ether, ethyl acetate, ethanol, isopropanol, or acetonitrile, or a mixture thereof.

The term “solvate” as used herein refers to an association or complex of one or more solvent molecules and zongertinib. Examples of solvents include water, isopropanol, ethanol, methanol, dimethyl sulfoxide (DMSO), ethyl acetate, acetic acid, tert-butyl methyl ether, tetrahydrofuran, methylethyl ketone, N-methylpyrrolidone and ethanolamine. The term “hydrate” refers to a complex where the solvent molecule is water.

In embodiments, zongertinib is in an amorphous form. In embodiments, zongertinib is in a crystalline form, for example in one of the crystalline forms described in WO 2024/133302, which is herein incorporated by reference in its entirety.

### KRAS G12C Inhibitors

The term “KRAS G12C inhibitor” as used herein refers to a compound that inhibits and/or reduces the biological activity of KRAS exhibiting a G12C mutation. KRAS G12C inhibitors belonging to different compound classes are known. The term “KRAS G12C inhibitor” as used herein also encompasses any tautomers, pharmaceutically acceptable salts, solid state forms of any KRAS G12C inhibitor, as well as solvates, including hydrates and solvates of pharmaceutically acceptable salts thereof.

Examples of KRAS G12C inhibitors are sotorasib and adagrasib. They are KRAS G12C selective inhibitors for which clinical data have been reported. Adagrasib is also known under lab code MRTX 849 and commercial name Lumakras. For example, WO 2017/201161 and WO 2019/099524 describe general reaction schemes for its preparation, synthetic routes, and the properties. Sotorasib is also known under lab code AMG 510 and commercial name Krazati. For example, WO 2018/217651 and WO 2020/102730 describe general reaction schemes for its preparation, synthetic routes, and the properties.

Further examples of KRAS G12C inhibitors are disclosed in WO2021/245051, WO2021/245055 and WO2023/099612, which are herein incorporated by reference in their entireties. In particular, further examples of KRAS G12C inhibitors that can be used according to the present invention are represented by:
compounds Ib-1 to Ib-16, Ic-1 to Ic-9, Id-1 to Id-9 and Ie-1 of WO2021/245051:

| # | structure |
|---|---|
| Ib-1 | 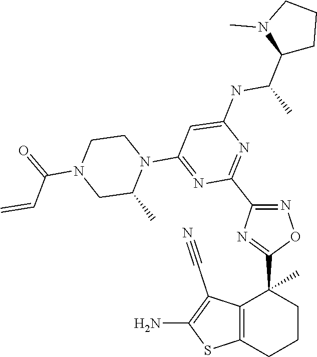 [SMILES: C=CC(=O)N1CCN(c2cc(N[C@@H](C)[C@@H]3CCCN3C)nc(-c3noc([C@@]4(C)CCCc5sc(N)c(C#N)c54)n3)n2)[C@H](C)C1] |
| Ib-2 | 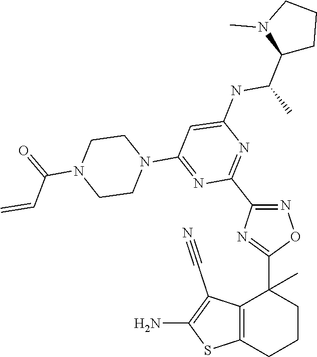 [SMILES: C=CC(=O)N1CCN(c2cc(N[C@@H](C)[C@@H]3CCCN3C)nc(-c3noc(C4(C)CCCc5sc(N)c(C#N)c54)n3)n2)CC1] |
| Ib-3 | 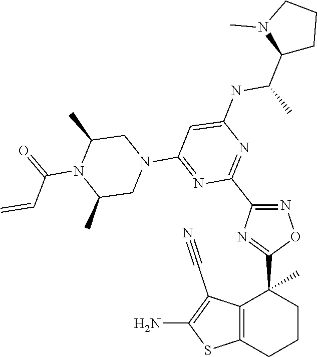 [SMILES: C=CC(=O)N1[C@H](C)CN(c2cc(N[C@@H](C)[C@@H]3CCCN3C)nc(-c3noc([C@@]4(C)CCCc5sc(N)c(C#N)c54)n3)n2)C[C@@H]1C] |
| Ib-4 | 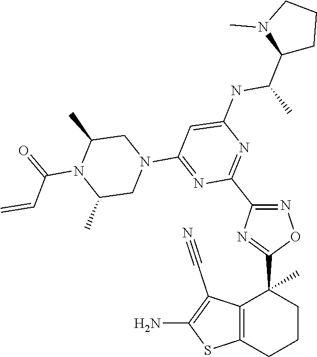 [SMILES: C=CC(=O)N1[C@@H](C)CN(c2cc(N[C@@H](C)[C@@H]3CCCN3C)nc(-c3noc([C@@]4(C)CCCc5sc(N)c(C#N)c54)n3)n2)C[C@@H]1C] |
| Ib-5 | 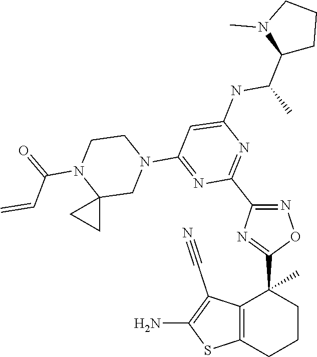 [SMILES: C=CC(=O)N1CCN(c2cc(N[C@@H](C)[C@@H]3CCCN3C)nc(-c3noc([C@@]4(C)CCCc5sc(N)c(C#N)c54)n3)n2)CC12CC2] |
| Ib-6 | 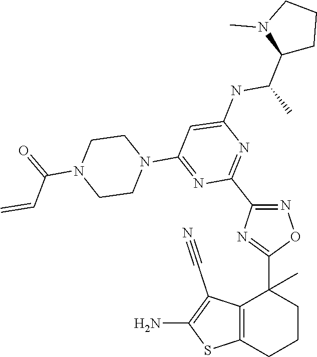 [SMILES: C=CC(=O)N1CCN(c2cc(N[C@@H](C)[C@@H]3CCCN3C)nc(-c3noc(C4(C)CCCc5sc(N)c(C#N)c54)n3)n2)CC1] |
| Ib-7 | 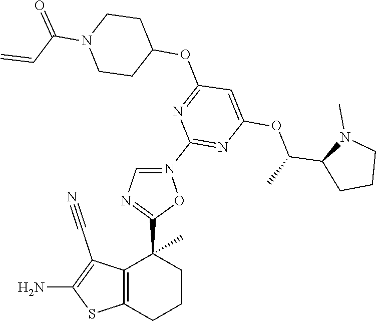 |
| Ib-8 | 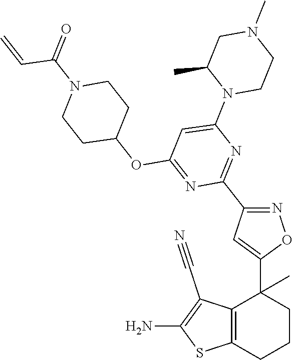 [SMILES: C=CC(=O)N1CCC(Oc2cc(N3CCN(C)C[C@@H]3C)nc(-c3cc(C4(C)CCCc5sc(N)c(C#N)c54)on3)n2)CC1] |
| Ib-9 | 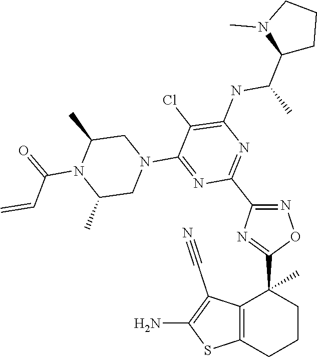 [SMILES: C=CC(=O)N1[C@@H](C)CN(c2nc(-c3noc([C@@]4(C)CCCc5sc(N)c(C#N)c54)n3)nc(N[C@@H](C)[C@@H]3CCCN3C)c2Cl)C[C@@H]1C] |
| Ic-1 | 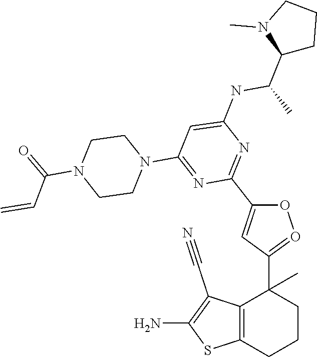 |
| Ic-2 | 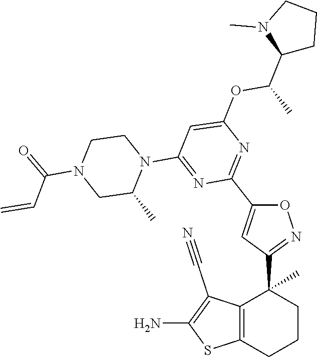 [SMILES: C=CC(=O)N1CCN(c2cc(O[C@@H](C)[C@@H]3CCCN3C)nc(-c3cc([C@@]4(C)CCCc5sc(N)c(C#N)c54)no3)n2)[C@H](C)C1] |
| Ic-3 | 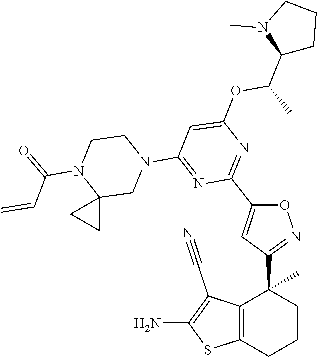 [SMILES: C=CC(=O)N1CCN(c2cc(O[C@@H](C)[C@@H]3CCCN3C)nc(-c3cc([C@@]4(C)CCCc5sc(N)c(C#N)c54)no3)n2)CC12CC2] |
| Ic-4 | 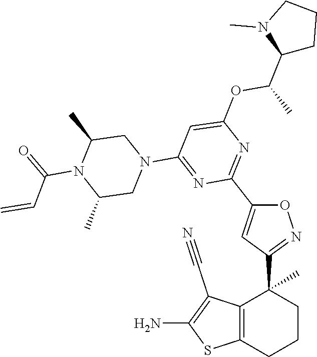 [SMILES: C=CC(=O)N1[C@@H](C)CN(c2cc(O[C@@H](C)[C@@H]3CCCN3C)nc(-c3cc([C@@]4(C)CCCc5sc(N)c(C#N)c54)no3)n2)C[C@@H]1C] |
| Ic-5 | 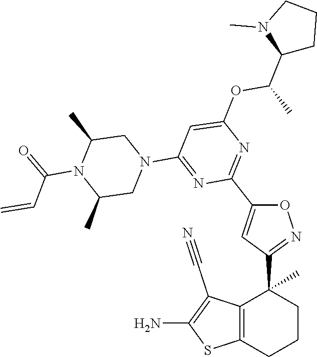 [SMILES: C=CC(=O)N1[C@H](C)CN(c2cc(O[C@@H](C)[C@@H]3CCCN3C)nc(-c3cc([C@@]4(C)CCCc5sc(N)c(C#N)c54)no3)n2)C[C@@H]1C] |
| Ic-6 | 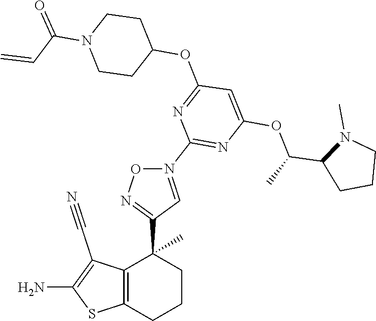 |
| Ic-7 | 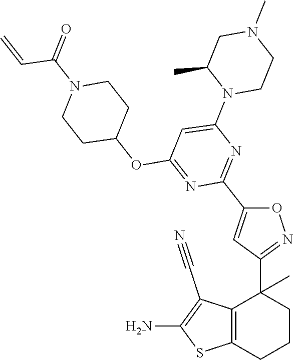 [SMILES: C=CC(=O)N1CCC(Oc2cc(N3CCN(C)C[C@@H]3C)nc(-c3cc(C4(C)CCCc5sc(N)c(C#N)c54)no3)n2)CC1] |
| Id-1 | 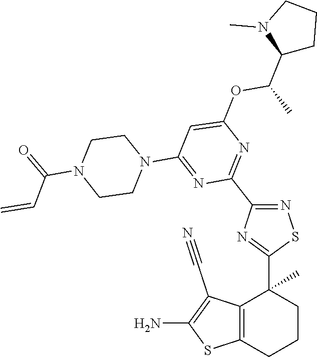 [SMILES: C=CC(=O)N1CCN(c2cc(O[C@@H](C)[C@@H]3CCCN3C)nc(-c3nsc([C@@]4(C)CCCc5sc(N)c(C#N)c54)n3)n2)CC1] |
| Id-2 | 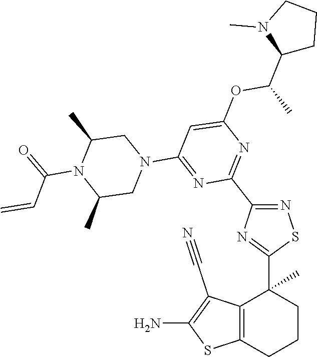 [SMILES: C=CC(=O)N1[C@H](C)CN(c2cc(O[C@@H](C)[C@@H]3CCCN3C)nc(-c3nsc([C@@]4(C)CCCc5sc(N)c(C#N)c54)n3)n2)C[C@@H]1C] |
| Id-3 | 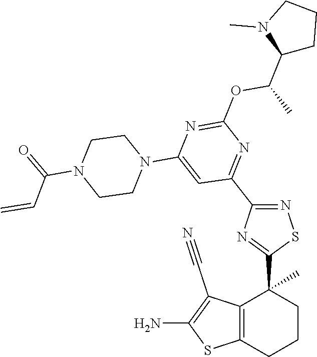 [SMILES: C=CC(=O)N1CCN(c2cc(-c3nsc([C@@]4(C)CCCc5sc(N)c(C#N)c54)n3)nc(O[C@@H](C)[C@@H]3CCCN3C)n2)CC1] |
| Id-4 | 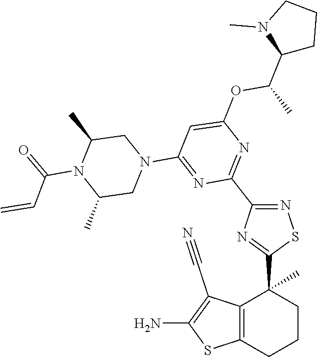 [SMILES: C=CC(=O)N1[C@@H](C)CN(c2cc(O[C@@H](C)[C@@H]3CCCN3C)nc(-c3nsc([C@@]4(C)CCCc5sc(N)c(C#N)c54)n3)n2)C[C@@H]1C] |
| Id-5 | 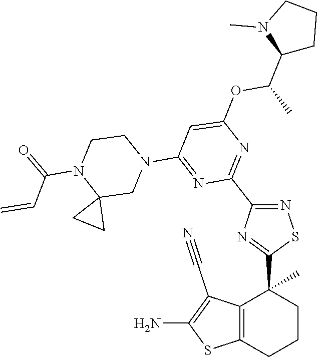 [SMILES: C=CC(=O)N1CCN(c2cc(O[C@@H](C)[C@@H]3CCCN3C)nc(-c3nsc([C@@]4(C)CCCc5sc(N)c(C#N)c54)n3)n2)CC12CC2] |
| Id-6 | 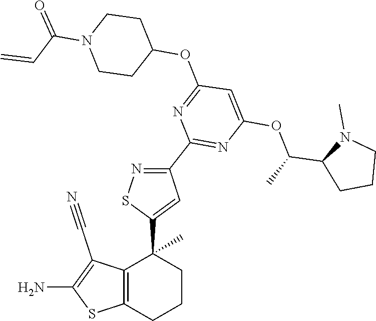 [SMILES: C=CC(=O)N1CCC(Oc2cc(O[C@@H](C)[C@@H]3CCCN3C)nc(-c3cc([C@@]4(C)CCCc5sc(N)c(C#N)c54)sn3)n2)CC1] |
| Id-7 | 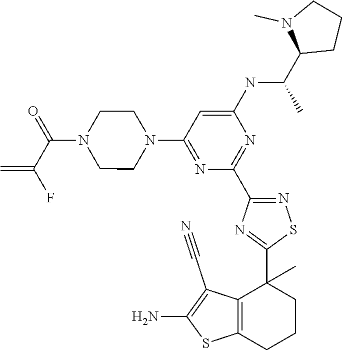 [SMILES: C=C(F)C(=O)N1CCN(c2cc(N[C@@H](C)[C@@H]3CCCN3C)nc(-c3nsc(C4(C)CCCc5sc(N)c(C#N)c54)n3)n2)CC1] |
| Id-8 | 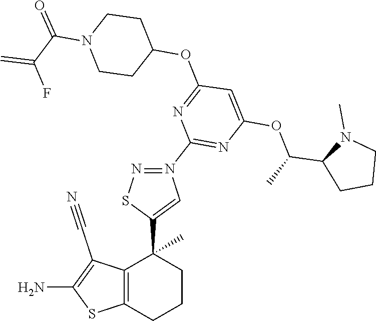 |
| Ib-10 | 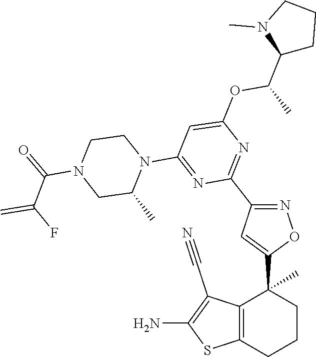 [SMILES: C=C(F)C(=O)N1CCN(c2cc(O[C@@H](C)[C@@H]3CCCN3C)nc(-c3cc([C@@]4(C)CCCc5sc(N)c(C#N)c54)on3)n2)[C@H](C)C1] |
| Ib-11 | 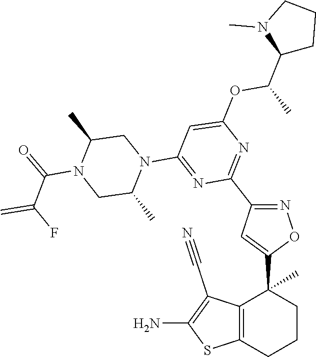 [SMILES: C=C(F)C(=O)N1C[C@@H](C)N(c2cc(O[C@@H](C)[C@@H]3CCCN3C)nc(-c3cc([C@@]4(C)CCCc5sc(N)c(C#N)c54)on3)n2)C[C@@H]1C] |
| Ib-12 | 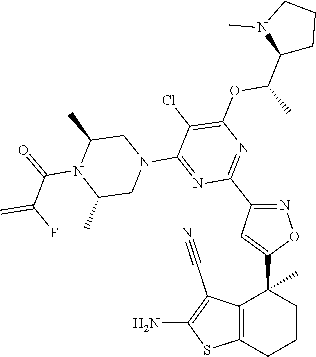 [SMILES: C=C(F)C(=O)N1[C@@H](C)CN(c2nc(-c3cc([C@@]4(C)CCCc5sc(N)c(C#N)c54)on3)nc(O[C@@H](C)[C@@H]3CCCN3C)c2Cl)C[C@@H]1C] |
| Ib-13 | 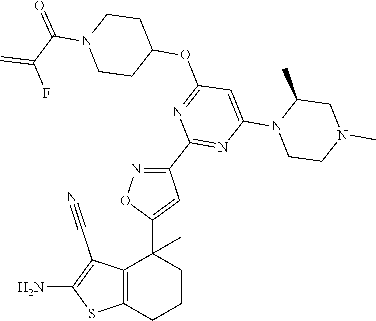 [SMILES: C=C(F)C(=O)N1CCC(Oc2cc(N3CCN(C)C[C@@H]3C)nc(-c3cc(C4(C)CCCc5sc(N)c(C#N)c54)on3)n2)CC1] |
| Ic-8 | 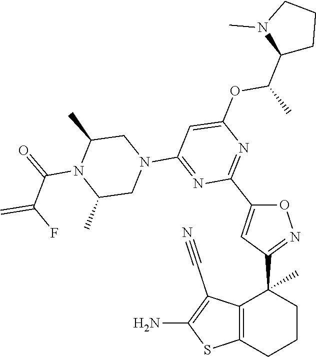 [SMILES: C=C(F)C(=O)N1[C@@H](C)CN(c2cc(O[C@@H](C)[C@@H]3CCCN3C)nc(-c3cc([C@@]4(C)CCCc5sc(N)c(C#N)c54)no3)n2)C[C@@H]1C] |
| Ib-14 | 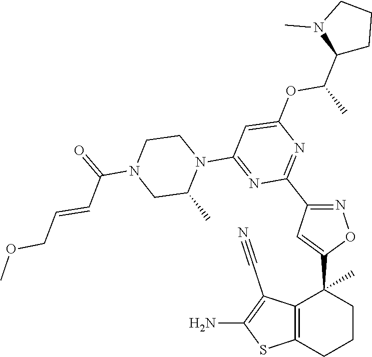 [SMILES: COC/C=C/C(=O)N1CCN(c2cc(O[C@@H](C)[C@@H]3CCCN3C)nc(-c3cc([C@@]4(C)CCCc5sc(N)c(C#N)c54)on3)n2)[C@H](C)C1] |
| Ib-15 | 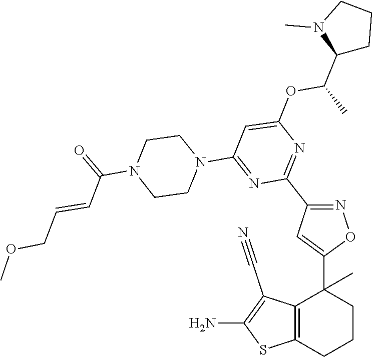 [SMILES: COC/C=C/C(=O)N1CCN(c2cc(O[C@@H](C)[C@@H]3CCCN3C)nc(-c3cc(C4(C)CCCc5sc(N)c(C#N)c54)on3)n2)CC1] |
| Ib-16 | 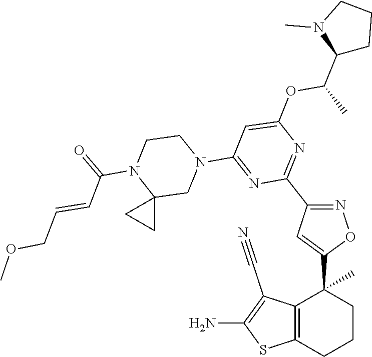 [SMILES: COC/C=C/C(=O)N1CCN(c2cc(O[C@@H](C)[C@@H]3CCCN3C)nc(-c3cc([C@@]4(C)CCCc5sc(N)c(C#N)c54)on3)n2)CC12CC2] |
| Ic-9 | 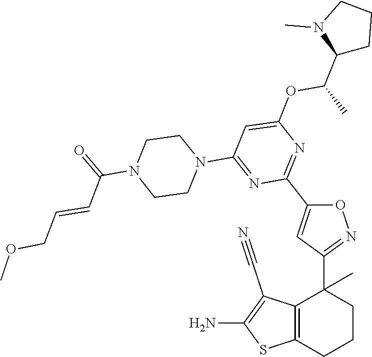 [SMILES: COC/C=C/C(=O)N1CCN(c2cc(O[C@@H](C)[C@@H]3CCCN3C)nc(-c3cc(C4(C)CCCc5sc(N)c(C#N)c54)no3)n2)CC1] |
| Id-9 | 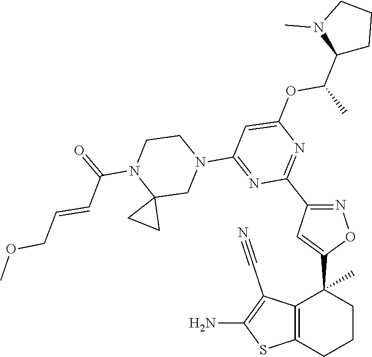 [SMILES: COC/C=C/C(=O)N1CCN(c2cc(O[C@@H](C)[C@@H]3CCCN3C)nc(-c3cc([C@@]4(C)CCCc5sc(N)c(C#N)c54)on3)n2)CC12CC2] |
| Ie-1 | 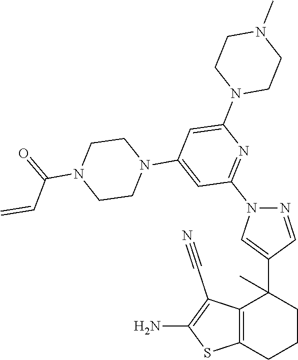 [SMILES: C=CC(=O)N1CCN(c2cc(N3CCN(C)CC3)nc(-n3cc(C4(C)CCCc5sc(N)c(C#N)c54)cn3)c2)CC1] |

compounds Ia-1 to Ia-170 of WO2021/245055:

| # | structure |
|---|---|
| Ia-1 | 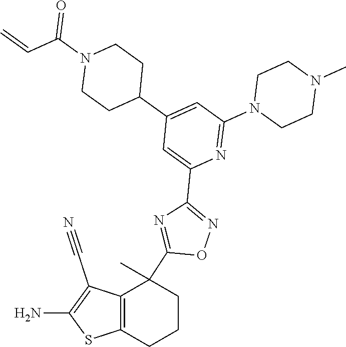 [SMILES: C=CC(=O)N1CCC(c2cc(-c3noc(C4(C)CCCc5sc(N)c(C#N)c54)n3)nc(N3CCN(C)CC3)c2)CC1] |
| Ia-2 | 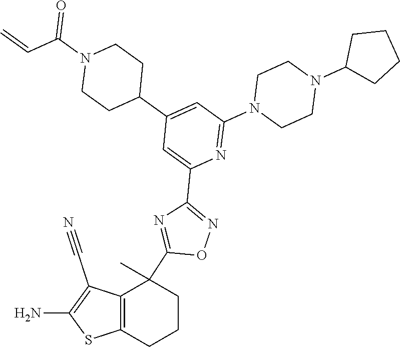 [SMILES: C=CC(=O)N1CCC(c2cc(-c3noc(C4(C)CCCc5sc(N)c(C#N)c54)n3)nc(N3CCN(C4CCCC4)CC3)c2)CC1] |
| Ia-3 | 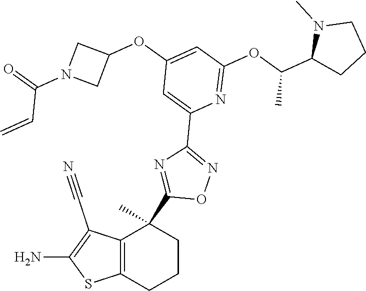 [SMILES: C=CC(=O)N1CC(Oc2cc(O[C@@H](C)[C@@H]3CCCN3C)nc(-c3noc([C@@]4(C)CCCc5sc(N)c(C#N)c54)n3)c2)C1] |
| Ia-4 | 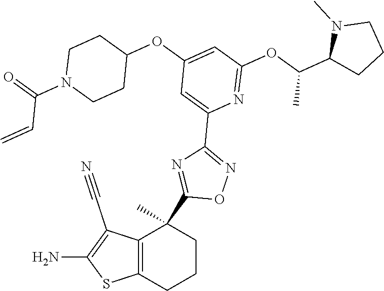 [SMILES: C=CC(=O)N1CCC(Oc2cc(O[C@@H](C)[C@@H]3CCCN3C)nc(-c3noc([C@@]4(C)CCCc5sc(N)c(C#N)c54)n3)c2)CC1] |
| Ia-5 | 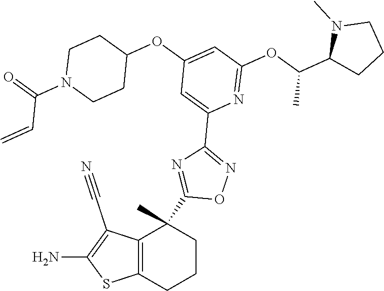 [SMILES: C=CC(=O)N1CCC(Oc2cc(O[C@@H](C)[C@@H]3CCCN3C)nc(-c3noc([C@]4(C)CCCc5sc(N)c(C#N)c54)n3)c2)CC1] |
| Ia-6 | 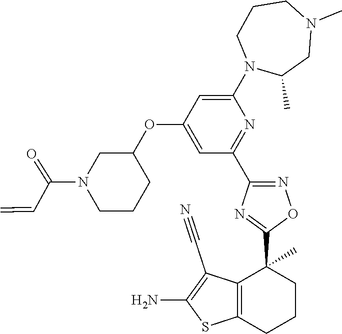 [SMILES: C=CC(=O)N1CCCC(Oc2cc(-c3noc([C@@]4(C)CCCc5sc(N)c(C#N)c54)n3)nc(N3CCCN(C)C[C@@H]3C)c2)C1] |
| Ia-7 | 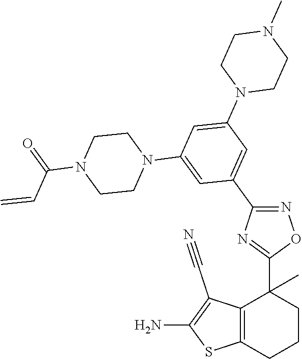 [SMILES: C=CC(=O)N1CCN(c2cc(-c3noc(C4(C)CCCc5sc(N)c(C#N)c54)n3)cc(N3CCN(C)CC3)c2)CC1] |
| Ia-8 | 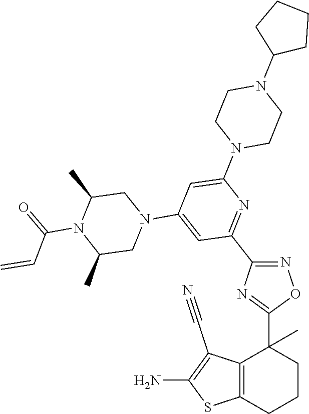 [SMILES: C=CC(=O)N1[C@H](C)CN(c2cc(-c3noc(C4(C)CCCc5sc(N)c(C#N)c54)n3)nc(N3CCN(C4CCCC4)CC3)c2)C[C@@H]1C] |
| Ia-9 | 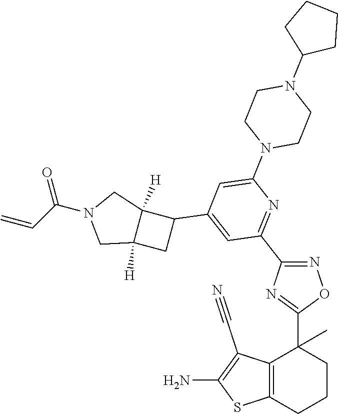 [SMILES: C=CC(=O)N1C[C@@H]2CC(c3cc(-c4noc(C5(C)CCCc6sc(N)c(C#N)c65)n4)nc(N4CCN(C5CCCC5)CC4)c3)[C@@H]2C1] |
| Ia-10 | 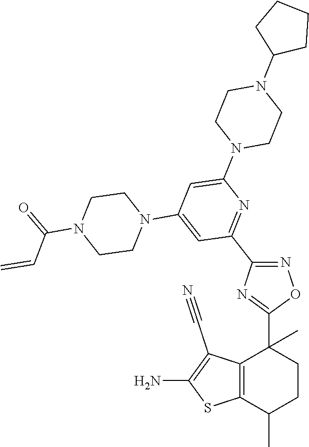 [SMILES: C=CC(=O)N1CCN(c2cc(-c3noc(C4(C)CCC(C)c5sc(N)c(C#N)c54)n3)nc(N3CCN(C4CCCC4)CC3)c2)CC1] |
| Ia-11 | 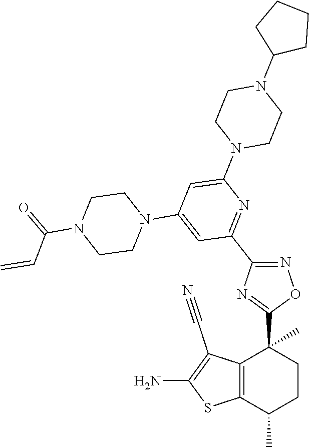 [SMILES: C=CC(=O)N1CCN(c2cc(-c3noc([C@@]4(C)CC[C@H](C)c5sc(N)c(C#N)c54)n3)nc(N3CCN(C4CCCC4)CC3)c2)CC1] |
| Ia-12 | 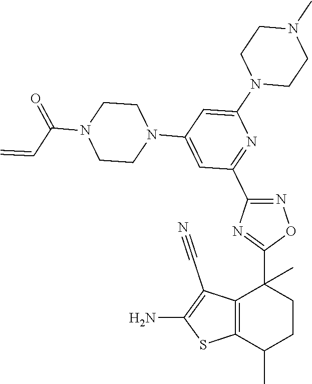 [SMILES: C=CC(=O)N1CCN(c2cc(-c3noc(C4(C)CCC(C)c5sc(N)c(C#N)c54)n3)nc(N3CCN(C)CC3)c2)CC1] |
| Ia-13 | 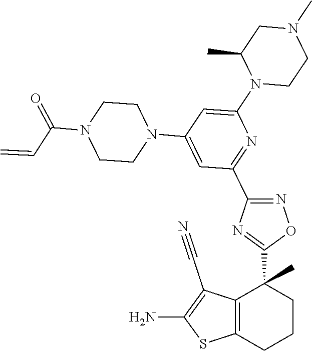 [SMILES: C=CC(=O)N1CCN(c2cc(-c3noc([C@]4(C)CCCc5sc(N)c(C#N)c54)n3)nc(N3CCN(C)C[C@@H]3C)c2)CC1] |
| Ia-14 | 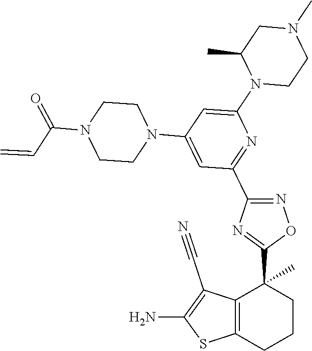 [SMILES: C=CC(=O)N1CCN(c2cc(-c3noc([C@@]4(C)CCCc5sc(N)c(C#N)c54)n3)nc(N3CCN(C)C[C@@H]3C)c2)CC1] |
| Ia-15 | 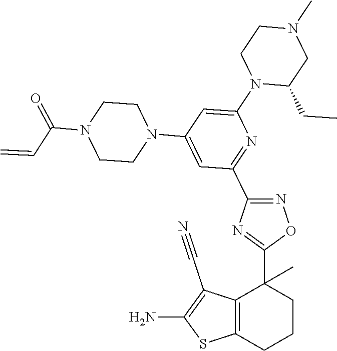 [SMILES: C=CC(=O)N1CCN(c2cc(-c3noc(C4(C)CCCc5sc(N)c(C#N)c54)n3)nc(N3CCN(C)C[C@@H]3CC)c2)CC1] |
| Ia-16 | 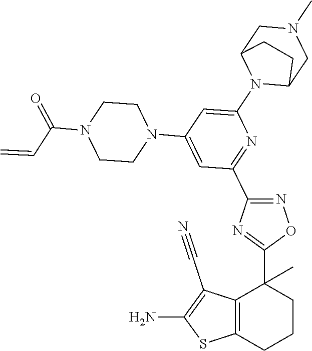 [SMILES: C=CC(=O)N1CCN(c2cc(-c3noc(C4(C)CCCc5sc(N)c(C#N)c54)n3)nc(N3C4CCC3CN(C)C4)c2)CC1] |
| Ia-17 | 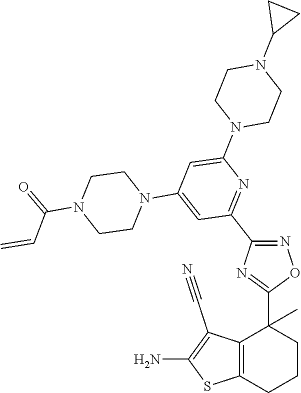 [SMILES: C=CC(=O)N1CCN(c2cc(-c3noc(C4(C)CCCc5sc(N)c(C#N)c54)n3)nc(N3CCN(C4CC4)CC3)c2)CC1] |
| Ia-18 | 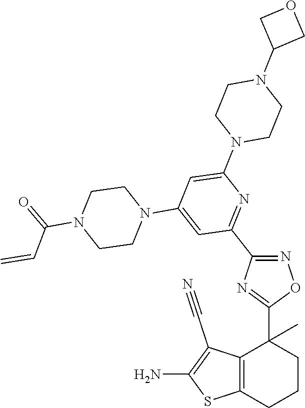 [SMILES: C=CC(=O)N1CCN(c2cc(-c3noc(C4(C)CCCc5sc(N)c(C#N)c54)n3)nc(N3CCN(C4COC4)CC3)c2)CC1] |
| Ia-19 | 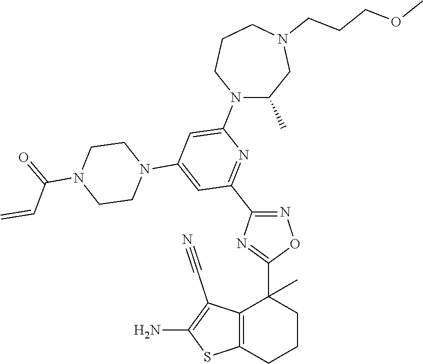 [SMILES: C=CC(=O)N1CCN(c2cc(-c3noc(C4(C)CCCc5sc(N)c(C#N)c54)n3)nc(N3CCCN(CCCOC)C[C@@H]3C)c2)CC1] |
| Ia-20 | 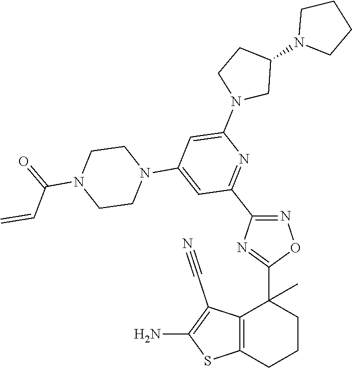 [SMILES: C=CC(=O)N1CCN(c2cc(-c3noc(C4(C)CCCc5sc(N)c(C#N)c54)n3)nc(N3CC[C@H](N4CCCC4)C3)c2)CC1] |
| Ia-21 | 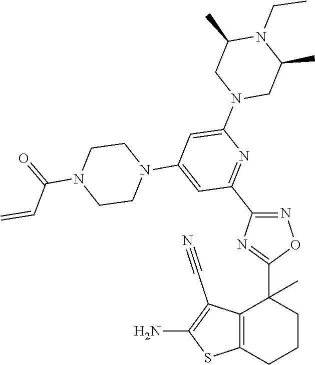 [SMILES: C=CC(=O)N1CCN(c2cc(-c3noc(C4(C)CCCc5sc(N)c(C#N)c54)n3)nc(N3C[C@@H](C)N(CC)[C@@H](C)C3)c2)CC1] |
| Ia-22 | 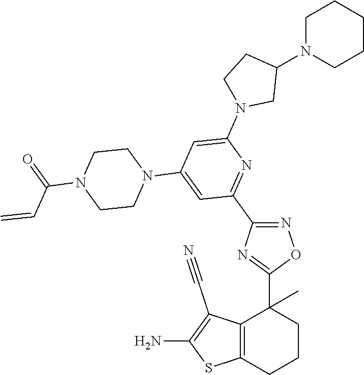 [SMILES: C=CC(=O)N1CCN(c2cc(-c3noc(C4(C)CCCc5sc(N)c(C#N)c54)n3)nc(N3CCC(N4CCCCC4)C3)c2)CC1] |
| Ia-23 | 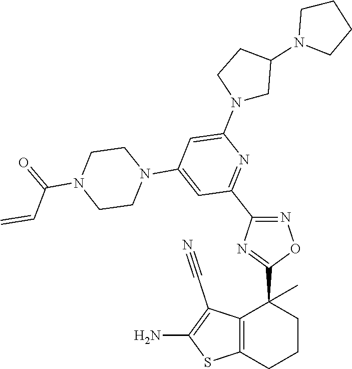 [SMILES: C=CC(=O)N1CCN(c2cc(-c3noc([C@@]4(C)CCCc5sc(N)c(C#N)c54)n3)nc(N3CCC(N4CCCC4)C3)c2)CC1] |
| Ia-24 | 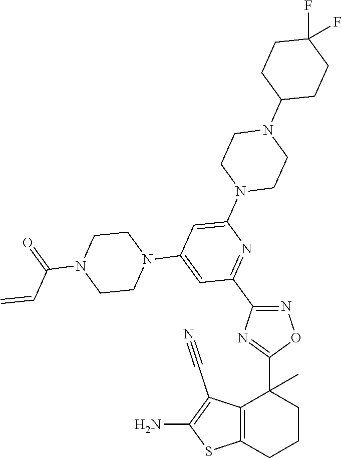 [SMILES: C=CC(=O)N1CCN(c2cc(-c3noc(C4(C)CCCc5sc(N)c(C#N)c54)n3)nc(N3CCN(C4CCC(F)(F)CC4)CC3)c2)CC1] |
| Ia-25 | 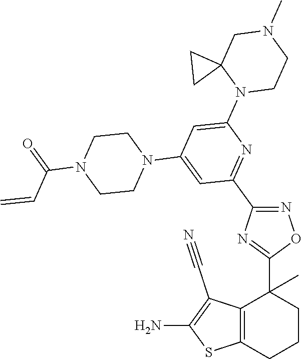 [SMILES: C=CC(=O)N1CCN(c2cc(-c3noc(C4(C)CCCc5sc(N)c(C#N)c54)n3)nc(N3CCN(C)CC34CC4)c2)CC1] |
| Ia-26 | 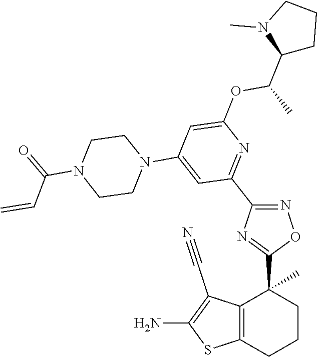 [SMILES: C=CC(=O)N1CCN(c2cc(O[C@@H](C)[C@@H]3CCCN3C)nc(-c3noc([C@@]4(C)CCCc5sc(N)c(C#N)c54)n3)c2)CC1] |
| Ia-27 | 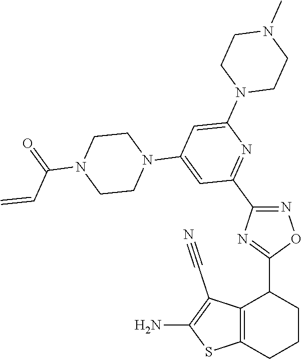 [SMILES: C=CC(=O)N1CCN(c2cc(-c3noc(C4CCCc5sc(N)c(C#N)c54)n3)nc(N3CCN(C)CC3)c2)CC1] |
| Ia-28 | 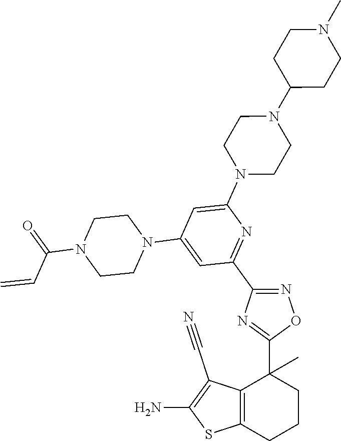 [SMILES: C=CC(=O)N1CCN(c2cc(-c3noc(C4(C)CCCc5sc(N)c(C#N)c54)n3)nc(N3CCN(C4CCN(C)CC4)CC3)c2)CC1] |
| Ia-29 | 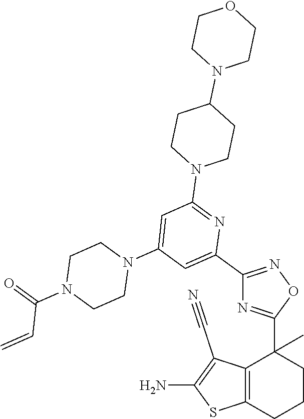 [SMILES: C=CC(=O)N1CCN(c2cc(-c3noc(C4(C)CCCc5sc(N)c(C#N)c54)n3)nc(N3CCC(N4CCOCC4)CC3)c2)CC1] |
| Ia-30 | 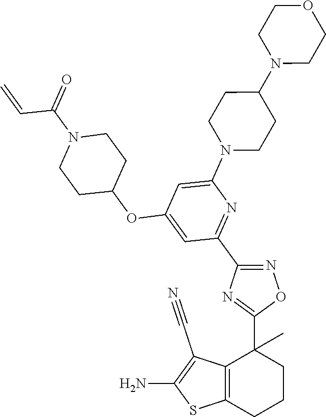 [SMILES: C=CC(=O)N1CCC(Oc2cc(-c3noc(C4(C)CCCc5sc(N)c(C#N)c54)n3)nc(N3CCC(N4CCOCC4)CC3)c2)CC1] |
| Ia-31 | 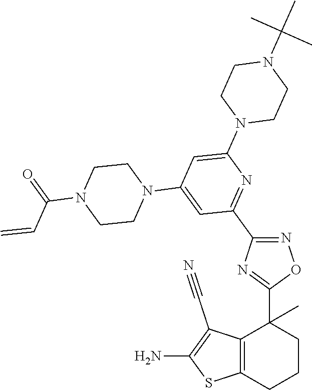 [SMILES: C=CC(=O)N1CCN(c2cc(-c3noc(C4(C)CCCc5sc(N)c(C#N)c54)n3)nc(N3CCN(C(C)(C)C)CC3)c2)CC1] |
| Ia-32 | 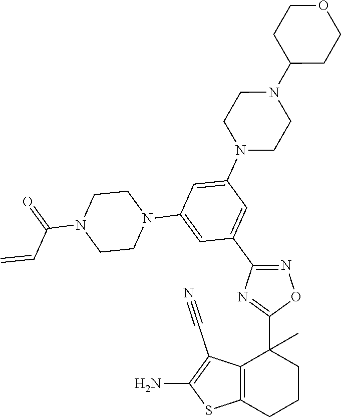 [SMILES: C=CC(=O)N1CCN(c2cc(-c3noc(C4(C)CCCc5sc(N)c(C#N)c54)n3)cc(N3CCN(C4CCOCC4)CC3)c2)CC1] |
| Ia-33 | 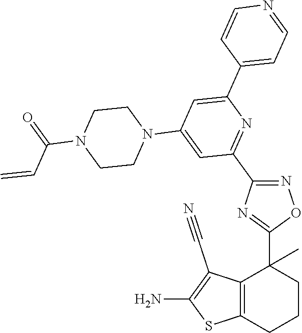 [SMILES: C=CC(=O)N1CCN(c2cc(-c3ccncc3)nc(-c3noc(C4(C)CCCc5sc(N)c(C#N)c54)n3)c2)CC1] |
| Ia-34 | 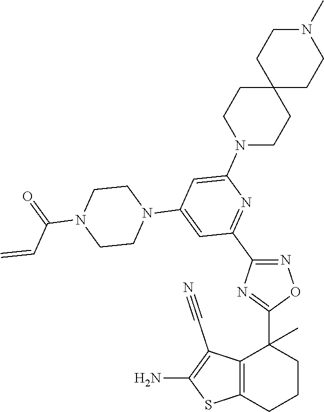 [SMILES: C=CC(=O)N1CCN(c2cc(-c3noc(C4(C)CCCc5sc(N)c(C#N)c54)n3)nc(N3CCC4(CCN(C)CC4)CC3)c2)CC1] |
| Ia-35 | 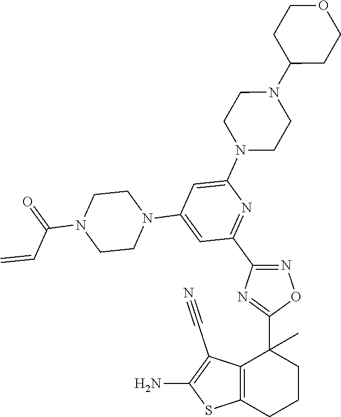 [SMILES: C=CC(=O)N1CCN(c2cc(-c3noc(C4(C)CCCc5sc(N)c(C#N)c54)n3)nc(N3CCN(C4CCOCC4)CC3)c2)CC1] |
| Ia-36 | 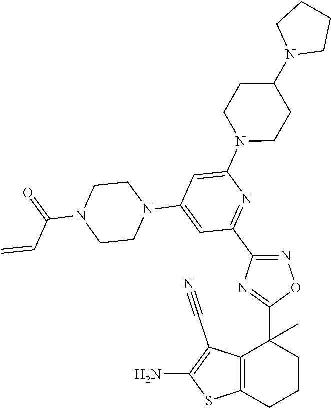 [SMILES: C=CC(=O)N1CCN(c2cc(-c3noc(C4(C)CCCc5sc(N)c(C#N)c54)n3)nc(N3CCC(N4CCCC4)CC3)c2)CC1] |
| Ia-37 | 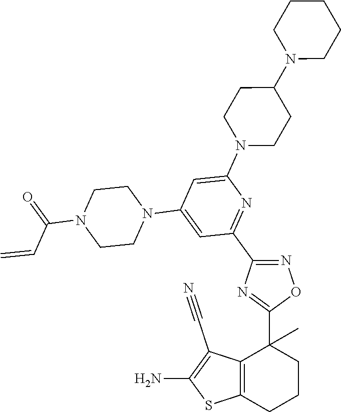 [SMILES: C=CC(=O)N1CCN(c2cc(-c3noc(C4(C)CCCc5sc(N)c(C#N)c54)n3)nc(N3CCC(N4CCCCC4)CC3)c2)CC1] |
| Ia-38 | 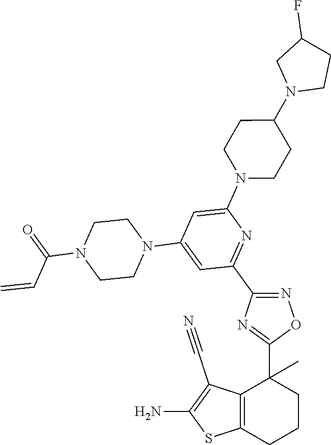 [SMILES: C=CC(=O)N1CCN(c2cc(-c3noc(C4(C)CCCc5sc(N)c(C#N)c54)n3)nc(N3CCC(N4CCC(F)C4)CC3)c2)CC1] |
| Ia-39 | 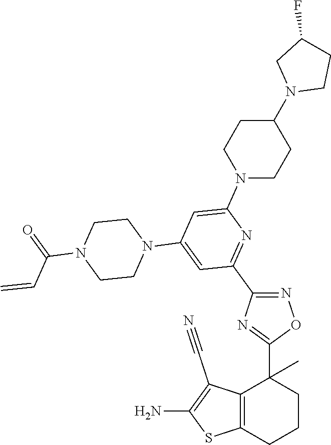 [SMILES: C=CC(=O)N1CCN(c2cc(-c3noc(C4(C)CCCc5sc(N)c(C#N)c54)n3)nc(N3CCC(N4CC[C@@H](F)C4)CC3)c2)CC1] |
| Ia-40 | 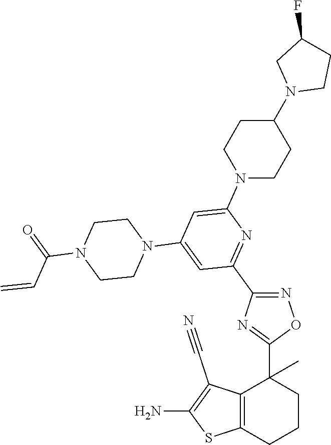 [SMILES: C=CC(=O)N1CCN(c2cc(-c3noc(C4(C)CCCc5sc(N)c(C#N)c54)n3)nc(N3CCC(N4CC[C@H](F)C4)CC3)c2)CC1] |
| Ia-41 | 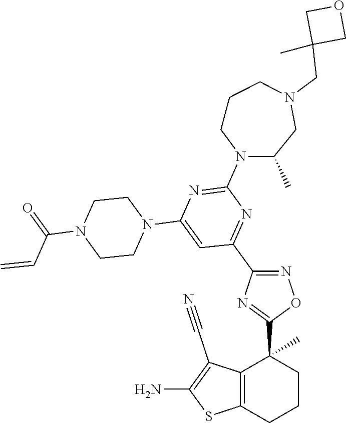 [SMILES: C=CC(=O)N1CCN(c2cc(-c3noc([C@@]4(C)CCCc5sc(N)c(C#N)c54)n3)nc(N3CCCN(CC4(C)COC4)C[C@@H]3C)n2)CC1] |
| Ia-42 | 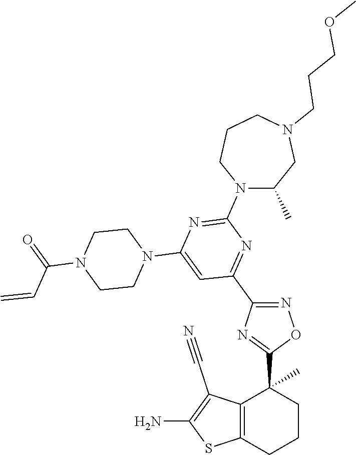 [SMILES: C=CC(=O)N1CCN(c2cc(-c3noc([C@@]4(C)CCCc5sc(N)c(C#N)c54)n3)nc(N3CCCN(CCCOC)C[C@@H]3C)n2)CC1] |
| Ia-43 | 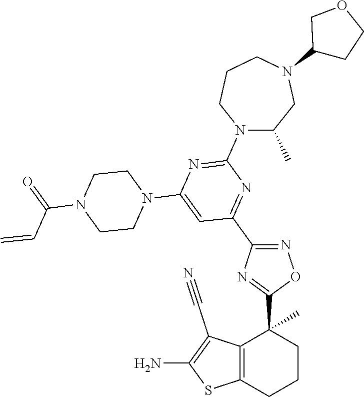 |
| Ia-44 | 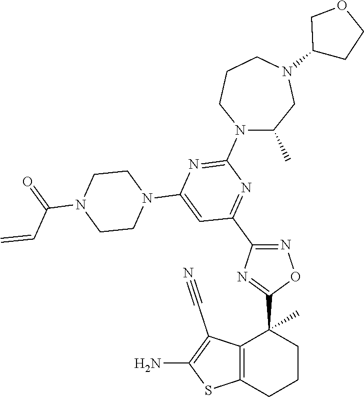 [SMILES: C=CC(=O)N1CCN(c2cc(-c3noc([C@@]4(C)CCCc5sc(N)c(C#N)c54)n3)nc(N3CCCN([C@H]4CCOC4)C[C@@H]3C)n2)CC1] |
| Ia-45 | 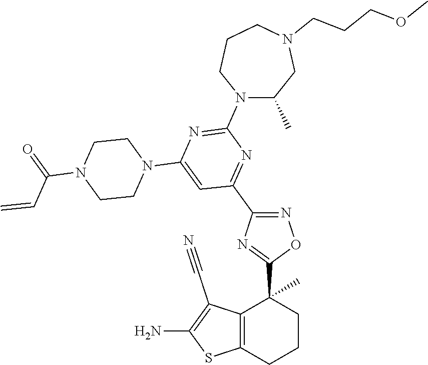 [SMILES: C=CC(=O)N1CCN(c2cc(-c3noc([C@@]4(C)CCCc5sc(N)c(C#N)c54)n3)nc(N3CCCN(CCCOC)C[C@@H]3C)n2)CC1] |
| Ia-46 | 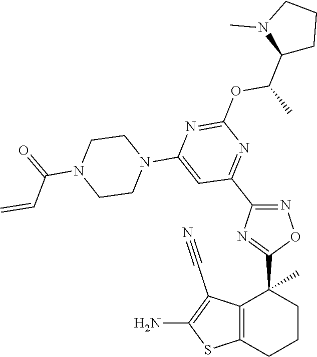 [SMILES: C=CC(=O)N1CCN(c2cc(-c3noc([C@@]4(C)CCCc5sc(N)c(C#N)c54)n3)nc(O[C@@H](C)[C@@H]3CCCN3C)n2)CC1] |
| Ia-47 | 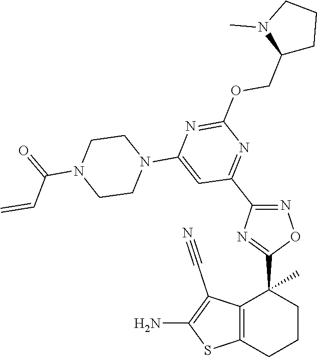 [SMILES: C=CC(=O)N1CCN(c2cc(-c3noc([C@@]4(C)CCCc5sc(N)c(C#N)c54)n3)nc(OC[C@@H]3CCCN3C)n2)CC1] |
| Ia-48 | 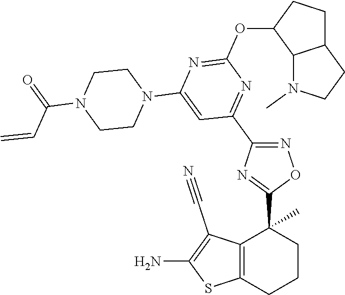 [SMILES: C=CC(=O)N1CCN(c2cc(-c3noc([C@@]4(C)CCCc5sc(N)c(C#N)c54)n3)nc(OC3CCC4CCN(C)C43)n2)CC1] |
| Ia-49 | 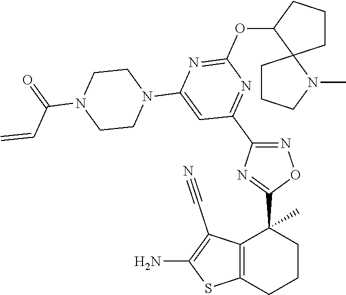 [SMILES: C=CC(=O)N1CCN(c2cc(-c3noc([C@@]4(C)CCCc5sc(N)c(C#N)c54)n3)nc(OC3CCCC34CCCN4C)n2)CC1] |
| Ia-50 | 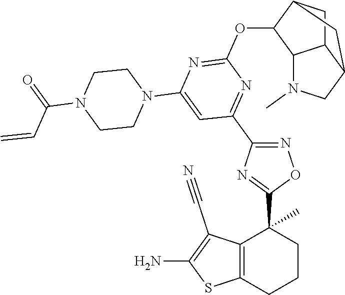 [SMILES: C=CC(=O)N1CCN(c2cc(-c3noc([C@@]4(C)CCCc5sc(N)c(C#N)c54)n3)nc(OC3C4CC5CN(C)C3C5C4)n2)CC1] |
| Ia-51 | 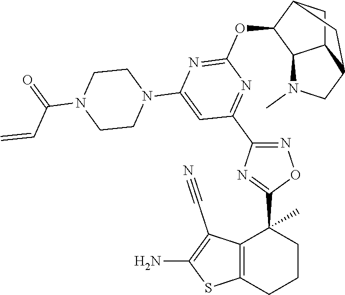 |
| Ia-52 | 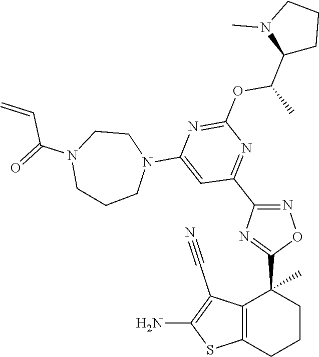 [SMILES: C=CC(=O)N1CCCN(c2cc(-c3noc([C@@]4(C)CCCc5sc(N)c(C#N)c54)n3)nc(O[C@@H](C)[C@@H]3CCCN3C)n2)CC1] |
| Ia-53 | 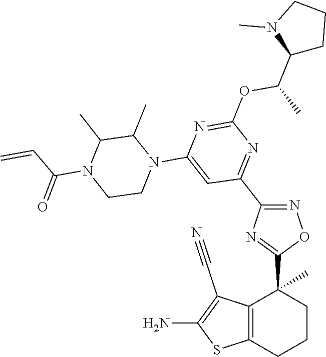 [SMILES: C=CC(=O)N1CCN(c2cc(-c3noc([C@@]4(C)CCCc5sc(N)c(C#N)c54)n3)nc(O[C@@H](C)[C@@H]3CCCN3C)n2)C(C)C1C] |
| Ia-54 | 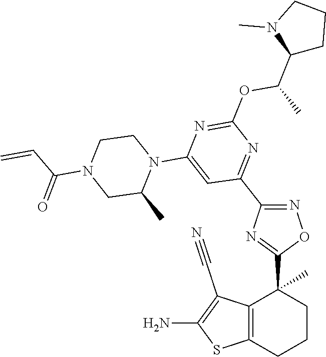 [SMILES: C=CC(=O)N1CCN(c2cc(-c3noc([C@@]4(C)CCCc5sc(N)c(C#N)c54)n3)nc(O[C@@H](C)[C@@H]3CCCN3C)n2)[C@@H](C)C1] |
| Ia-55 | 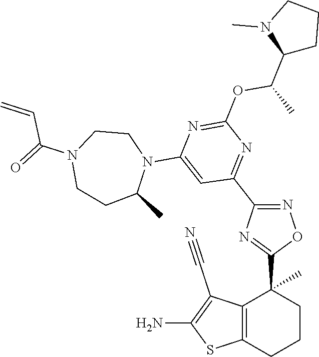 [SMILES: C=CC(=O)N1CC[C@H](C)N(c2cc(-c3noc([C@@]4(C)CCCc5sc(N)c(C#N)c54)n3)nc(O[C@@H](C)[C@@H]3CCCN3C)n2)CC1] |
| Ia-56 | 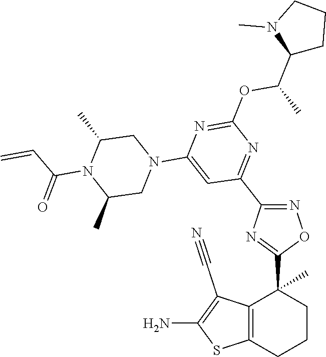 [SMILES: C=CC(=O)N1[C@H](C)CN(c2cc(-c3noc([C@@]4(C)CCCc5sc(N)c(C#N)c54)n3)nc(O[C@@H](C)[C@@H]3CCCN3C)n2)C[C@H]1C] |
| Ia-57 | 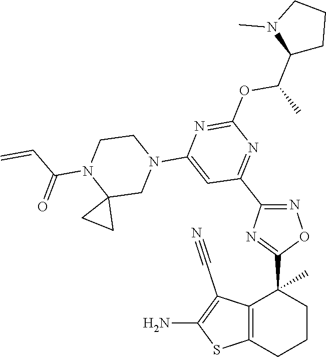 [SMILES: C=CC(=O)N1CCN(c2cc(-c3noc([C@@]4(C)CCCc5sc(N)c(C#N)c54)n3)nc(O[C@@H](C)[C@@H]3CCCN3C)n2)CC12CC2] |
| Ia-58 | 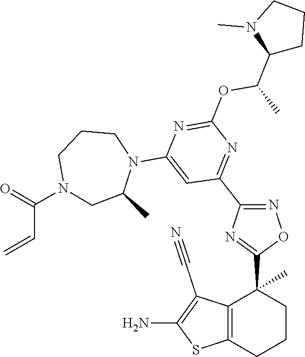 [SMILES: C=CC(=O)N1CCCN(c2cc(-c3noc([C@@]4(C)CCCc5sc(N)c(C#N)c54)n3)nc(O[C@@H](C)[C@@H]3CCCN3C)n2)[C@@H](C)C1] |
| Ia-59 | 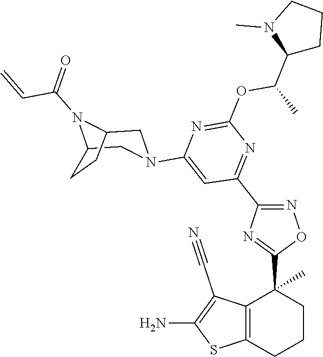 [SMILES: C=CC(=O)N1C2CCC1CN(c1cc(-c3noc([C@@]4(C)CCCc5sc(N)c(C#N)c54)n3)nc(O[C@@H](C)[C@@H]3CCCN3C)n1)C2] |
| Ia-60 | 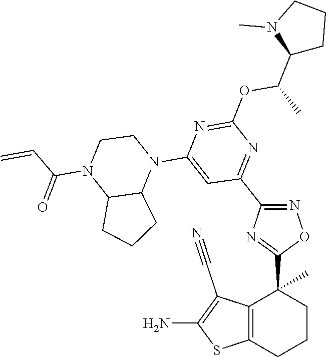 [SMILES: C=CC(=O)N1CCN(c2cc(-c3noc([C@@]4(C)CCCc5sc(N)c(C#N)c54)n3)nc(O[C@@H](C)[C@@H]3CCCN3C)n2)C2CCCC21] |
| Ia-61 | 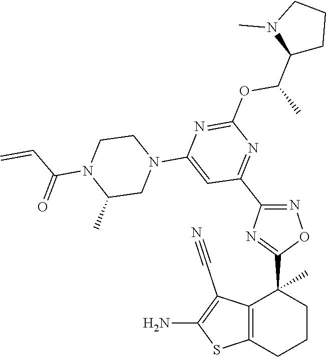 [SMILES: C=CC(=O)N1CCN(c2cc(-c3noc([C@@]4(C)CCCc5sc(N)c(C#N)c54)n3)nc(O[C@@H](C)[C@@H]3CCCN3C)n2)C[C@@H]1C] |
| Ia-62 | 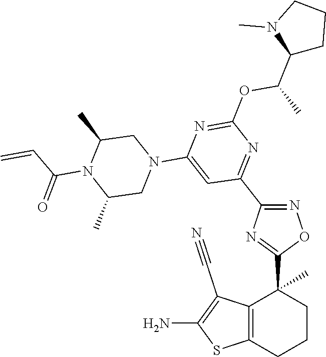 [SMILES: C=CC(=O)N1[C@@H](C)CN(c2cc(-c3noc([C@@]4(C)CCCc5sc(N)c(C#N)c54)n3)nc(O[C@@H](C)[C@@H]3CCCN3C)n2)C[C@@H]1C] |
| Ia-63 | 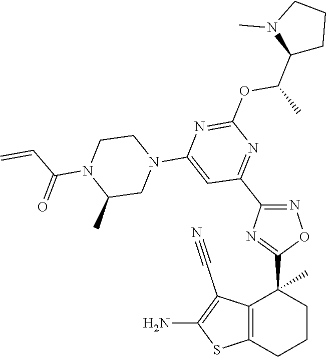 [SMILES: C=CC(=O)N1CCN(c2cc(-c3noc([C@@]4(C)CCCc5sc(N)c(C#N)c54)n3)nc(O[C@@H](C)[C@@H]3CCCN3C)n2)C[C@H]1C] |
| Ia-64 | 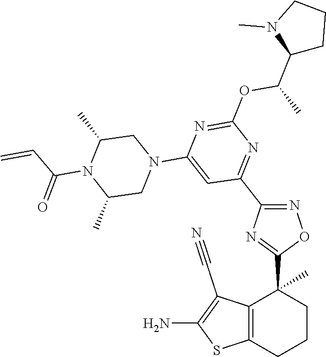 [SMILES: C=CC(=O)N1[C@H](C)CN(c2cc(-c3noc([C@@]4(C)CCCc5sc(N)c(C#N)c54)n3)nc(O[C@@H](C)[C@@H]3CCCN3C)n2)C[C@@H]1C] |
| Ia-65 | 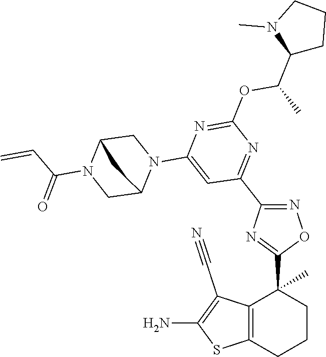 [SMILES: C=CC(=O)N1CC2C[C@H]1CN2c1cc(-c2noc([C@@]3(C)CCCc4sc(N)c(C#N)c43)n2)nc(O[C@@H](C)[C@@H]2CCCN2C)n1] |
| Ia-66 | 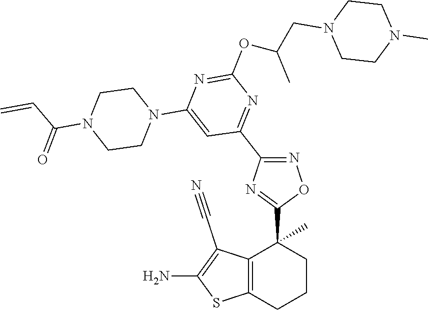 [SMILES: C=CC(=O)N1CCN(c2cc(-c3noc([C@@]4(C)CCCc5sc(N)c(C#N)c54)n3)nc(OC(C)CN3CCN(C)CC3)n2)CC1] |
| Ia-67 | 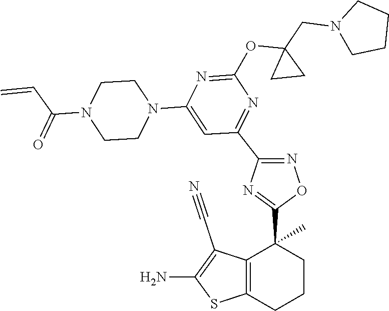 [SMILES: C=CC(=O)N1CCN(c2cc(-c3noc([C@@]4(C)CCCc5sc(N)c(C#N)c54)n3)nc(OC3(CN4CCCC4)CC3)n2)CC1] |
| Ia-68 | 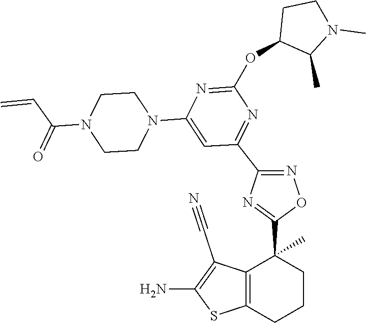 [SMILES: C=CC(=O)N1CCN(c2cc(-c3noc([C@@]4(C)CCCc5sc(N)c(C#N)c54)n3)nc(O[C@H]3CCN(C)[C@H]3C)n2)CC1] |
| Ia-69 | 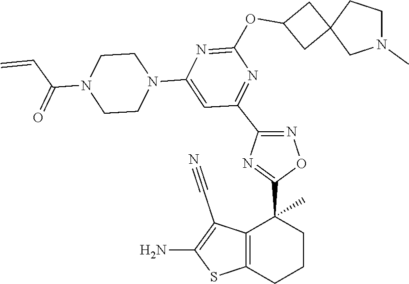 [SMILES: C=CC(=O)N1CCN(c2cc(-c3noc([C@@]4(C)CCCc5sc(N)c(C#N)c54)n3)nc(OC3CC4(CCN(C)C4)C3)n2)CC1] |
| Ia-70 | 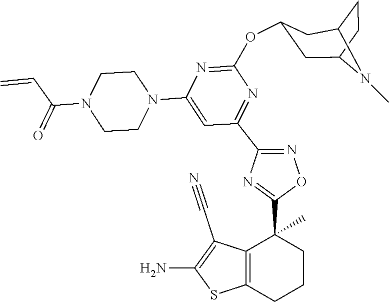 [SMILES: C=CC(=O)N1CCN(c2cc(-c3noc([C@@]4(C)CCCc5sc(N)c(C#N)c54)n3)nc(OC3CC4CCC(C3)N4C)n2)CC1] |
| Ia-71 | 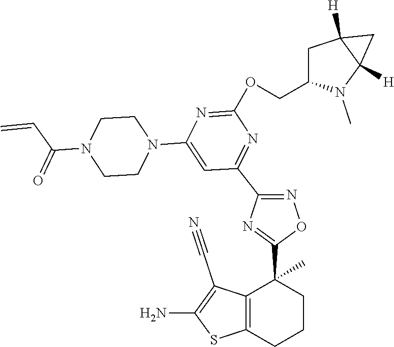 [SMILES: C=CC(=O)N1CCN(c2cc(-c3noc([C@@]4(C)CCCc5sc(N)c(C#N)c54)n3)nc(OC[C@@H]3C[C@@H]4C[C@@H]4N3C)n2)CC1] |
| Ia-72 | 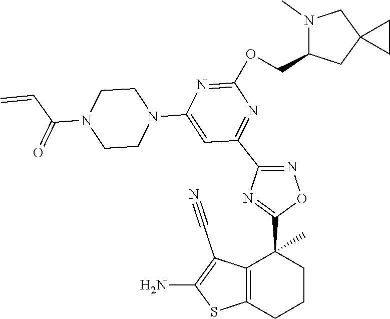 [SMILES: C=CC(=O)N1CCN(c2cc(-c3noc([C@@]4(C)CCCc5sc(N)c(C#N)c54)n3)nc(OC[C@@H]3CC4(CC4)CN3C)n2)CC1] |
| Ia-73 | 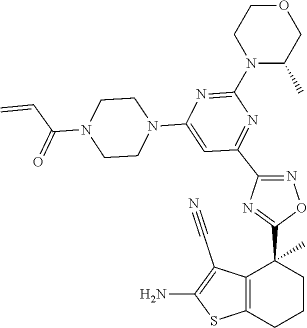 [SMILES: C=CC(=O)N1CCN(c2cc(-c3noc([C@@]4(C)CCCc5sc(N)c(C#N)c54)n3)nc(N3CCOC[C@@H]3C)n2)CC1] |
| Ia-74 | 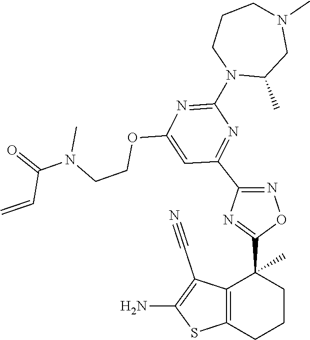 [SMILES: C=CC(=O)N(C)CCOc1cc(-c2noc([C@@]3(C)CCCc4sc(N)c(C#N)c43)n2)nc(N2CCCN(C)C[C@@H]2C)n1] |
| Ia-75 | 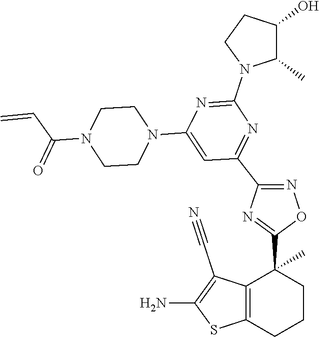 [SMILES: C=CC(=O)N1CCN(c2cc(-c3noc([C@@]4(C)CCCc5sc(N)c(C#N)c54)n3)nc(N3CC[C@H](O)[C@@H]3C)n2)CC1] |
| Ia-76 | 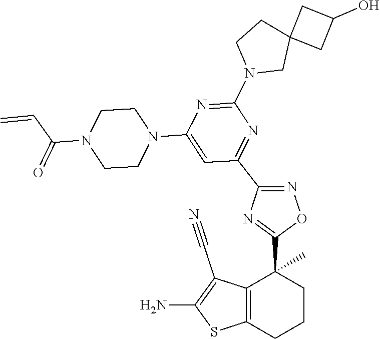 [SMILES: C=CC(=O)N1CCN(c2cc(-c3noc([C@@]4(C)CCCc5sc(N)c(C#N)c54)n3)nc(N3CCC4(CC(O)C4)C3)n2)CC1] |
| Ia-77 | 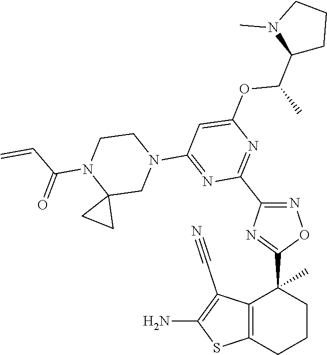 [SMILES: C=CC(=O)N1CCN(c2cc(O[C@@H](C)[C@@H]3CCCN3C)nc(-c3noc([C@@]4(C)CCCc5sc(N)c(C#N)c54)n3)n2)CC12CC2] |
| Ia-78 | 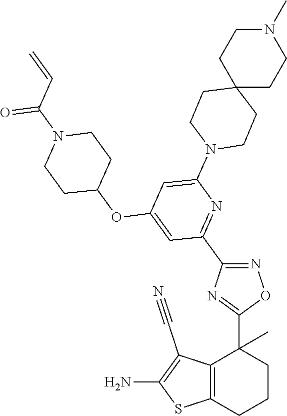 [SMILES: C=CC(=O)N1CCC(Oc2cc(-c3noc(C4(C)CCCc5sc(N)c(C#N)c54)n3)nc(N3CCC4(CCN(C)CC4)CC3)c2)CC1] |
| Ia-79 | 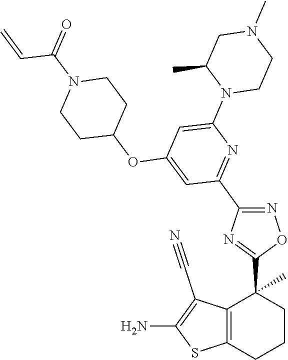 [SMILES: C=CC(=O)N1CCC(Oc2cc(-c3noc([C@@]4(C)CCCc5sc(N)c(C#N)c54)n3)nc(N3CCN(C)C[C@@H]3C)c2)CC1] |
| Ia-80 | 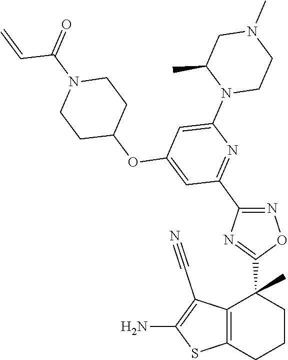 [SMILES: C=CC(=O)N1CCC(Oc2cc(-c3noc([C@]4(C)CCCc5sc(N)c(C#N)c54)n3)nc(N3CCN(C)C[C@@H]3C)c2)CC1] |
| Ia-81 | 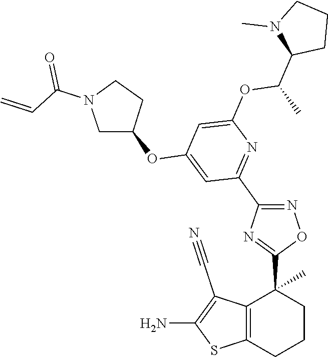 [SMILES: C=CC(=O)N1CC[C@@H](Oc2cc(O[C@@H](C)[C@@H]3CCCN3C)nc(-c3noc([C@@]4(C)CCCc5sc(N)c(C#N)c54)n3)c2)C1] |
| Ia-82 | 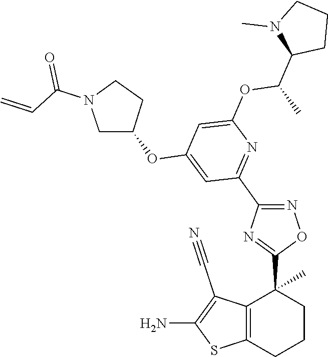 [SMILES: C=CC(=O)N1CC[C@H](Oc2cc(O[C@@H](C)[C@@H]3CCCN3C)nc(-c3noc([C@@]4(C)CCCc5sc(N)c(C#N)c54)n3)c2)C1] |
| Ia-83 | 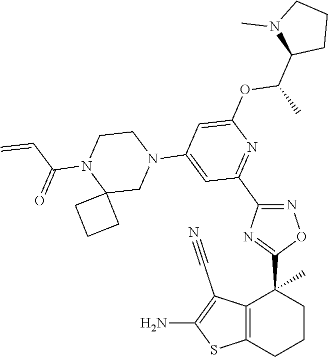 [SMILES: C=CC(=O)N1CCN(c2cc(O[C@@H](C)[C@@H]3CCCN3C)nc(-c3noc([C@@]4(C)CCCc5sc(N)c(C#N)c54)n3)c2)CC12CCC2] |
| Ia-84 | 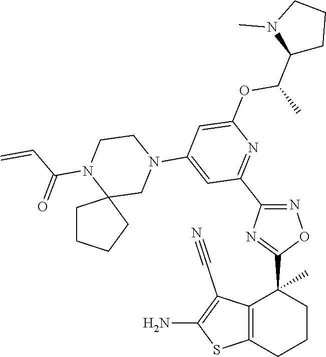 [SMILES: C=CC(=O)N1CCN(c2cc(O[C@@H](C)[C@@H]3CCCN3C)nc(-c3noc([C@@]4(C)CCCc5sc(N)c(C#N)c54)n3)c2)CC12CCCC2] |
| Ia-85 | 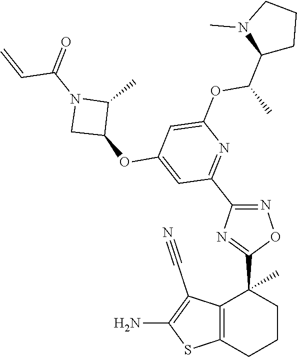 [SMILES: C=CC(=O)N1C[C@H](Oc2cc(O[C@@H](C)[C@@H]3CCCN3C)nc(-c3noc([C@@]4(C)CCCc5sc(N)c(C#N)c54)n3)c2)[C@H]1C] |
| Ia-86 | 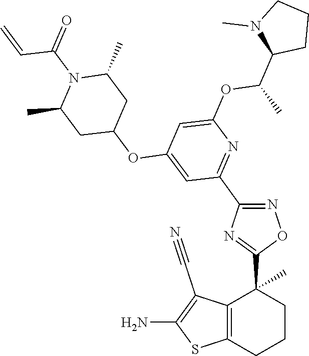 [SMILES: C=CC(=O)N1[C@H](C)CC(Oc2cc(O[C@@H](C)[C@@H]3CCCN3C)nc(-c3noc([C@@]4(C)CCCc5sc(N)c(C#N)c54)n3)c2)C[C@H]1C] |
| Ia-87 | 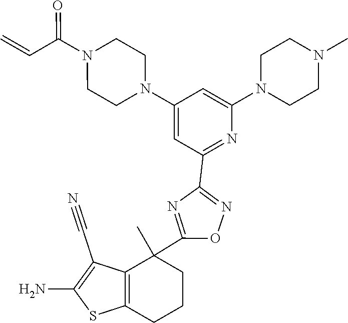 [SMILES: C=CC(=O)N1CCN(c2cc(-c3noc(C4(C)CCCc5sc(N)c(C#N)c54)n3)nc(N3CCN(C)CC3)c2)CC1] |
| Ia-88 | 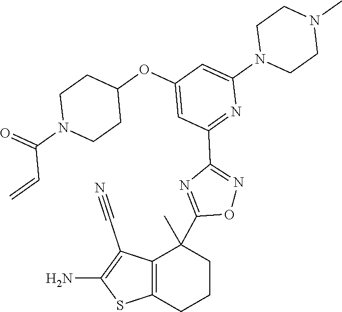 [SMILES: C=CC(=O)N1CCC(Oc2cc(-c3noc(C4(C)CCCc5sc(N)c(C#N)c54)n3)nc(N3CCN(C)CC3)c2)CC1] |
| Ia-89 | 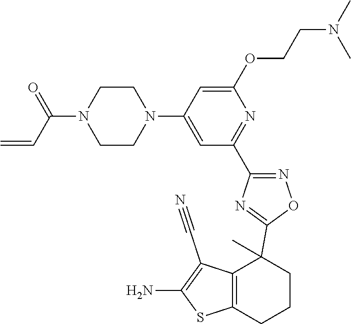 [SMILES: C=CC(=O)N1CCN(c2cc(OCCN(C)C)nc(-c3noc(C4(C)CCCc5sc(N)c(C#N)c54)n3)c2)CC1] |
| Ia-90 | 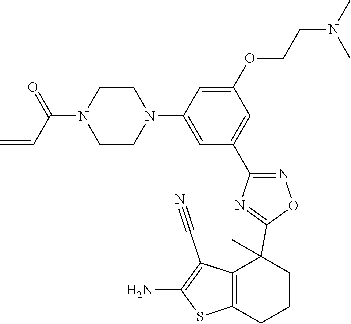 [SMILES: C=CC(=O)N1CCN(c2cc(OCCN(C)C)cc(-c3noc(C4(C)CCCc5sc(N)c(C#N)c54)n3)c2)CC1] |
| Ia-91 | 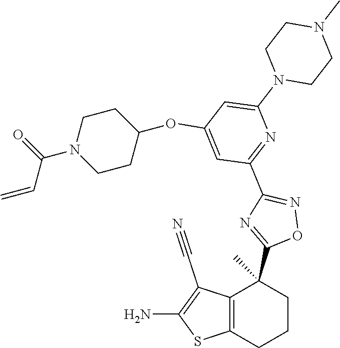 [SMILES: C=CC(=O)N1CCC(Oc2cc(-c3noc([C@@]4(C)CCCc5sc(N)c(C#N)c54)n3)nc(N3CCN(C)CC3)c2)CC1] |
| Ia-92 | 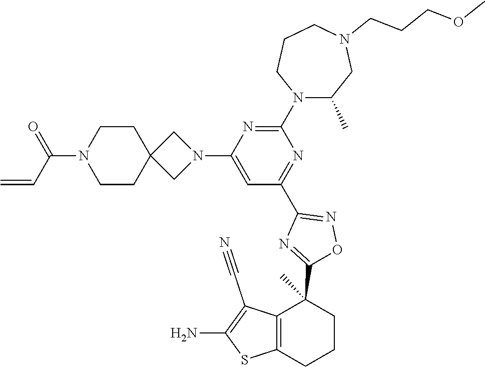 [SMILES: C=CC(=O)N1CCC2(CC1)CN(c1cc(-c3noc([C@@]4(C)CCCc5sc(N)c(C#N)c54)n3)nc(N3CCCN(CCCOC)C[C@@H]3C)n1)C2] |
| Ia-93 | 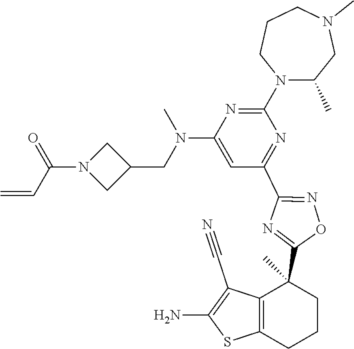 [SMILES: C=CC(=O)N1CC(CN(C)c2cc(-c3noc([C@@]4(C)CCCc5sc(N)c(C#N)c54)n3)nc(N3CCCN(C)C[C@@H]3C)n2)C1] |
| Ia-94 | 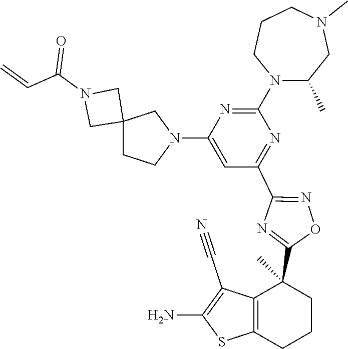 [SMILES: C=CC(=O)N1CC2(CCN(c3cc(-c4noc([C@@]5(C)CCCc6sc(N)c(C#N)c65)n4)nc(N4CCCN(C)C[C@@H]4C)n3)C2)C1] |
| Ia-95 | 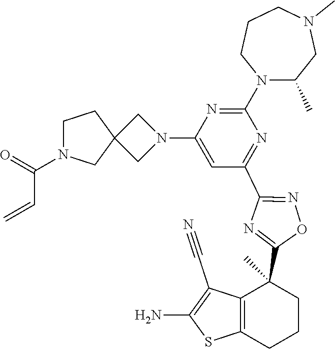 [SMILES: C=CC(=O)N1CCC2(C1)CN(c1cc(-c3noc([C@@]4(C)CCCc5sc(N)c(C#N)c54)n3)nc(N3CCCN(C)C[C@@H]3C)n1)C2] |
| Ia-96 | 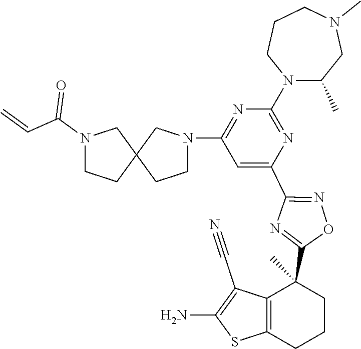 [SMILES: C=CC(=O)N1CCC2(CCN(c3cc(-c4noc([C@@]5(C)CCCc6sc(N)c(C#N)c65)n4)nc(N4CCCN(C)C[C@@H]4C)n3)C2)C1] |
| Ia-97 | 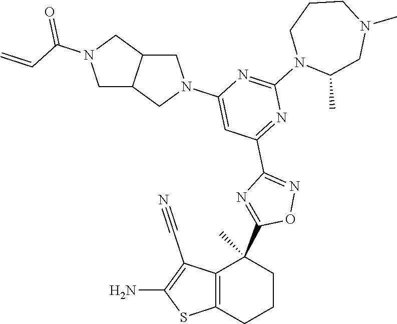 [SMILES: C=CC(=O)N1CC2CN(c3cc(-c4noc([C@@]5(C)CCCc6sc(N)c(C#N)c65)n4)nc(N4CCCN(C)C[C@@H]4C)n3)CC2C1] |
| Ia-98 | 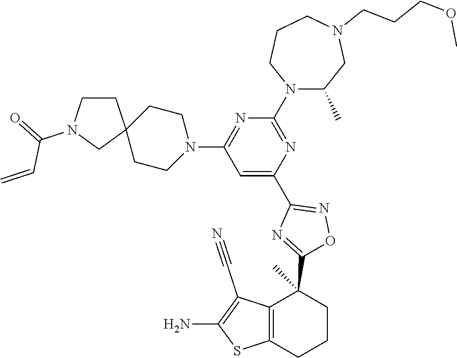 [SMILES: C=CC(=O)N1CCC2(CCN(c3cc(-c4noc([C@@]5(C)CCCc6sc(N)c(C#N)c65)n4)nc(N4CCCN(CCCOC)C[C@@H]4C)n3)CC2)C1] |
| Ia-99 | 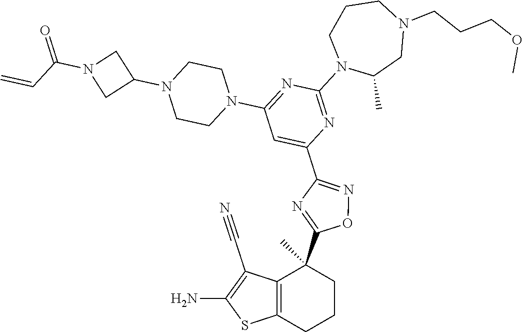 [SMILES: C=CC(=O)N1CC(N2CCN(c3cc(-c4noc([C@@]5(C)CCCc6sc(N)c(C#N)c65)n4)nc(N4CCCN(CCCOC)C[C@@H]4C)n3)CC2)C1] |
| Ia-100 | 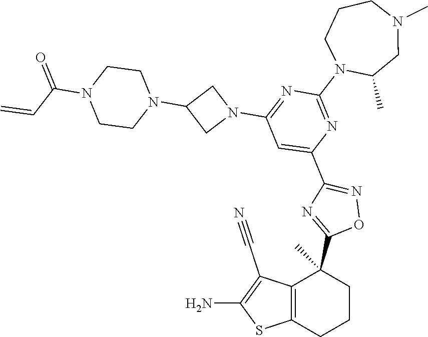 [SMILES: C=CC(=O)N1CCN(C2CN(c3cc(-c4noc([C@@]5(C)CCCc6sc(N)c(C#N)c65)n4)nc(N4CCCN(C)C[C@@H]4C)n3)C2)CC1] |
| Ia-101 | 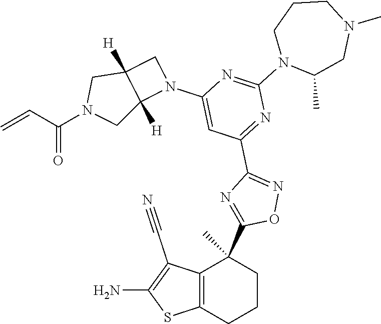 [SMILES: C=CC(=O)N1C[C@@H]2CN(c3cc(-c4noc([C@@]5(C)CCCc6sc(N)c(C#N)c65)n4)nc(N4CCCN(C)C[C@@H]4C)n3)[C@@H]2C1] |
| Ia-102 | 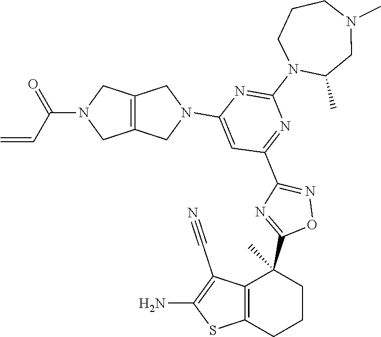 [SMILES: C=CC(=O)N1CC2=C(C1)CN(c1cc(-c3noc([C@@]4(C)CCCc5sc(N)c(C#N)c54)n3)nc(N3CCCN(C)C[C@@H]3C)n1)C2] |
| Ia-103 | 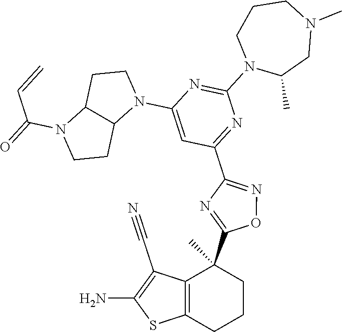 [SMILES: C=CC(=O)N1CCC2C1CCN2c1cc(-c2noc([C@@]3(C)CCCc4sc(N)c(C#N)c43)n2)nc(N2CCCN(C)C[C@@H]2C)n1] |
| Ia-104 | 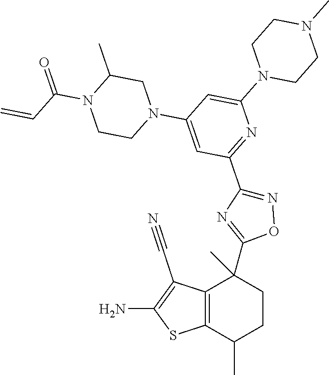 [SMILES: C=CC(=O)N1CCN(c2cc(-c3noc(C4(C)CCC(C)c5sc(N)c(C#N)c54)n3)nc(N3CCN(C)CC3)c2)CC1C] |
| Ia-105 | 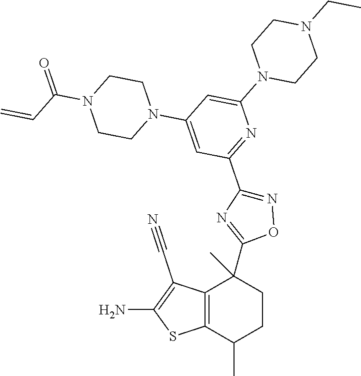 [SMILES: C=CC(=O)N1CCN(c2cc(-c3noc(C4(C)CCC(C)c5sc(N)c(C#N)c54)n3)nc(N3CCN(CC)CC3)c2)CC1] |
| Ia-106 | 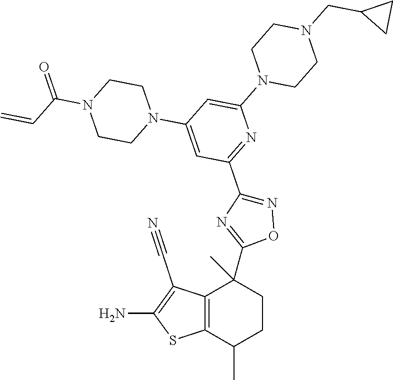 [SMILES: C=CC(=O)N1CCN(c2cc(-c3noc(C4(C)CCC(C)c5sc(N)c(C#N)c54)n3)nc(N3CCN(CC4CC4)CC3)c2)CC1] |
| Ia-107 | 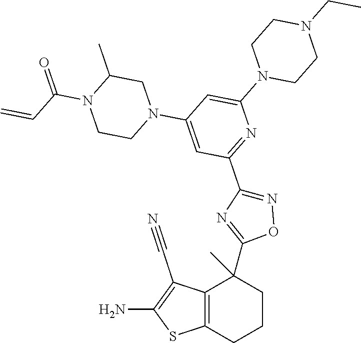 [SMILES: C=CC(=O)N1CCN(c2cc(-c3noc(C4(C)CCCc5sc(N)c(C#N)c54)n3)nc(N3CCN(CC)CC3)c2)CC1C] |
| Ia-108 | 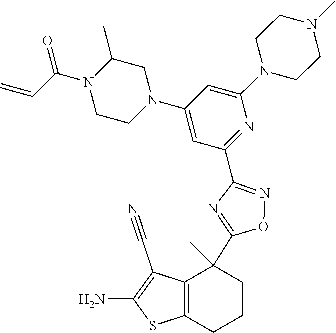 [SMILES: C=CC(=O)N1CCN(c2cc(-c3noc(C4(C)CCCc5sc(N)c(C#N)c54)n3)nc(N3CCN(C)CC3)c2)CC1C] |
| Ia-109 | 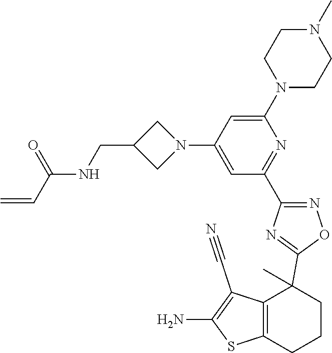 [SMILES: C=CC(=O)NCC1CN(c2cc(-c3noc(C4(C)CCCc5sc(N)c(C#N)c54)n3)nc(N3CCN(C)CC3)c2)C1] |
| Ia-110 | 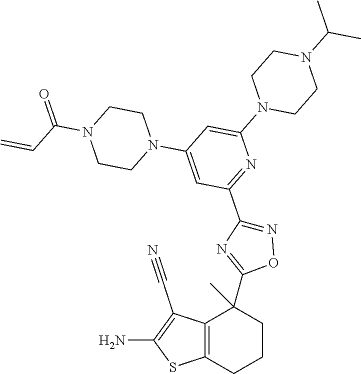 [SMILES: C=CC(=O)N1CCN(c2cc(-c3noc(C4(C)CCCc5sc(N)c(C#N)c54)n3)nc(N3CCN(C(C)C)CC3)c2)CC1] |
| Ia-111 | 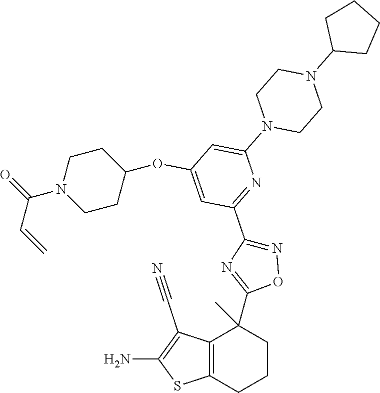 [SMILES: C=CC(=O)N1CCC(Oc2cc(-c3noc(C4(C)CCCc5sc(N)c(C#N)c54)n3)nc(N3CCN(C4CCCC4)CC3)c2)CC1] |
| Ia-112 | 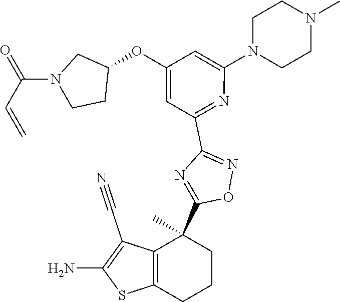 [SMILES: C=CC(=O)N1CC[C@@H](Oc2cc(-c3noc([C@@]4(C)CCCc5sc(N)c(C#N)c54)n3)nc(N3CCN(C)CC3)c2)C1] |
| Ia-113 | 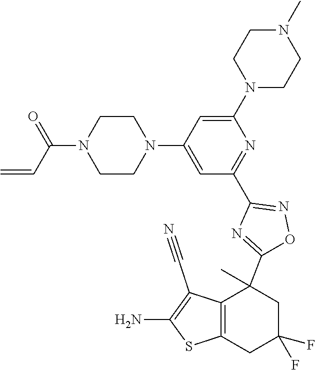 [SMILES: C=CC(=O)N1CCN(c2cc(-c3noc(C4(C)CC(F)(F)Cc5sc(N)c(C#N)c54)n3)nc(N3CCN(C)CC3)c2)CC1] |
| Ia-114 | 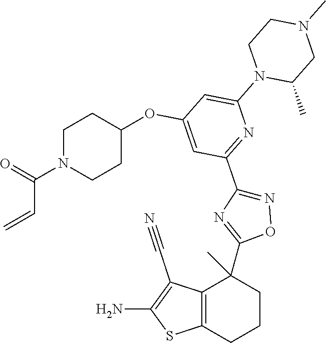 [SMILES: C=CC(=O)N1CCC(Oc2cc(-c3noc(C4(C)CCCc5sc(N)c(C#N)c54)n3)nc(N3CCN(C)C[C@@H]3C)c2)CC1] |
| Ia-115 | 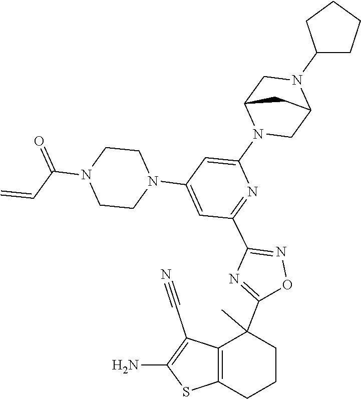 [SMILES: C=CC(=O)N1CCN(c2cc(-c3noc(C4(C)CCCc5sc(N)c(C#N)c54)n3)nc(N3CC4C[C@H]3CN4C3CCCC3)c2)CC1] |
| Ia-116 | 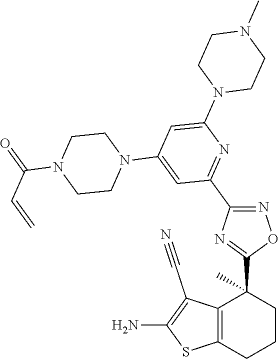 [SMILES: C=CC(=O)N1CCN(c2cc(-c3noc([C@@]4(C)CCCc5sc(N)c(C#N)c54)n3)nc(N3CCN(C)CC3)c2)CC1] |
| Ia-117 | 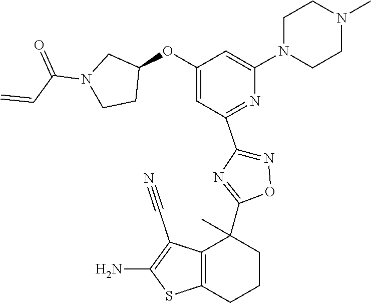 [SMILES: C=CC(=O)N1CC[C@H](Oc2cc(-c3noc(C4(C)CCCc5sc(N)c(C#N)c54)n3)nc(N3CCN(C)CC3)c2)C1] |
| Ia-118 | 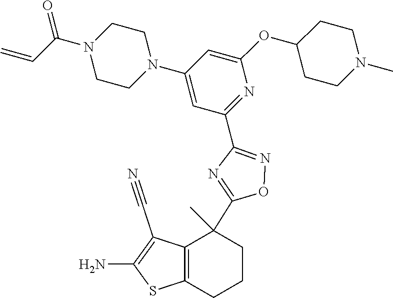 [SMILES: C=CC(=O)N1CCN(c2cc(OC3CCN(C)CC3)nc(-c3noc(C4(C)CCCc5sc(N)c(C#N)c54)n3)c2)CC1] |
| Ia-119 | 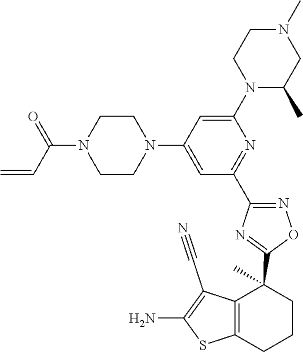 [SMILES: C=CC(=O)N1CCN(c2cc(-c3noc([C@@]4(C)CCCc5sc(N)c(C#N)c54)n3)nc(N3CCN(C)C[C@H]3C)c2)CC1] |
| Ia-120 | 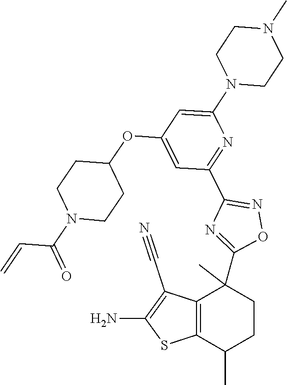 [SMILES: C=CC(=O)N1CCC(Oc2cc(-c3noc(C4(C)CCC(C)c5sc(N)c(C#N)c54)n3)nc(N3CCN(C)CC3)c2)CC1] |
| Ia-121 | 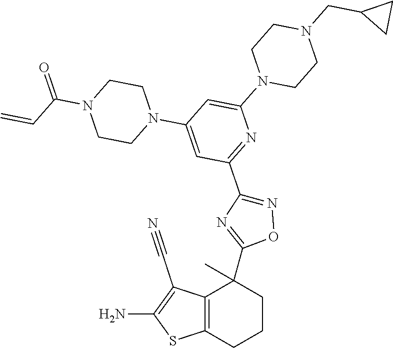 [SMILES: C=CC(=O)N1CCN(c2cc(-c3noc(C4(C)CCCc5sc(N)c(C#N)c54)n3)nc(N3CCN(CC4CC4)CC3)c2)CC1] |
| Ia-122 | 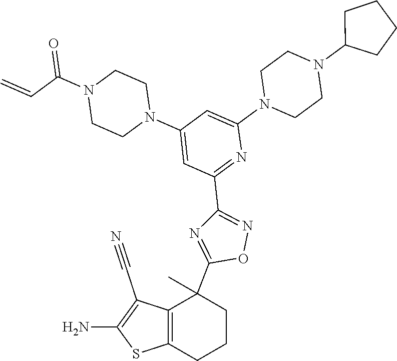 [SMILES: C=CC(=O)N1CCN(c2cc(-c3noc(C4(C)CCCc5sc(N)c(C#N)c54)n3)nc(N3CCN(C4CCCC4)CC3)c2)CC1] |
| Ia-123 | 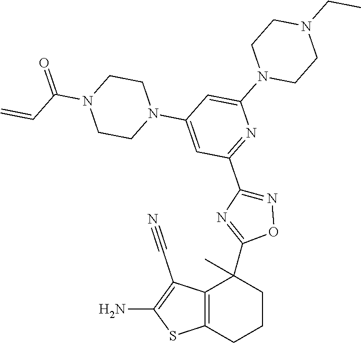 [SMILES: C=CC(=O)N1CCN(c2cc(-c3noc(C4(C)CCCc5sc(N)c(C#N)c54)n3)nc(N3CCN(CC)CC3)c2)CC1] |
| Ia-124 | 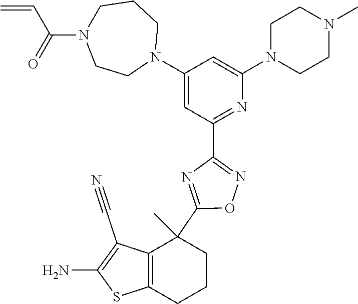 [SMILES: C=CC(=O)N1CCCN(c2cc(-c3noc(C4(C)CCCc5sc(N)c(C#N)c54)n3)nc(N3CCN(C)CC3)c2)CC1] |
| Ia-125 | 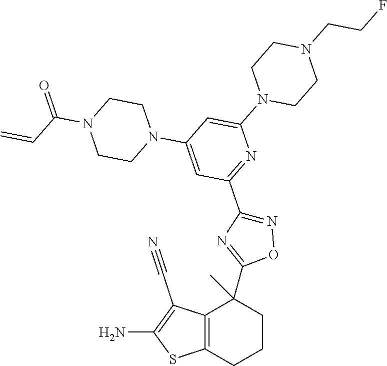 [SMILES: C=CC(=O)N1CCN(c2cc(-c3noc(C4(C)CCCc5sc(N)c(C#N)c54)n3)nc(N3CCN(CCF)CC3)c2)CC1] |
| Ia-126 | 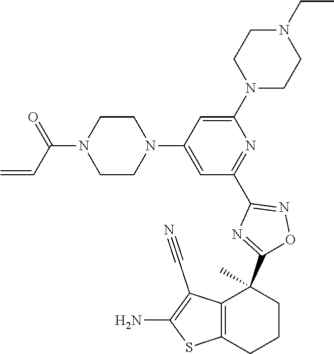 [SMILES: C=CC(=O)N1CCN(c2cc(-c3noc([C@@]4(C)CCCc5sc(N)c(C#N)c54)n3)nc(N3CCN(CC)CC3)c2)CC1] |
| Ia-127 | 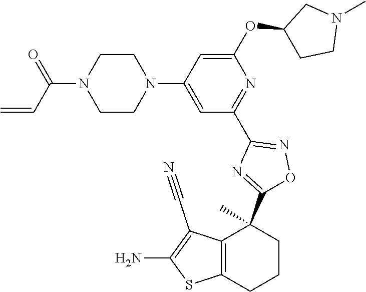 [SMILES: C=CC(=O)N1CCN(c2cc(O[C@@H]3CCN(C)C3)nc(-c3noc([C@@]4(C)CCCc5sc(N)c(C#N)c54)n3)c2)CC1] |
| Ia-128 | 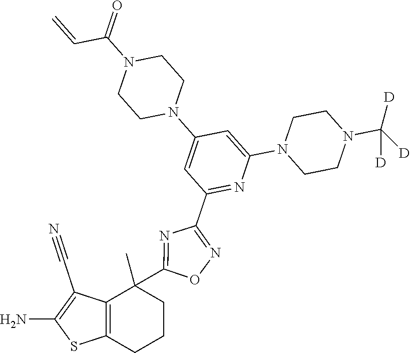 [SMILES: [2H]C([2H])([2H])N1CCN(c2cc(N3CCN(C(=O)C=C)CC3)cc(-c3noc(C4(C)CCCc5sc(N)c(C#N)c54)n3)n2)CC1] |
| Ia-129 | 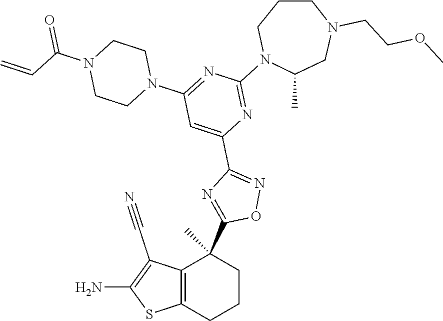 [SMILES: C=CC(=O)N1CCN(c2cc(-c3noc([C@@]4(C)CCCc5sc(N)c(C#N)c54)n3)nc(N3CCCN(CCOC)C[C@@H]3C)n2)CC1] |
| Ia-130 | 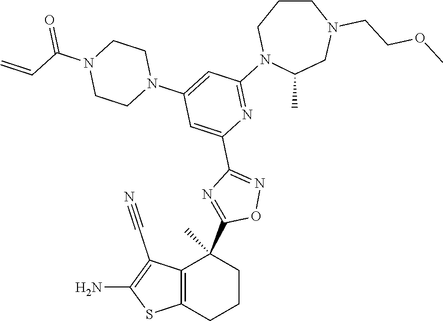 [SMILES: C=CC(=O)N1CCN(c2cc(-c3noc([C@@]4(C)CCCc5sc(N)c(C#N)c54)n3)nc(N3CCCN(CCOC)C[C@@H]3C)c2)CC1] |
| Ia-131 | 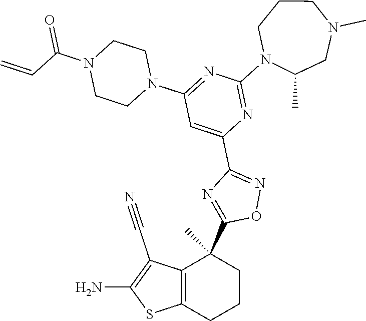 [SMILES: C=CC(=O)N1CCN(c2cc(-c3noc([C@@]4(C)CCCc5sc(N)c(C#N)c54)n3)nc(N3CCCN(C)C[C@@H]3C)n2)CC1] |
| Ia-132 | 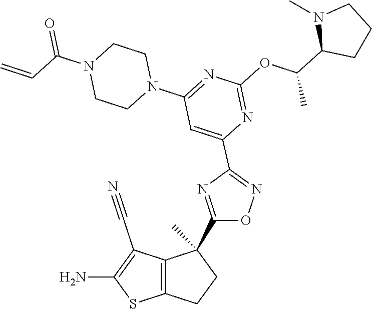 [SMILES: C=CC(=O)N1CCN(c2cc(-c3noc([C@@]4(C)CCc5sc(N)c(C#N)c54)n3)nc(O[C@@H](C)[C@@H]3CCCN3C)n2)CC1] |
| Ia-133 | 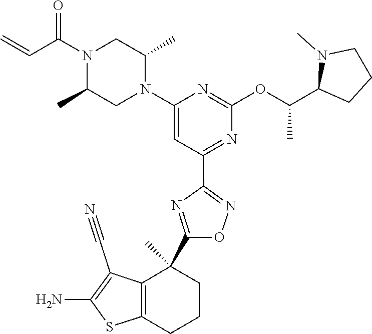 [SMILES: C=CC(=O)N1C[C@H](C)N(c2cc(-c3noc([C@@]4(C)CCCc5sc(N)c(C#N)c54)n3)nc(O[C@@H](C)[C@@H]3CCCN3C)n2)C[C@H]1C] |
| Ia-134 | 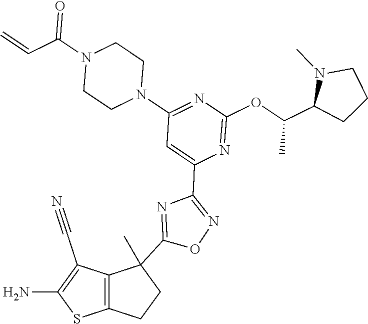 [SMILES: C=CC(=O)N1CCN(c2cc(-c3noc(C4(C)CCc5sc(N)c(C#N)c54)n3)nc(O[C@@H](C)[C@@H]3CCCN3C)n2)CC1] |
| Ia-135 | 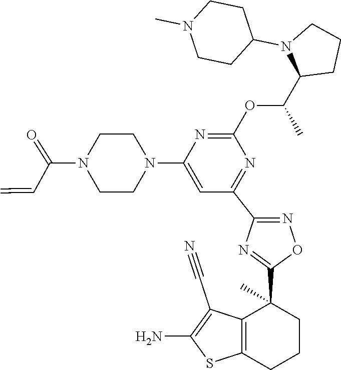 [SMILES: C=CC(=O)N1CCN(c2cc(-c3noc([C@@]4(C)CCCc5sc(N)c(C#N)c54)n3)nc(O[C@@H](C)[C@@H]3CCCN3C3CCN(C)CC3)n2)CC1] |
| Ia-136 | 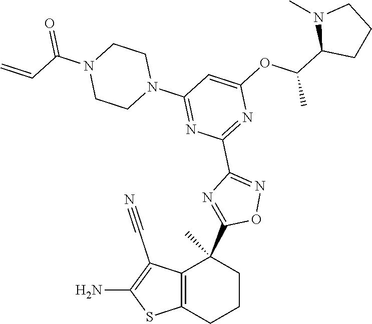 [SMILES: C=CC(=O)N1CCN(c2cc(O[C@@H](C)[C@@H]3CCCN3C)nc(-c3noc([C@@]4(C)CCCc5sc(N)c(C#N)c54)n3)n2)CC1] |
| Ia-137 | 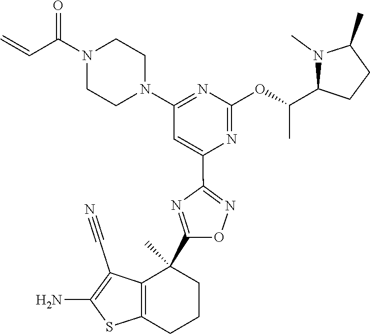 [SMILES: C=CC(=O)N1CCN(c2cc(-c3noc([C@@]4(C)CCCc5sc(N)c(C#N)c54)n3)nc(O[C@@H](C)[C@@H]3CC[C@H](C)N3C)n2)CC1] |
| Ia-138 | 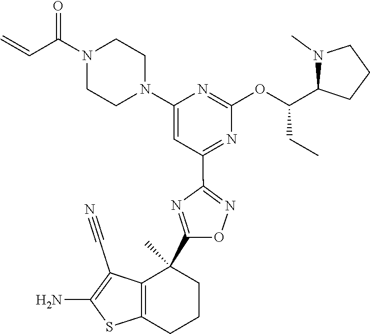 [SMILES: C=CC(=O)N1CCN(c2cc(-c3noc([C@@]4(C)CCCc5sc(N)c(C#N)c54)n3)nc(O[C@@H](CC)[C@@H]3CCCN3C)n2)CC1] |
| Ia-139 | 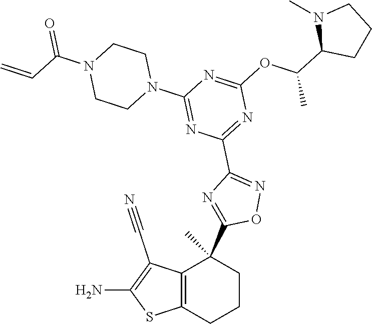 [SMILES: C=CC(=O)N1CCN(c2nc(O[C@@H](C)[C@@H]3CCCN3C)nc(-c3noc([C@@]4(C)CCCc5sc(N)c(C#N)c54)n3)n2)CC1] |
| Ia-140 | 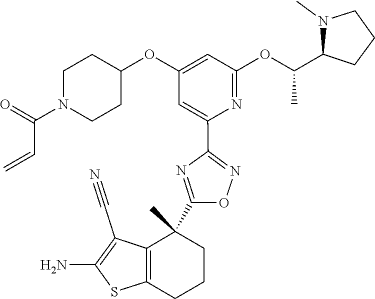 [SMILES: C=CC(=O)N1CCC(Oc2cc(O[C@@H](C)[C@@H]3CCCN3C)nc(-c3noc([C@]4(C)CCCc5sc(N)c(C#N)c54)n3)c2)CC1] |
| Ia-141 | 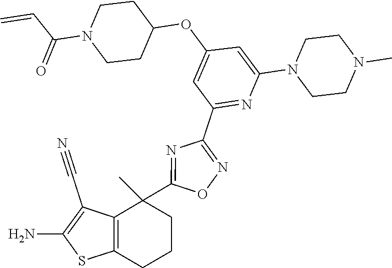 [SMILES: C=CC(=O)N1CCC(Oc2cc(-c3noc(C4(C)CCCc5sc(N)c(C#N)c54)n3)nc(N3CCN(C)CC3)c2)CC1] |
| Ia-142 | 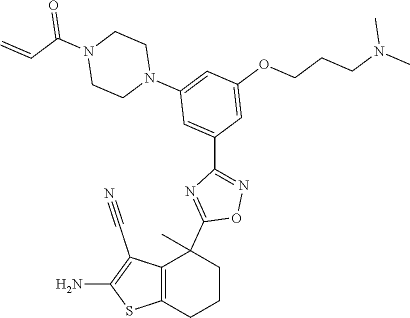 [SMILES: C=CC(=O)N1CCN(c2cc(OCCCN(C)C)cc(-c3noc(C4(C)CCCc5sc(N)c(C#N)c54)n3)c2)CC1] |
| Ia-143 | 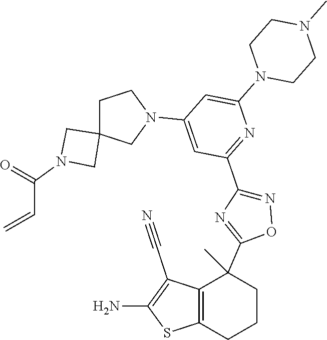 [SMILES: C=CC(=O)N1CC2(CCN(c3cc(-c4noc(C5(C)CCCc6sc(N)c(C#N)c65)n4)nc(N4CCN(C)CC4)c3)C2)C1] |
| Ia-144 | 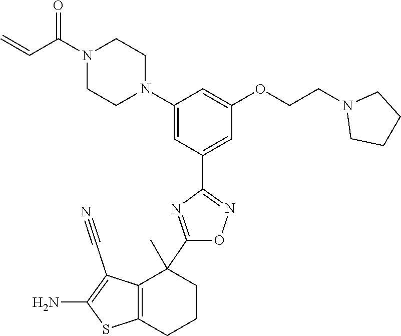 [SMILES: C=CC(=O)N1CCN(c2cc(OCCN3CCCC3)cc(-c3noc(C4(C)CCCc5sc(N)c(C#N)c54)n3)c2)CC1] |
| Ia-145 | 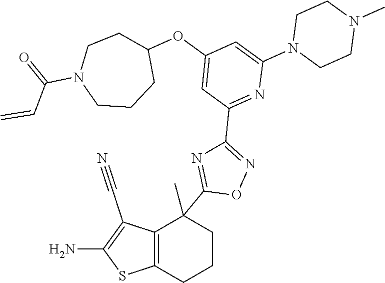 [SMILES: C=CC(=O)N1CCCC(Oc2cc(-c3noc(C4(C)CCCc5sc(N)c(C#N)c54)n3)nc(N3CCN(C)CC3)c2)CC1] |
| Ia-146 | 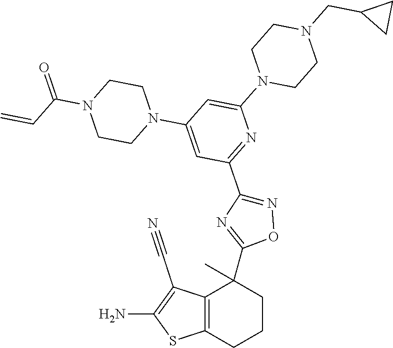 [SMILES: C=CC(=O)N1CCN(c2cc(-c3noc(C4(C)CCCc5sc(N)c(C#N)c54)n3)nc(N3CCN(CC4CC4)CC3)c2)CC1] |
| Ia-147 | 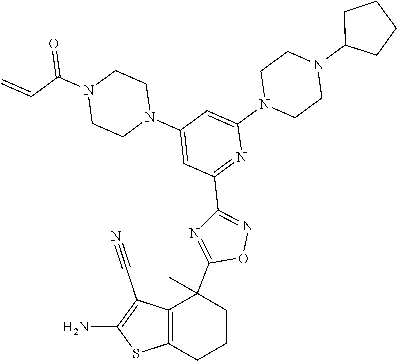 [SMILES: C=CC(=O)N1CCN(c2cc(-c3noc(C4(C)CCCc5sc(N)c(C#N)c54)n3)nc(N3CCN(C4CCCC4)CC3)c2)CC1] |
| Ia-148 | 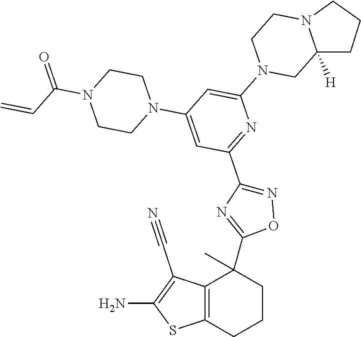 [SMILES: C=CC(=O)N1CCN(c2cc(-c3noc(C4(C)CCCc5sc(N)c(C#N)c54)n3)nc(N3CCN4CCC[C@H]4C3)c2)CC1] |
| Ia-149 | 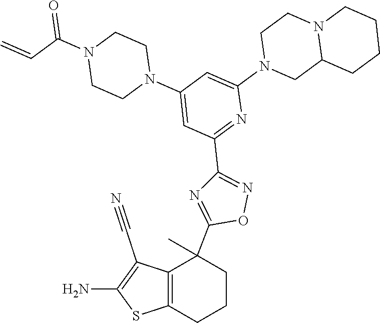 [SMILES: C=CC(=O)N1CCN(c2cc(-c3noc(C4(C)CCCc5sc(N)c(C#N)c54)n3)nc(N3CCN4CCCCC4C3)c2)CC1] |
| Ia-150 | 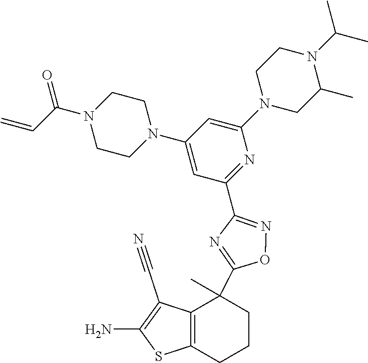 [SMILES: C=CC(=O)N1CCN(c2cc(-c3noc(C4(C)CCCc5sc(N)c(C#N)c54)n3)nc(N3CCN(C(C)C)C(C)C3)c2)CC1] |
| Ia-151 | 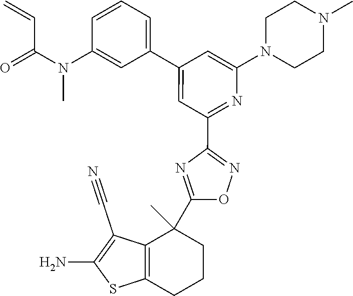 [SMILES: C=CC(=O)N(C)c1cccc(-c2cc(-c3noc(C4(C)CCCc5sc(N)c(C#N)c54)n3)nc(N3CCN(C)CC3)c2)c1] |
| Ia-152 | 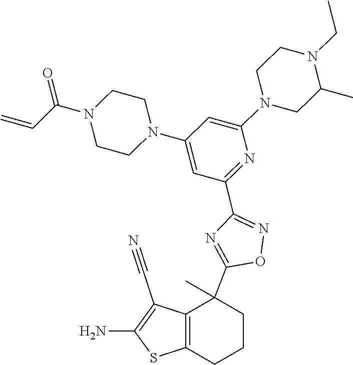 [SMILES: C=CC(=O)N1CCN(c2cc(-c3noc(C4(C)CCCc5sc(N)c(C#N)c54)n3)nc(N3CCN(CC)C(C)C3)c2)CC1] |
| Ia-153 | 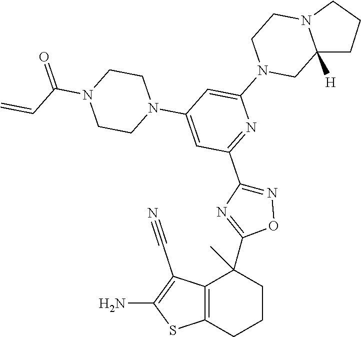 [SMILES: C=CC(=O)N1CCN(c2cc(-c3noc(C4(C)CCCc5sc(N)c(C#N)c54)n3)nc(N3CCN4CCC[C@@H]4C3)c2)CC1] |
| Ia-154 | 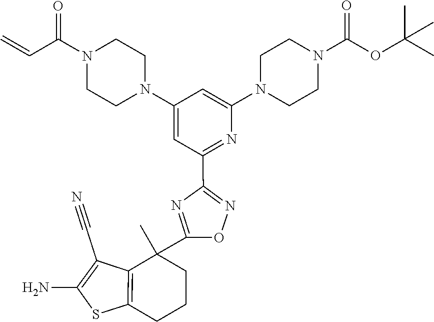 [SMILES: C=CC(=O)N1CCN(c2cc(-c3noc(C4(C)CCCc5sc(N)c(C#N)c54)n3)nc(N3CCN(C(=O)OC(C)(C)C)CC3)c2)CC1] |
| Ia-155 | 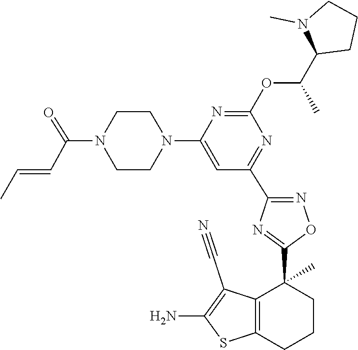 [SMILES: C/C=C/C(=O)N1CCN(c2cc(-c3noc([C@@]4(C)CCCc5sc(N)c(C#N)c54)n3)nc(O[C@@H](C)[C@@H]3CCCN3C)n2)CC1] |
| Ia-156 | 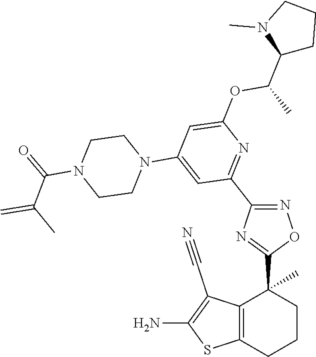 [SMILES: C=C(C)C(=O)N1CCN(c2cc(O[C@@H](C)[C@@H]3CCCN3C)nc(-c3noc([C@@]4(C)CCCc5sc(N)c(C#N)c54)n3)c2)CC1] |
| Ia-156* | 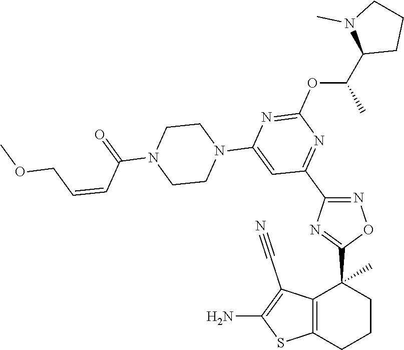 [SMILES: COC/C=C\C(=O)N1CCN(c2cc(-c3noc([C@@]4(C)CCCc5sc(N)c(C#N)c54)n3)nc(O[C@@H](C)[C@@H]3CCCN3C)n2)CC1] |
| Ia-157 | 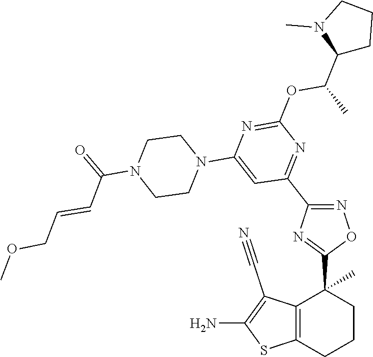 [SMILES: COC/C=C/C(=O)N1CCN(c2cc(-c3noc([C@@]4(C)CCCc5sc(N)c(C#N)c54)n3)nc(O[C@@H](C)[C@@H]3CCCN3C)n2)CC1] |
| Ia-158 | 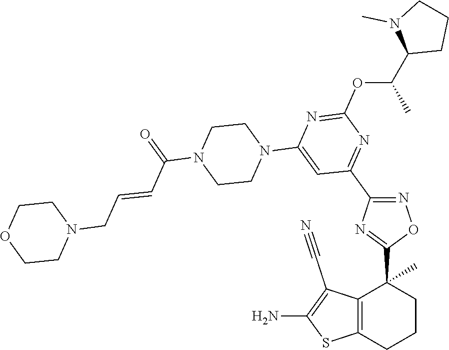 [SMILES: C[C@H](Oc1nc(-c2noc([C@@]3(C)CCCc4sc(N)c(C#N)c43)n2)cc(N2CCN(C(=O)/C=C/CN3CCOCC3)CC2)n1)[C@@H]1CCCN1C] |
| Ia-159 | 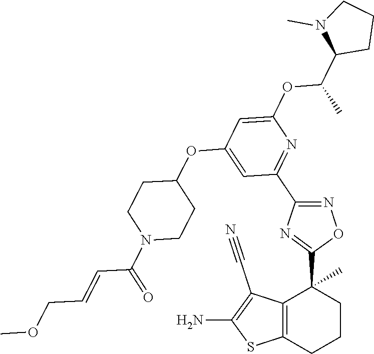 [SMILES: COC/C=C/C(=O)N1CCC(Oc2cc(O[C@@H](C)[C@@H]3CCCN3C)nc(-c3noc([C@@]4(C)CCCc5sc(N)c(C#N)c54)n3)c2)CC1] |
| Ia-160 | 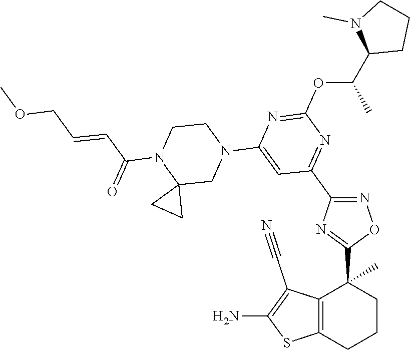 [SMILES: COC/C=C/C(=O)N1CCN(c2cc(-c3noc([C@@]4(C)CCCc5sc(N)c(C#N)c54)n3)nc(O[C@@H](C)[C@@H]3CCCN3C)n2)CC12CC2] |
| Ia-161 | 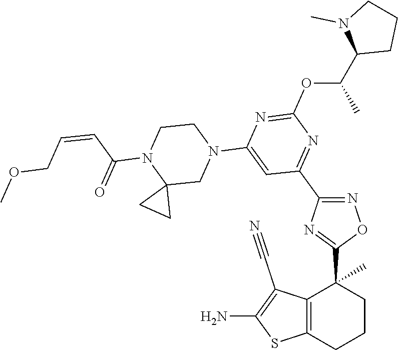 [SMILES: COC/C=C\C(=O)N1CCN(c2cc(-c3noc([C@@]4(C)CCCc5sc(N)c(C#N)c54)n3)nc(O[C@@H](C)[C@@H]3CCCN3C)n2)CC12CC2] |
| Ia-162 | 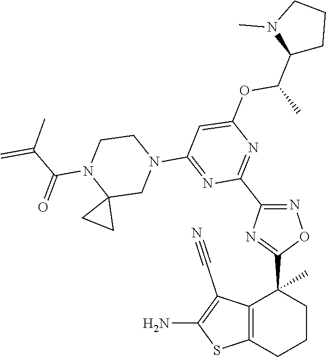 [SMILES: C=C(C)C(=O)N1CCN(c2cc(O[C@@H](C)[C@@H]3CCCN3C)nc(-c3noc([C@@]4(C)CCCc5sc(N)c(C#N)c54)n3)n2)CC12CC2] |
| Ia-163 | 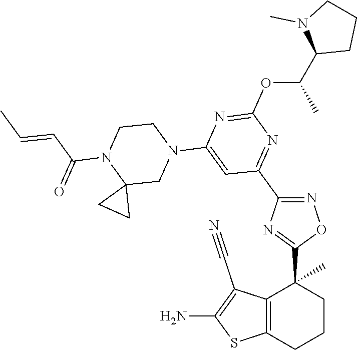 [SMILES: C/C=C/C(=O)N1CCN(c2cc(-c3noc([C@@]4(C)CCCc5sc(N)c(C#N)c54)n3)nc(O[C@@H](C)[C@@H]3CCCN3C)n2)CC12CC2] |
| Ia-164 | 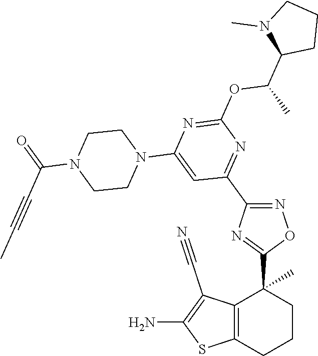 [SMILES: CC#CC(=O)N1CCN(c2cc(-c3noc([C@@]4(C)CCCc5sc(N)c(C#N)c54)n3)nc(O[C@@H](C)[C@@H]3CCCN3C)n2)CC1] |
| Ia-165 | 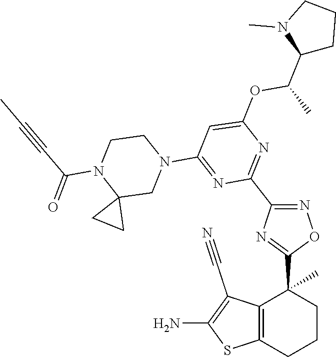 [SMILES: CC#CC(=O)N1CCN(c2cc(O[C@@H](C)[C@@H]3CCCN3C)nc(-c3noc([C@@]4(C)CCCc5sc(N)c(C#N)c54)n3)n2)CC12CC2] |
| Ia-166 | 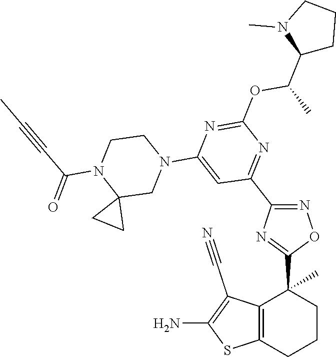 [SMILES: CC#CC(=O)N1CCN(c2cc(-c3noc([C@@]4(C)CCCc5sc(N)c(C#N)c54)n3)nc(O[C@@H](C)[C@@H]3CCCN3C)n2)CC12CC2] |
| Ia-167 | 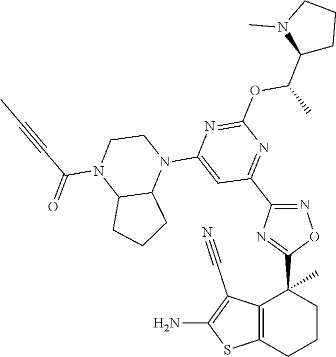 [SMILES: CC#CC(=O)N1CCN(c2cc(-c3noc([C@@]4(C)CCCc5sc(N)c(C#N)c54)n3)nc(O[C@@H](C)[C@@H]3CCCN3C)n2)C2CCCC21] |
| Ia-168 | 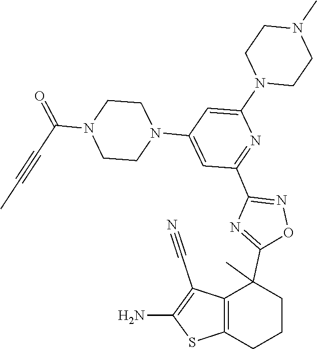 [SMILES: CC#CC(=O)N1CCN(c2cc(-c3noc(C4(C)CCCc5sc(N)c(C#N)c54)n3)nc(N3CCN(C)CC3)c2)CC1] |
| Ia-169 | 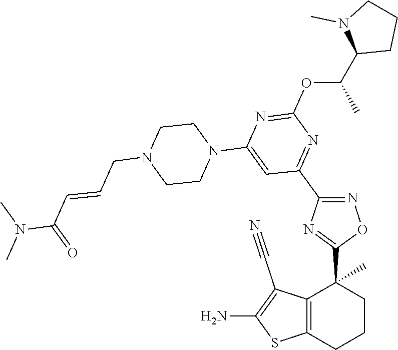 [SMILES: C[C@H](Oc1nc(-c2noc([C@@]3(C)CCCc4sc(N)c(C#N)c43)n2)cc(N2CCN(C/C=C/C(=O)N(C)C)CC2)n1)[C@@H]1CCCN1C] |
| Ia-170 | 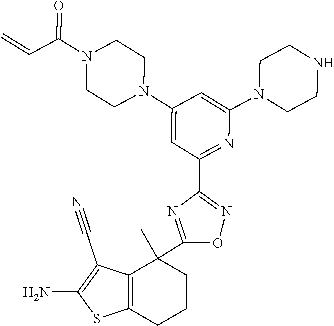 [SMILES: C=CC(=O)N1CCN(c2cc(-c3noc(C4(C)CCCc5sc(N)c(C#N)c54)n3)nc(N3CCNCC3)c2)CC1] |

compounds Ia-1 to Ia-4 and Ib-1 to Ib-9 of WO2023/099612:

| # | structure |
|---|---|
| Ia-1 | 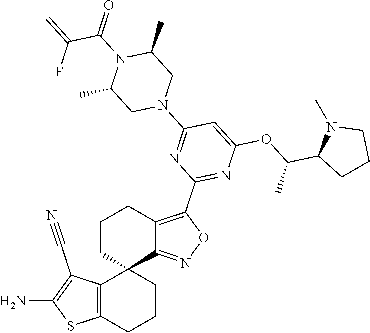 [SMILES: C=C(F)C(=O)N1[C@@H](C)CN(c2cc(O[C@@H](C)[C@@H]3CCCN3C)nc(-c3onc4c3CCC[C@@]43CCCc4sc(N)c(C#N)c43)n2)C[C@@H]1C] |
| Ia-2 | 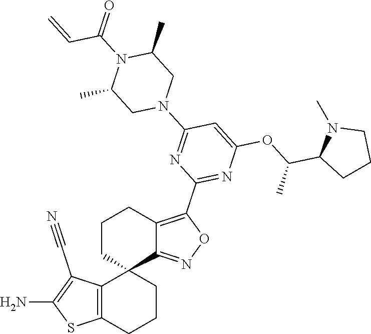 [SMILES: C=CC(=O)N1[C@@H](C)CN(c2cc(O[C@@H](C)[C@@H]3CCCN3C)nc(-c3onc4c3CCC[C@@]43CCCc4sc(N)c(C#N)c43)n2)C[C@@H]1C] |
| Ia-3 | 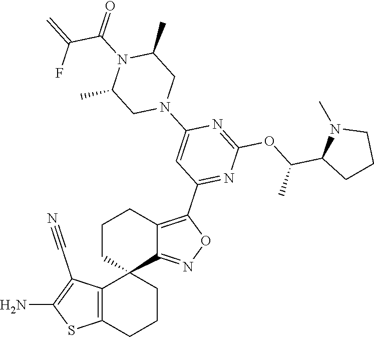 [SMILES: C=C(F)C(=O)N1[C@@H](C)CN(c2cc(-c3onc4c3CCC[C@@]43CCCc4sc(N)c(C#N)c43)nc(O[C@@H](C)[C@@H]3CCCN3C)n2)C[C@@H]1C] |
| Ia-4 | 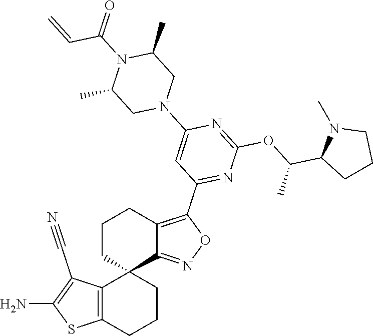 [SMILES: C=CC(=O)N1[C@@H](C)CN(c2cc(-c3onc4c3CCC[C@@]43CCCc4sc(N)c(C#N)c43)nc(O[C@@H](C)[C@@H]3CCCN3C)n2)C[C@@H]1C] |
| Ib-1 | 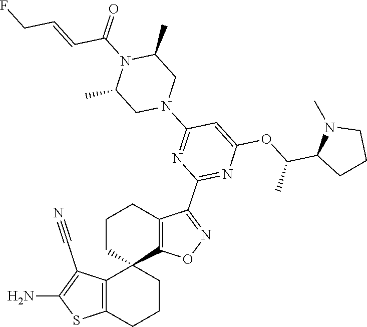 [SMILES: C[C@H](Oc1cc(N2C[C@H](C)N(C(=O)/C=C/CF)[C@@H](C)C2)nc(-c2noc3c2CCC[C@@]32CCCc3sc(N)c(C#N)c32)n1)[C@@H]1CCCN1C] |
| Ib-2 | 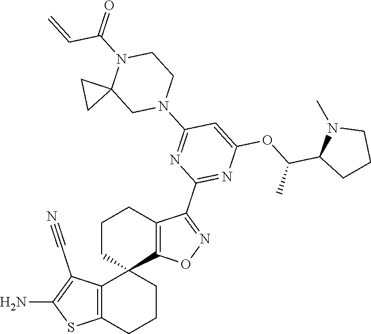 [SMILES: C=CC(=O)N1CCN(c2cc(O[C@@H](C)[C@@H]3CCCN3C)nc(-c3noc4c3CCC[C@@]43CCCc4sc(N)c(C#N)c43)n2)CC12CC2] |
| Ib-3 | 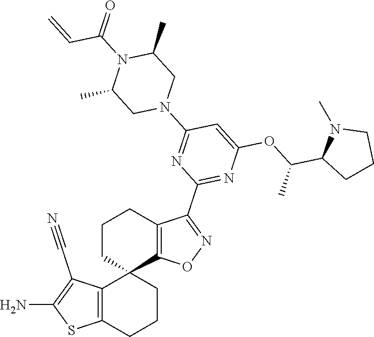 [SMILES: C=CC(=O)N1[C@@H](C)CN(c2cc(O[C@@H](C)[C@@H]3CCCN3C)nc(-c3noc4c3CCC[C@@]43CCCc4sc(N)c(C#N)c43)n2)C[C@@H]1C] |
| Ib-4 | 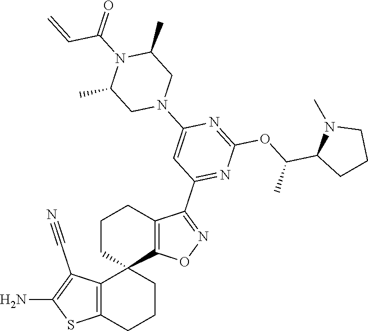 [SMILES: C=CC(=O)N1[C@@H](C)CN(c2cc(-c3noc4c3CCC[C@@]43CCCc4sc(N)c(C#N)c43)nc(O[C@@H](C)[C@@H]3CCCN3C)n2)C[C@@H]1C] |
| Ib-5 | 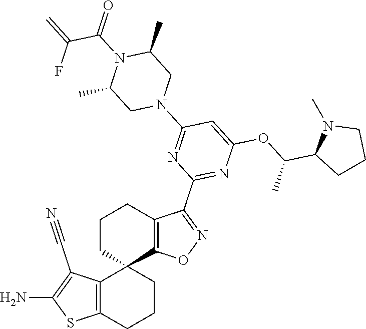 [SMILES: C=C(F)C(=O)N1[C@@H](C)CN(c2cc(O[C@@H](C)[C@@H]3CCCN3C)nc(-c3noc4c3CCC[C@@]43CCCc4sc(N)c(C#N)c43)n2)C[C@@H]1C] |
| Ib-6 | 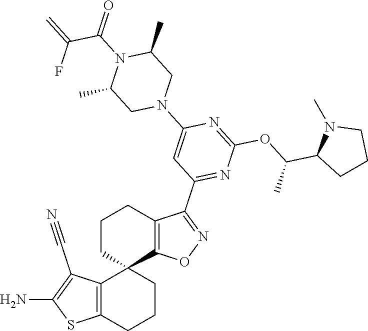 [SMILES: C=C(F)C(=O)N1[C@@H](C)CN(c2cc(-c3noc4c3CCC[C@@]43CCCc4sc(N)c(C#N)c43)nc(O[C@@H](C)[C@@H]3CCCN3C)n2)C[C@@H]1C] |
| Ib-7 | 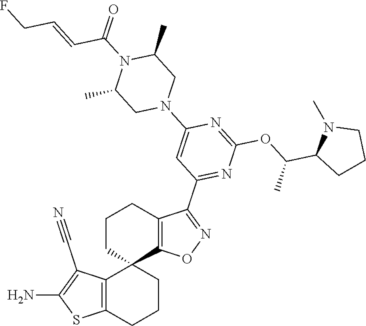 [SMILES: C[C@H](Oc1nc(-c2noc3c2CCC[C@@]32CCCc3sc(N)c(C#N)c32)cc(N2C[C@H](C)N(C(=O)/C=C/CF)[C@@H](C)C2)n1)[C@@H]1CCCN1C] |
| Ib-8 | 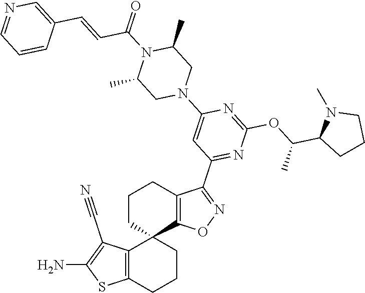 [SMILES: C[C@H](Oc1nc(-c2noc3c2CCC[C@@]32CCCc3sc(N)c(C#N)c32)cc(N2C[C@H](C)N(C(=O)/C=C/c3cccnc3)[C@@H](C)C2)n1)[C@@H]1CCCN1C] |
| Ib-9 | 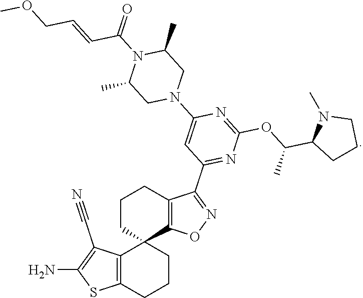 [SMILES: COC/C=C/C(=O)N1[C@@H](C)CN(c2cc(-c3noc4c3CCC[C@@]43CCCc4sc(N)c(C#N)c43)nc(O[C@@H](C)[C@@H]3CCCN3C)n2)C[C@@H]1C] |

Further examples of KRAS G12C inhibitors are compounds known as: divarasib (GDC-6036), opnurasib (also known as JDQ443), garsorasib (D-1553), glecirasib (JAC-21822), GFH925/GF105/IBI351, RMC-6291, LY3537982, JNJ-74699157 and LY3499446.

In an aspect, the KRAS G12C inhibitor is selected from the group consisting of: sotorasib, adagrasib, compounds Ib-1 to Ib-16, Ic-1 to Ic-9, Id-1 to Id-9 and Ie-1 of WO2021/245051, compounds Ia-1 to Ia-170 of WO2021/245055, compounds Ia-1 to Ia-4 and Ib-1 to Ib-9 of WO2023/099612, divarasib, opnurasib, garsorasib, glecirasib, IBI351, RMC-6291, LY3537982, JNJ-74699157 and LY3499446. In an aspect, the KRAS G12C inhibitor is sotorasib or adagrasib.

In an aspect, the KRAS G12C inhibitor is selected from the group consisting of: compounds Ib-1 to Ib-16, Ic-1 to Ic-9, Id-1 to Id-9 and Ie-1 of WO2021/245051, compounds Ia-1 to Ia-170 of WO2021/245055 and compounds Ia-1 to Ia-4 and Ib-1 to Ib-9 of WO2023/099612. The combination of all aspect in respect of the nature of zongertinib or the pharmaceutically acceptable salt thereof as described herein with all aspects in respect of the nature of the KRAS G12C inhibitor as described herein results in specific combinations which shall all be deemed to be specifically disclosed and to be embodiments of the invention and of all combinations, compositions, kits, methods, uses and compounds for use described herein. Preferred embodiments are combinations of aspects of zongertinib or the pharmaceutically acceptable salt thereof in respect of the nature of zongertinib with aspects of sotorasib or adagrasib in respect of the nature of the KRAS G12C inhibitor.

### Dosing Schedule

In another aspect, zongertinib or the pharmaceutically acceptable salt thereof is administered daily.

In another aspect, zongertinib or the pharmaceutically acceptable salt thereof is administered orally, preferably as a tablet.

In another aspect, zongertinib or the pharmaceutically acceptable salt thereof is administered daily and orally, preferably as a tablet.

In another aspect, zongertinib or the pharmaceutically acceptable salt thereof is administered in a daily dose of at least 30 mg, preferably of at least 60 mg, preferably of at least 80 mg, preferably of at least 120 mg, preferably of at least 180 mg, preferably of at least 200 mg, preferably of at least 240 mg, preferably of at least 300 mg, preferably of at least 360 mg.

In another aspect, zongertinib or the pharmaceutically acceptable salt thereof is administered in a daily dose of at least 30 mg, preferably of at least 60 mg, preferably of at least 80 mg, preferably of at least 120 mg, preferably of at least 180 mg, preferably of at least 200 mg, preferably of at least 240 mg, preferably of at least 300 mg.

As used herein, “daily” means within a timeframe of 24 h. The expressions “daily dose” and “total daily dose” refer to the amount of active substance, i.e. zongertinib or the KRAS G12C inhibitor, which is administered within said timeframe of 24 h. The 24 h timeframe does not necessarily start at noon or midnight. Preferably, the daily dose is administered once or twice per day.

In another aspect, zongertinib or the pharmaceutically acceptable salt thereof is administered in a daily dose of 30 mg to 600 mg, preferably of 60 mg to 600 mg, preferably of 80 mg to 600 mg, preferably of 120 mg to 600 mg.

In another aspect, zongertinib or the pharmaceutically acceptable salt thereof is administered in a daily dose of 30 mg to 360 mg, preferably of 60 mg to 360 mg, preferably of 80 mg to 360 mg, preferably of 120 mg to 360 mg.

In another aspect, zongertinib or the pharmaceutically acceptable salt thereof is administered in a daily dose of 30 mg to 300 mg, preferably of 60 mg to 300 mg, preferably of 80 mg to 300 mg, preferably of 120 mg to 300 mg.

In another aspect, zongertinib or the pharmaceutically acceptable salt thereof is administered in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg.

In another aspect, zongertinib or the pharmaceutically acceptable salt thereof is administered in a daily dose of 30 mg, 60 mg, 120 mg, 180 mg, 200 mg, 240 mg, 300 mg or 360 mg.

In another aspect, zongertinib or the pharmaceutically acceptable salt thereof is administered in a daily dose of 30 mg, 60 mg, 120 mg, 180 mg, 200 mg, 240 mg or 300 mg.

In another aspect, zongertinib or the pharmaceutically acceptable salt thereof is administered in a daily dose of 60 mg.

In another aspect, zongertinib or the pharmaceutically acceptable salt thereof is administered in a daily dose of 120 mg.

In another aspect, zongertinib or the pharmaceutically acceptable salt thereof is administered in a daily dose of 180 mg.

In another aspect, zongertinib or the pharmaceutically acceptable salt thereof is administered in a daily dose of 200 mg.

In another aspect, zongertinib or the pharmaceutically acceptable salt thereof is administered in a daily dose of 240 mg.

In another aspect, zongertinib or the pharmaceutically acceptable salt thereof is administered in a daily dose of 300 mg.

In another aspect, zongertinib or the pharmaceutically acceptable salt thereof is administered in a daily dose of 360 mg.

In another aspect, zongertinib or the pharmaceutically acceptable salt thereof is administered once or twice daily. This means that the daily dose, in particular the daily dose as defined in any aspect described herein, is administered either as a single dose (once daily) or is divided in two separate administrations, each administered at a different time point of the day (twice daily), i.e. there are two administrations within 24 h. In case of a twice daily administration, it is preferred that each of the two daily administrations of zongertinib corresponds to half the daily dose. When zongertinib is administered twice daily, the two separate administrations are preferably separated by a time interval of at least 1, 2, 3, 4, 5, 6, 7, 8, 9, 10, 11 or 12 hours, preferably of 4, 5, 6, 7, 8, 9, 10, 11 or 12 hours, still preferably of 8, 9, 10, 11 or 12 hours, also preferably of approximately 12 hours.

In another aspect, zongertinib or the pharmaceutically acceptable salt thereof is administered once or twice daily and orally, preferably as a tablet.

In another aspect, zongertinib or the pharmaceutically acceptable salt thereof is administered at least for 21 consecutive days. In further preferred embodiments, zongertinib or the pharmaceutically acceptable salt thereof is administered for 21 days multiplied by X, wherein X is a natural number equal or larger than 1.

In another aspect, zongertinib or the pharmaceutically acceptable salt thereof is administered once daily in a daily dose of 60 mg, 120 mg, 180 mg, 240 mg, 300 mg, 360 mg, 400 mg, 420 mg, 480 mg, 500 mg, 540 mg or 600 mg or zongertinib or the pharmaceutically acceptable salt thereof is administered twice daily in a daily dose of 30 mg, 60 mg, 120 mg, 200 mg, 300 mg, 360 mg, 400 mg, 420 mg, 480 mg, 500 mg, 540 mg or 600 mg.

In another aspect, zongertinib or the pharmaceutically acceptable salt thereof is administered once daily in a daily dose of 60 mg, 120 mg, 180 mg, 240 mg, 300 mg, 360 mg or zongertinib or the pharmaceutically acceptable salt thereof is administered twice daily in a daily dose of 30 mg, 60 mg, 120 mg, 200 mg, 300 mg or 360 mg.

In another aspect, zongertinib or the pharmaceutically acceptable salt thereof is administered once daily in a daily dose of 60 mg, 120 mg, 180 mg, 240 mg or 300 mg or zongertinib or the pharmaceutically acceptable salt thereof is administered twice daily in a daily dose of 30 mg, 60 mg, 120 mg, 200 mg or 300 mg.

In another aspect, zongertinib or the pharmaceutically acceptable salt thereof is administered once daily in a daily dose of 60 mg.

In another aspect, zongertinib or the pharmaceutically acceptable salt thereof is administered once daily in a daily dose of 120 mg.

In another aspect, zongertinib or the pharmaceutically acceptable salt thereof is administered once daily in a daily dose of 180 mg.

In another aspect, zongertinib or the pharmaceutically acceptable salt thereof is administered once daily in a daily dose of 200 mg.

In another aspect, zongertinib or the pharmaceutically acceptable salt thereof is administered once daily in a daily dose of 240 mg.

In another aspect, zongertinib or the pharmaceutically acceptable salt thereof is administered once daily in a daily dose of 300 mg.

In another aspect, zongertinib or the pharmaceutically acceptable salt thereof is administered once daily in a daily dose of 360 mg.

In another aspect, zongertinib or the pharmaceutically acceptable salt thereof is administered orally, preferably as a tablet, once daily in a daily dose of 60 mg, 120 mg, 180 mg, 240 mg, 300 mg, 360 mg, 400 mg, 420 mg, 480 mg, 500 mg, 540 mg or 600 mg or zongertinib or the pharmaceutically acceptable salt thereof is administered orally, preferably as a tablet, twice daily in a daily dose of 30 mg, 60 mg, 120 mg, 200 mg, 300 mg, 360 mg, 400 mg, 420 mg, 480 mg, 500 mg, 540 mg or 600 mg.

In another aspect, zongertinib or the pharmaceutically acceptable salt thereof is administered orally, preferably as a tablet, once daily in a daily dose of 60 mg, 120 mg, 180 mg, 240 mg, 300 mg or 360 mg or zongertinib or the pharmaceutically acceptable salt thereof is administered orally, preferably as a tablet, twice daily in a daily dose of 30 mg, 60 mg, 120 mg, 200 mg, 300 mg or 360 mg.

In another aspect, zongertinib or the pharmaceutically acceptable salt thereof is administered orally, preferably as a tablet, once daily in a daily dose of 60 mg, 120 mg, 180 mg, 240 mg or 300 mg or zongertinib or the pharmaceutically acceptable salt thereof is administered orally, preferably as a tablet, twice daily in a daily dose of 30 mg, 60 mg, 120 mg, 200 mg or 300 mg.

In another aspect, zongertinib or the pharmaceutically acceptable salt thereof is administered orally, preferably as a tablet, once daily in a daily dose of 60 mg.

In another aspect, zongertinib or the pharmaceutically acceptable salt thereof is administered orally, preferably as a tablet, once daily in a daily dose of 120 mg.

In another aspect, zongertinib or the pharmaceutically acceptable salt thereof is administered orally, preferably as a tablet, once daily in a daily dose of 180 mg.

In another aspect, zongertinib or the pharmaceutically acceptable salt thereof is administered orally, preferably as a tablet, once daily in a daily dose of 200 mg.

In another aspect, zongertinib or the pharmaceutically acceptable salt thereof is administered orally, preferably as a tablet, once daily in a daily dose of 240 mg.

In another aspect, zongertinib or the pharmaceutically acceptable salt thereof is administered orally, preferably as a tablet, once daily in a daily dose of 300 mg.

In another aspect, zongertinib or the pharmaceutically acceptable salt thereof is administered orally, preferably as a tablet, once daily in a daily dose of 360 mg.

It is also possible to combine any of the above defined doses of zongertinib within the course of a treatment, for example by altering the daily dose. For instance, it is possible to increase or decrease a dose of 120 mg (once or twice daily) for subsequent administrations.

In another aspect, the KRAS G12C inhibitor is administered daily.

In another aspect, the KRAS G12C inhibitor is administered orally, preferably as a tablet.

In another aspect, the KRAS G12C inhibitor is administered in a daily dose of at least 100 mg, preferably of at least 200 mg, preferably of at least 300 mg, preferably of at least 400 mg, preferably of at least 500 mg, preferably of at least 600 mg, preferably of at least 700 mg, preferably of at least 800 mg, preferably of at least 900 mg, preferably of at least 1000 mg, preferably of at least 1100 mg, preferably of at least 1200 mg, preferably of at least 1300 mg, preferably of at least 1500 mg.

In another aspect, the KRAS G12C inhibitor is administered in a daily dose of 100 mg to 1500 mg, preferably of 500 mg to 1400 mg, preferably of 600 mg to 1300 mg.

In another aspect, the KRAS G12C inhibitor is administered in a daily dose of 100 mg, 200 mg, 300 mg, 400 mg, 500 mg, 600 mg, 700 mg, 800 mg, 900 mg or 1000 mg.

In another aspect, the KRAS G12C inhibitor is administered once or twice daily. In this aspect, the KRAS G12C inhibitor is preferably administered at the daily dose as defined in any of the previous aspects.

In another aspect, the KRAS G12C inhibitor is administered once or twice daily and orally, preferably as a tablet. In this aspect, the KRAS G12C inhibitor is preferably administered at the daily dose as defined in any of the previous aspects.

Adagrasib, sotorasib and any other approved KRAS G12C inhibitor may be administered according to the respective Label or Summary of Product Characteristics.

Any aspect referring to the administration of zongertinib or the pharmaceutically acceptable salt thereof (e.g. referring to its daily dose, oral administration route, once or twice daily schedule, etc.) may be combined with any aspect referring to the KRAS G12C inhibitor (e.g. referring to its dose, administration route, posology, etc.) to provide further aspects of the invention.

### Combination Therapy

It is to be understood that the combinations, compositions, kits, methods, uses or compounds for use according to this invention may envisage the simultaneous, concurrent, sequential, successive, alternate or separate administration of the active agents or components. It will be appreciated that zongertinib or the pharmaceutically acceptable salt thereof and the KRAS G12C inhibitor can be formulated either together or independently. For example, zongertinib or the pharmaceutically acceptable salt thereof and the KRAS G12C inhibitor may be administered either as part of the same pharmaceutical composition/dosage form or, preferably, in separate pharmaceutical compositions/dosage forms.

Zongertinib or the pharmaceutically acceptable salt thereof and the KRAS G12C inhibitor thus may be formulated, alone or together, in suitable dosage unit formulations containing conventional non-toxic pharmaceutically acceptable carriers, excipients and/or vehicles appropriate for each route of administration. Typical pharmaceutical compositions for administering zongertinib or the pharmaceutically acceptable salt thereof and the KRAS G12C inhibitor, separately or jointly, include for example tablets, capsules, suppositories, solutions, e.g. solutions for injection and infusion, elixirs, emulsions or dispersible powders. Dosage forms and formulations of active ingredients are known in the art and further described herein. Preferably, zongertinib is administered in the form described in WO 2024/133289, which is herein incorporated by reference in its entirety. Also preferably, adagrasib, sotorasib or any other approved KRAS G12C inhibitor is administered in the form described in the respective Label or Summary of Product Characteristics.

As used herein, “combination” or “combined” and grammatical variants thereof within the meaning of this invention include, but are not limited to, a product, product for use, use or method that results from the mixing or combining of more than one active agent, in particular zongertinib and the KRAS G12C inhibitor as defined herein. The expressions “combination”, “combined” and grammatical variants thereof comprise both fixed (e.g. pharmaceutical composition) and non-fixed (e.g. free) combinations (e.g. kits), products, products for use, uses and methods, such as e.g. the simultaneous, concurrent, sequential, successive, alternate or separate use of zongertinib and the KRAS G12C inhibitor as defined herein.

As used herein, “combination” or “combined” and grammatical variants thereof refer in particular to a combination that takes place within the same line of treatment. Preferably, in the products, products for use, uses and methods described herein, there is at least one day where both zongertinib, or a pharmaceutically acceptable salt thereof, and the KRAS G12C inhibitor are administered.

The term “fixed combination” means that zongertinib and the KRAS G12C inhibitor as defined herein are both administered to a patient simultaneously in the form of a single entity or dosage form. The term “non-fixed combination” means that the active agents are both administered to a patient as separate entities or dosage forms either simultaneously, concurrently, sequentially, successively, alternatively or otherwise separately with no specific time limits.

The term “simultaneous” refers to the administration of both compounds/compositions at substantially the same time. This form of administration may also be referred to as “concomitant” administration. The term “concurrent” refers to administration of the active ingredients within the same general time period, for example on the same day(s) but not necessarily at the same time. The term “sequential” administration includes administration of one active ingredient during a first time period, for example over the course of a few hours, days or a week, using one or more doses, followed by administration of the other active ingredient during a second time period, for example over the course of a few hours, days or a week, using one or more doses. An overlapping schedule may also be employed, which includes administration of the active ingredients on different days over the treatment period, not necessarily according to a regular sequence. The term “successive” administration, alternatively, refers to an administration where the second administration step is carried out immediately once the administration of the first compounds has been finished. Alternate administration includes administration of one active ingredient during a time period, for example over the course of a few hours, days or a week, followed by administration of the other active ingredient during a subsequent period of time, for example over the course of a few hours, days or a week, and then repeating the pattern for one or more cycles, wherein the overall number of repeats depends on the chosen dosage regimen. Variations of these general administration forms may also be employed.

Accordingly, in another aspect, zongertinib or the pharmaceutically acceptable salt thereof and the KRAS G12C inhibitor are administered simultaneously, concurrently, sequentially, successively, alternately or separately.

Preferably, the combination of zongertinib or the pharmaceutically acceptable salt thereof and the KRAS G12C inhibitor is administered simultaneously or sequentially, such that the concentration of the individual active ingredients in the body is high enough to exhibit a synergistic effect. Combination therapy according to the invention can occur with or without instructions for combined use. The two or more active ingredients may thus be administered entirely separately or be entirely separate pharmaceutical dosage forms. The individual active ingredients may be pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active. It can refer to either a fixed combination in one dosage unit form, or a kit of parts for the combined administration where zongertinib or the pharmaceutically acceptable salt thereof and the KRAS G12C inhibitor may be administered independently at the same time or separately within time intervals, especially where these time intervals allow that the KRAS G12C inhibitor show a cooperative (synergistic) effect.

In another aspect, zongertinib or the pharmaceutically acceptable salt thereof and the KRAS G12C inhibitor are administered simultaneously.

In another aspect, zongertinib or the pharmaceutically acceptable salt thereof and the KRAS G12C inhibitor are administered concurrently.

In another aspect, zongertinib or the pharmaceutically acceptable salt thereof and the KRAS G12C inhibitor are administered sequentially.

In another aspect, zongertinib or the pharmaceutically acceptable salt thereof and the KRAS G12C inhibitor are administered successively.

In another aspect, zongertinib or the pharmaceutically acceptable salt thereof and the KRAS G12C inhibitor are administered alternately.

In another aspect, zongertinib or the pharmaceutically acceptable salt thereof and the KRAS G12C inhibitor are administered separately.

In another aspect, zongertinib or the pharmaceutically acceptable salt thereof is administered before the KRAS G12C inhibitor. Preferably, the treatment comprises one day when both zongertinib or the pharmaceutically acceptable salt thereof and the KRAS G12C inhibitor are administered and zongertinib or the pharmaceutically acceptable salt thereof is administered before the KRAS G12C inhibitor.

In another aspect, the KRAS G12C inhibitor is administered immediately after zongertinib or the pharmaceutically acceptable salt thereof. In this context, “immediately after” means, e.g. 30 minutes, 1 hour, 2 hours, 3 hours or 4 hours after.

In another aspect, zongertinib or the pharmaceutically acceptable salt thereof is administered after the KRAS G12C inhibitor. Preferably, the treatment comprises one day when both zongertinib or the pharmaceutically acceptable salt thereof and the KRAS G12C inhibitor are administered and zongertinib or the pharmaceutically acceptable salt thereof is administered after the KRAS G12C inhibitor.

In another aspect, the KRAS G12C inhibitor is administered immediately before zongertinib or the pharmaceutically acceptable salt thereof. In this context, “immediately before” means, e.g. 30 minutes, 1 hour, 2 hours, 3 hours or 4 hours before.

It has been shown herein that there is a synergistic effect, defined as a greater than additive benefit, provided by co-administration of zongertinib or a pharmaceutically acceptable salt thereof and a KRAS G12C inhibitor as defined herein in the treatment of cancer. It is believed that, by acting simultaneously, superior inhibition of cancer can be achieved leading to improved cancer treatment outcomes, at least for cancers normally susceptible to treatment by anti-HER2 therapies.

In another aspect, the combination produces a synergistic therapeutic effect as compared to the sole administration of zongertinib or the pharmaceutically acceptable salt thereof or of the sole administration of the KRAS G12C inhibitor.

Although any combination of doses may be used, typically doses of the KRAS G12C inhibitor and zongertinib or the pharmaceutically acceptable salt thereof, that provide a synergistic effect, or greater than additive benefit, are used. For example, doses of the KRAS G12C inhibitor may be selected to synergistically lower overall toxicity when administered with zongertinib or the pharmaceutically acceptable salt thereof, while maintaining substantially the same overall treatment effect on cancerous cells as observed when the anti-HER2 antibody or anti-HER2 antibody-drug conjugate is administered alone, and vice-versa. In another example, doses of the KRAS G12C inhibitor when administered synergistically with zongertinib or the pharmaceutically acceptable salt thereof, may be selected to produce substantially the same overall toxicity while synergistically increasing the treatment effect on cancerous cells as observed when the KRAS G12C inhibitor is administered alone; and vice-versa. Due to this synergistic behavior, the KRAS G12C inhibitor may be advantageously administered at doses lower than currently approved doses by co-administration with zongertinib or the pharmaceutically acceptable salt thereof according to the invention without substantially reducing the efficacy of the cancer treatment. This has the benefit of reducing toxicity of the KRAS G12C inhibitor. In addition, the toxicity of zongertinib or the pharmaceutically acceptable salt thereof being co-administered may be less due to either a lower required dose or improved toxicological properties; this has the effect of further lowering overall toxicity of the combination without compromising the overall treatment effect.

The use of lower doses of the KRAS G12C inhibitor and/or zongertinib or the pharmaceutically acceptable salt thereof are, for example, at most about 80%, about 50%, about 40%, about 30%, about 20%, about 10%, about 5%, about 3%, about 2%, about 1%, about 0.75%, about 0.5%, about 0.25%, or about 0.1%, etc. of the dose of the KRAS G12C inhibitor and/or zongertinib or the pharmaceutically acceptable salt thereof used alone. At these doses, a synergistic effect in the treatment of cancerous cells may be observed.

### Additional Intervention

The combination of zongertinib, or a pharmaceutically acceptable salt thereof, and KRAS G12C inhibitor as described herein may be administered in combination with additional intervention selected from the group consisting of radiation, surgery, an additional therapeutic agent such as chemotherapy and a combination thereof.

In one aspect, the present invention relates to a use of the combination of zongertinib, or a pharmaceutically acceptable salt thereof, and KRAS G12C inhibitor as described herein in combination with a cytostatic and/or cytotoxic active substance and/or in combination with radiotherapy and/or in combination with surgery and/or in combination with chemotherapy and/or in combination with immunotherapy in the treatment and/or prevention of cancer.

For the treatment of diseases of oncological nature, anticancer agents may be combined with radiotherapy, e.g. irradiation treatment, and/or surgery. The combination of zongertinib, or a pharmaceutically acceptable salt thereof, and KRAS G12C inhibitor, as well as the relative compounds for use, methods of treatment, uses, pharmaceutical compositions and kits as described herein, can be used in combination with radiotherapy. For example, a cancer patient may receive radiotherapy before and/or after or simultaneously with receiving therapy with the combination of zongertinib, or a pharmaceutically acceptable salt thereof, and KRAS G12C inhibitor, as well as with the relative compounds for use, methods of treatment, uses, pharmaceutical compositions and kits as described herein.

In embodiments, the combination of zongertinib, or a pharmaceutically acceptable salt thereof, and KRAS G12C inhibitor, as well as the relative compounds for use, methods of treatment, uses, pharmaceutical compositions and kits as described herein, can be used as adjuvant therapy in combination with a surgical procedure. The combination of zongertinib, or a pharmaceutically acceptable salt thereof, and KRAS G12C inhibitor, the relative compounds for use, methods of treatment, uses, pharmaceutical compositions and kits described herein may be administered for the purpose of diminishing the size of a tumor before surgical procedure (referred to as pre-operative adjuvant chemotherapy or neoadjuvant therapy), or may be administered after a surgical procedure for the purpose.

In embodiments, the combination of zongertinib, or a pharmaceutically acceptable salt thereof, and KRAS G12C inhibitor, the relative compounds for use, methods of treatment, uses, pharmaceutical compositions and kits as described herein may be administered in combination with a cytostatic and/or cytotoxic active substance and/or in combination with immunotherapy.

In embodiments, the combination of zongertinib, or a pharmaceutically acceptable salt thereof, and KRAS G12C inhibitor, as well as the relative compounds for use, methods of treatment, uses, pharmaceutical compositions and kits as described herein may be used in combination with one or several other pharmacologically active substances such as state-of-the-art or standard-of-care compounds, such as e.g. cell proliferation inhibitors, anti-angiogenic substances, steroids or immune modulators/checkpoint inhibitors, and the like.

Pharmacologically active substances which may be administered in combination with the combination of zongertinib, or a pharmaceutically acceptable salt thereof, and KRAS G12C inhibitor, as well as the relative compounds for use, methods of treatment, uses, pharmaceutical compositions and kits as described herein, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib, bevacizumab, pertuzumab and trastuzumab); antimetabolites (e.g. antifolates such as methotrexate, raltitrexed, pemetrexed, pyrimidine analogues such as 5fluorouracil (5fluorineU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kat inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, KRAS inhibitors (e.g. KRAS G12C inhibitors), signalling pathway inhibitors (e.g. SOS1 inhibitors), FLT3 inhibitors, BRD4 inhibitors, IGF-1R inhibitors, TRAILR2 agonists, Bel-xL inhibitors, Bel-2 inhibitors, Bel-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), T-cell engagers (e.g. bi-specific T-cell engagers (BiTEs®) like e.g. CD3×BCMA, CD3×CD33, CD3×CD19), PSMA×CD3), tumor vaccines and chemotherapeutic agents, chemoprotective and supportive agents such as eribulin, amifostin, anagrelid, clodronat, filgrastin, interferon, interferon alpha, leucovorin, procarbazine, levamisole, mesna, mitotane, pamidronate and porfimer.

### Cancer

In embodiments, the cancer is one of the following, without being restricted thereto:

Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands); cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), appendix, genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas;
Gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; nonfluorineunctional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
peripheral nervous system cancer;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (BchlorineL), chronic T-cell lymphocytic leukemia (TchlorineL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP).

All cancers/tumors/carcinomas mentioned above which are characterized by their specific location/origin in the body are meant to include both the primary tumors and the metastatic tumors derived therefrom. Preferably, the cancer as defined herein (including in any embodiment referring to e.g. cancer types) is metastatic, advanced, and/or unresectable.

All cancers/tumors/carcinomas mentioned above may be further differentiated by their histopathological classification:

Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma; Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

In some embodiments, the cancer is a solid tumor. In some embodiments, the cancer is manifested by at least one solid tumor.

In some embodiments, the cancer is selected from the group consisting of brain cancer, breast cancer, endocrine cancer, gastrointestinal cancer, gynecologic cancer, head and neck tumor, lung cancer, nervous system cancer, and skin cancer.

Preferably, said brain cancer is a glioblastoma or a glioma.

Preferably, said breast cancer is lobular breast cancer. In addition or in alternative, said breast cancer is preferably metastatic.

Preferably, said endocrine cancer is nerve sheath tumor, more preferably HER2 mutant nerve sheath tumor.

Preferably, said gastrointestinal cancer is selected from the group consisting of anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer and small bowel cancer. In addition or in alternative, said gastrointestinal cancer may be a gastrointestinal neuroendocrine tumor, preferably HER2 mutant.

Still preferably, said gastrointestinal cancer is selected from the group consisting of gastric adenocarcinoma, gastroesophageal junction adenocarcinoma and esophageal adenocarcinoma, in particular metastatic gastric adenocarcinoma, metastatic gastroesophageal junction adenocarcinoma and metastatic esophageal adenocarcinoma.

Preferably, said gynecologic cancer is selected from the group consisting of cervical cancer, uterine cancer, endometrial cancer and ovarian cancer.

As used herein, “head and neck tumor” preferably refers to a head and neck cancer.

Preferably, said head and neck tumor is a salivary gland cancer or tumor.

Preferably, said lung cancer is non-small cell lung cancer (NSCLC).

Preferably, said nervous system cancer is peripheral nervous system cancer, more preferably HER2 amplified peripheral nervous system cancer.

Preferably, said skin cancer is not a melanoma, i.e. non-melanoma skin cancer.

In some embodiments, the cancer is selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In some embodiments, the cancer is HER2 overexpressed, HER2 amplified and/or HER2 mutant (in particular HER2 exon 20 mutant) cancer selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In another aspect, the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In some embodiments, the cancer is HER2 overexpressed, HER2 amplified and/or HER2 mutant (in particular HER2 exon 20 mutant) cancer selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In further embodiments, the cancer is selected from cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer). In still further embodiments, the cancer is NSCLC. In still further embodiments, the cancer is HER2 exon 20 mutant NSCLC.

In another aspect, the cancer is selected from the group consisting of breast cancer, esophageal cancer, gastric cancer, gastroesophageal junction cancer, lung cancer and ovarian cancer. In another aspect, the cancer is selected from the group consisting of breast cancer, esophageal cancer, gastric cancer, gastroesophageal junction cancer, and lung cancer. Preferably, said lung cancer is non-small cell lung cancer (NSCLC).

In another aspect, said cancer is advanced, unresectable and/or metastatic.

In a further preferred embodiment, said cancer is advanced and metastatic. In a further preferred embodiment, when said cancer is metastatic, the metastases are located in the lung, lymph node, bone or liver. In a further preferred embodiment, the cancer is advanced cancer including metastases and the metastases are located in the lung, lymph node, bone or liver.

In a preferred embodiment the cancer is unresectable advanced cancer comprising solid tumors and solid metastases and the metastases are located in the lung, liver, lymph node-tissue or bone.

In another aspect, said cancer is an adenocarcinoma.

In another aspect, the cancer or tumor comprises a HER2 aberration. This means that the cells of the cancer or tumor harbor an aberration of HER2. As used herein, the expressions “HER2 aberration”, “aberration of HER2” and grammatical variants thereof have the meaning commonly attributed to them in the art and include any variation or alteration in the HER2 protein or its encoding gene, such as: overexpression of the HER2 protein, amplification of the HER2-encoding gene, mutations in the HER2-encoding gene and/or in the HER2 protein (in particular non-synonymous mutations, somatic mutations, mutations in specific regions, e.g. in the tyrosine kinase domain, in exon 20, etc.) as well as gene rearrangements of HER2 and/or NRG1. When the cancer comprises a HER2 aberration, it can be referred to as HER2 aberrant. When the cancer comprises an overexpression of the HER2 protein, it can be referred to as HER2 overexpressed. When the cancer comprises an amplification of the HER2-encoding gene, it can be referred to as HER2 amplified. When the cancer comprises a mutation in the HER2-encoding gene and/or in the HER2 protein, it can be referred to as HER2 mutant.

In another aspect, the cancer is HER2 overexpressed, HER2 amplified and/or HER2 mutant.

In another aspect, the cancer comprises a mutation in the tyrosine kinase domain of HER2.

In another aspect, the cancer is HER2 overexpressed and/or HER2 amplified.

In another aspect, the cancer comprises a gene rearrangement of HER2 and/or NRG1.

The presence or absence of HER2 alterations, including overexpression, amplification or mutations, can be determined using methods known in the art.

“HER2 overexpressed” as used herein refers to a cancer comprising cells that express HER2 at levels detectable by immunohistochemistry (e.g. IHC 2+ and IHC 3+) and/or methods assaying ERBB2 messenger RNA.

“HER2 amplified” as used herein refers to a cancer comprising cells exhibiting more than 2, in particular more than 3, 4, 5, 6, 7, 8, 9 or 10, preferably more than 6, copies of the HER2 gene ERBB2.

HER2 expression, gene copy number and amplification can be measured, for example, by determining nucleic acid sequencing (e.g., sequencing of genomic DNA or cDNA), measuring mRNA expression, measuring protein abundance, or a combination thereof. HER2 testing methods include immunohistochemistry (IHC), in situ hybridization—including fluorescence in situ hybridization (FISH), chromogenic in situ hybridization (CISH), ELISAs, and RNA quantification using techniques such as Reverse Transcription-Polymerase Chain Reaction (RT-PCR), microarray analysis and Next Generation Sequencing (NGS). HER2 expression in or on the cancer sample cells can be compared to a reference cell. The reference cell can be a non-cancer cell obtained from the same subject as the sample cell. The reference cell can be a non-cancer cell obtained from a different subject or a population of subjects.

When the cancer is HER2 overexpressed and/or HER2 amplified in or on a cell, the cancer can be referred to as being “HER2 positive”. The level of HER2 amplification or overexpression in HER2 positive cancers is commonly expressed as a score ranging from 0 to 3 (i.e., HER2 0, HER2 1+, HER2 2+, or HER2 3+), with higher scores corresponding to greater degrees of expression.

In an aspect, the cancer is HER2 positive.

Preferably, “HER2 overexpressed”, “HER2 amplified” and “HER2 positive” mean that the cancer comprises cells having an immunohistochemistry score of 2+ or 3+.

Preferably, “HER2 overexpressed”, “HER2 amplified” and “HER2 positive” mean that the cancer comprises cells having HER2 amplification defined by in situ hybridization.

In some embodiments, the HER2 status of the cancer, specifically of a sample cell within the cancer, is determined. The determination can be made before the combination treatment begins, during treatment, or after treatment has been completed. In some instances, determination of the HER2 status results in a decision to change therapy (e.g., switching to a different KRAS G12C inhibitor or switching from another treatment method to a method of the present invention).

The sample cell can be obtained as a biopsy specimen, by surgical resection, or as a fine needle aspirate (FNA).

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2.

“HER2 mutant” as used herein refers to a cancer harbouring at least one mutation, i.e. an alteration in the nucleic acid sequence of the HER2-encoding gene and/or an alteration in the amino acid sequence of the HER2 protein, including but not limited to those listed below. Mutations can be found with any method known to the skilled person, such as molecular diagnostic methods including but not limited to Polymerase Chain Reaction (PCR), Single Strand Conformational Polymorphism (SSCP), Denaturing Gradient Gel Electrophoresis (DGGE), Heteroduplex analysis, Restriction fragment length polymorphism (RFLP), Next Generation Sequencing (NGS) and Whole Exome Sequencing.

In embodiments of said HER2 mutant cancer, the mutation is a non-synonymous mutation. As used herein, the term “non-synonymous” has the meaning commonly attributed to it in the art and in particular refers to a mutation in the nucleic acid sequence of the HER2-encoding gene that alters the amino acid sequence of the HER2 protein.

In embodiments of said HER2 mutant cancer, the mutation is a somatic mutation. As used herein, the term “somatic” has the meaning commonly attributed to it in the art and in particular refers to a mutation in the nucleic acid sequence of the HER2-encoding gene occurring in a cell other than a gamete, a germ cell or a gametocyte.

In embodiments of said HER2 mutant cancer, the mutation is a non-synonymous somatic mutation.

In embodiments of said HER2 mutant cancer, the mutation is a non-synonymous somatic mutation in the tyrosine kinase domain of HER2.

In embodiments of said HER2 mutant cancer, the mutation is in the tyrosine kinase domain of HER2, in particular in the exon 20 of HER2. In the latter case, the cancer can be referred to as HER2 exon 20 mutant.

As used herein, a cancer comprising a mutation in the tyrosine kinase domain of HER2 is a cancer where the cancer or tumor cells harbour at least one mutation in the tyrosine kinase domain of HER2, which ranges from amino acids 694 to 883 and/or exons 18 to 21.

“Cancer with HER2 exon 20 mutation” or “HER2 exon 20 mutant cancer” as used herein refers to a cancer where the cancer or tumor cells harbour at least one HER2 exon 20 mutation including but not limited to the mutations listed below.

ERBB2 (HER2) exon 20 encodes for a part of the kinase domain and ranges from amino acids 769 to 835. Every mutation, insertion, duplication or deletion within this region is defined as an exon 20 mutation including the following mutations: p.A772_G773insMMAY; p.Y772_A775_dup (YVMA); p.A775_G776insYVMA; p.Y772insYVMA; p.M774delinsWLV; p.A775_G776insSVMA; p.A775_G776insVVMA; p.A775_G776insYVMS; p.A775_G776insC; p.A776_delinsVC; p.A776_delinsLC; p.A776_delinsVV; p.A776_delinsAVGC; p.A776_delinsIC; p.A776_V777delinsCVC; p.V777_insE; p.V777_G778insV; p.V777_G778insC; p.V777_G778insCG; p.V777_S779dup; p.V777L; p.V777M; p.G778_P780dup (GSP); p.G778_S779insCPG; p.G778_S779insG; p.G776_delinsVC; p.G776_V777delinsAVGCV; p.G776delinsLC; p.G776_V777delinsAVCV; p.G776delinsVV; p.G776_V777insL; p.G776_V777insVGC; p.G776C; p.G776A; p.G776L; p.G776V; p.P780_Y781insGSP (“p.” is referring to the HER2 protein). In addition HER2 mutations exist outside of exon 20 including the following mutations: p.S310A; p.S310F; p.S310Y; p.R678Q; p.G727A; p.T733I; p.L755S; p.L755A; p.L755F; p.L755P; p.L755S; p.V842I; p.D769Y; p.D769H; p.R103Q; p.G1056S; p.I767M; p.L869R; p.L869R; p.T733I; p.T862A; p.V697L; p.R929W; p.D277H; p.D277Y; p.G660D (“p.” is referring to the HER2 protein).

Of these, examples of tyrosine kinase mutations include: p.G727A; p.T733I; p.L755S; p.L755A; p.L755F; p.L755P; p.L755S; p.V842I; p.D769Y; p.D769H; p.I767M; p.L869R; p.L869R; p.T733I; p.T862A; p.V697L.

In an aspect, the cancer is HER2 positive breast cancer, in particular advanced HER2 positive breast cancer or HER2 positive metastatic breast cancer, preferably advanced HER2 positive metastatic breast cancer. Preferably, in this embodiment, the combination as described herein is administered as first line of therapy. Still preferably, in this embodiment, the combination as described herein is administered as second, third or further line of therapy.

In an aspect, the cancer is HER2 positive esophageal cancer, HER2 positive gastric cancer, or HER2 positive gastroesophageal junction cancer, in particular HER2 positive esophageal adenocarcinoma, HER2 positive gastric adenocarcinoma, or HER2 positive gastroesophageal junction adenocarcinoma, preferably metastatic HER2 positive esophageal adenocarcinoma, metastatic HER2 positive gastric adenocarcinoma, or metastatic HER2 positive gastroesophageal junction adenocarcinoma. Preferably, in this embodiment, the combination as described herein is administered as first line of therapy. Still preferably, in this embodiment, the combination as described herein is administered as second or further line of therapy.

In an embodiment, the cancer is advanced, unresectable or metastatic NSCLC harbouring a HER2 mutation, wherein said HER2 mutation is in the tyrosine kinase domain. Preferably, in this embodiment, the combination as described herein is administered as first line of therapy. Still preferably, in this embodiment, the combination as described herein is administered as second or further line of therapy.

In an aspect, the cancer is HER2 mutant lung cancer, in particular HER2 exon 20 mutant lung cancer or HER2 mutant NSCLC, preferably HER2 exon 20 mutant NSCLC.

In another aspect, the cancer is KRAS G12C mutant.

In another aspect, the cancer is resistant to treatment with the KRAS G12C inhibitor. In another aspect, the cancer is resistant to single-agent treatment with the KRAS G12C inhibitor as defined herein. In these aspects, the resistance may be advantageously overcome by treatment with a combination of zongertinib or a pharmaceutically acceptable salt thereof and the KRAS G12C inhibitor.

Cancer showing/developing “resistance” or being/becoming “resistant” to a therapy as used herein includes a cancer which is not responsive and/or exhibits reduced ability of producing a significant response, e.g., partial response and/or complete response, to treatment with the KRAS G12C inhibitor.

Resistance may be de novo (primary) resistance or acquired resistance which arises in the course of a treatment method. The term “acquired resistance” as used herein indicates that the cancer becomes resistant and/or substantially less responsive to the effects of the KRAS G12C inhibitor after being exposed to it for a certain period of time.

Cancer may develop resistance to treatment, in particular single-agent treatment, with an inhibitor of KRAS G12C. A cancer which initially responded to an KRAS G12C inhibitor can relapse and become resistant to the KRAS G12C inhibitor when the KRAS G12C inhibitor is no longer effective in treating a subject with the cancer, for example despite the administration of increased dosages of the KRAS G12C inhibitor.

The cancer may be recurrent, relapsed, resistant or refractory to one or more KRAS G12C inhibitors. Thus, the patients may have received previous anti-cancer therapies with one or more KRAS G12C inhibitors, which have not completely cured the disease. In embodiments, the cancer is resistant to treatment with sotorasib (AMG 510) and/or adagrasib (MRTX 849). In embodiments, the cancer has become resistant to treatment with sotorasib (AMG 510) and/or adagrasib (MRTX 849) after earlier treatment with sotorasib (AMG 510) and/or adagrasib (MRTX 849).

Cancer with relapse and/or with resistance to KRAS G12C inhibitor may be particularly amenable for combined treatment with HER2 tyrosine kinase inhibitor zongertinib, or a pharmaceutically acceptable salt thereof, such as for second or further line treatment cycles (optionally in further combination with one or more other anti-cancer agents), or as add-on combination or as replacement treatment. A combination of zongertinib or a pharmaceutically acceptable salt thereof, and a KRAS G12C inhibitor may be effective at treating subjects whose cancer has relapsed, or whose cancer has become drug resistant to KRAS G12C inhibitors, or whose cancer has failed one, two or more lines of mono- or combination therapy with one or more KRAS G12C inhibitors.

### Pharmaceutical Composition and Kit

It is provided a pharmaceutical composition comprising zongertinib or a pharmaceutically acceptable salt thereof, a KRAS G12C inhibitor and a pharmaceutically acceptable excipient. Preferably, said pharmaceutical composition is for use in the treatment and/or prevention of cancer. Also provided herein is a method of treating and/or preventing cancer, the method comprising administering to a patient in need thereof said pharmaceutical composition, wherein optionally the pharmaceutical composition comprises a therapeutically effective amount of zongertinib or the pharmaceutically acceptable salt thereof and of the KRAS G12C inhibitor. Also provided herein is a use of said pharmaceutical composition as defined herein for the manufacture of a medicament for the treatment and/or prevention of cancer. In these aspects, zongertinib, the pharmaceutically acceptable salt thereof, the cancer and the KRAS G12C inhibitor can each independently or together be as described in any of the aspects or embodiments disclosed herein. In addition or in alternative, in these aspects, zongertinib or the pharmaceutically acceptable salt thereof and/or the KRAS G12C inhibitor can be administered according to the dosing schedules defined herein.

The term “pharmaceutically acceptable excipient” refers to a non-toxic component that does not destroy the pharmacological activity of the compound with which it is formulated. Pharmaceutically acceptable excipients that may be used in the compositions include fillers, disintegrants, glidants, lubricants, and coating agents. The compositions may comprise further pharmaceutically acceptable excipients selected from buffers, dispersion agents, surfactants, wetting agents, emulsifiers, suspending agents, preservatives, antioxidants, opaquing agents, processing aids, colorants, sweeteners, perfuming agents, flavoring agents, diluents and other known additives usable in the manufacturing of a pharmaceutical product. Pharmaceutical compositions as referred to herein may contain conventional non-toxic pharmaceutically acceptable excipients.

Also provided herein is a pharmaceutical composition comprising zongertinib or a pharmaceutically acceptable salt thereof and a pharmaceutically acceptable excipient, for use in the treatment and/or prevention of cancer, wherein the pharmaceutical composition is administered in combination with a KRAS G12C inhibitor.

The terms “first” and “second” with respect to pharmaceutical compositions, as used herein, are solely intended to indicate that these compositions are two different compositions. Thus, these terms shall not be understood to refer to the order or sequence of administration.

In an embodiment of the kit as defined herein, the first and second pharmaceutical compositions are packaged together in a single container.

In an embodiment, the kit as defined herein additionally comprises a package insert. Preferably, the package insert comprises instructions. Still preferably, the instructions provide guidance on simultaneous, concurrent, sequential, successive, alternate or separate administration of zongertinib, its pharmaceutically acceptable salt or pharmaceutical composition and the KRAS G12C inhibitor.

In an embodiment, the kit is for use as a medicament.

In an embodiment, the kit is for use in the treatment and/or prevention of cancer.

In an embodiment, the kit further comprises instructions for administering the first and second pharmaceutical compositions concurrently.

In an embodiment, the kit further comprises instructions specifying a type of cancer to treat and the cancer is as defined herein, in particular the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In an embodiment, the kit further comprises instructions specifying a type of cancer to treat and the cancer is as defined herein, in particular the cancer is HER2 overexpressed, HER2 amplified and/or HER2 mutant cancer and/or the cancer is KRAS G12C mutant and/or the cancer is resistant to treatment with the KRAS G12C inhibitor.

In an embodiment, the kit further comprises instructions specifying at least one dosage that produces a synergistic therapeutic effect in a patient as compared to the sole administration of zongertinib or the pharmaceutically acceptable salt thereof, or to the sole administration of an individual combination partner.

Also provided herein is a method of treating and/or preventing cancer, the method comprising administering to a patient in need thereof a kit comprising:
(i) a first pharmaceutical composition comprising zongertinib or a pharmaceutically acceptable salt thereof and a pharmaceutically acceptable excipient; and
(ii) a second pharmaceutical composition comprising a KRAS G12C inhibitor and a pharmaceutically acceptable excipient.

In an embodiment, the first pharmaceutical composition comprises a therapeutically effective amount of zongertinib or the pharmaceutically acceptable salt thereof and the second pharmaceutical composition comprises a therapeutically effective amount of the KRAS G12C inhibitor.

Also provided herein is a use of the kit as defined herein for the manufacture of a medicament for the treatment and/or prevention of cancer.

Features and advantages of the present invention will become apparent from the following detailed examples, which illustrate the principles of the invention by way of example without restricting its scope:

### Abbreviations

BID Twice daily (latin: bis in die)
DMSO Dimethyl sulfoxide
DX Deruxtecan
EGFR Epidermal growth factor receptor
ERBB erb-b receptor tyrosine kinase
ERBB2 erb-b2 receptor tyrosine kinase 2
FCS Fetal calf serum
HER2 Human epidermal growth factor receptor 2
HER3 Human epidermal growth factor receptor 3
HER4 Human epidermal growth factor receptor 4
MAP Mitogen-activated protein
NSCLC Non-small cell lung cancer
PBS phosphate-buffered saline
PI3K Phosphoinositide 3-kinase
p.o. oral
QD once daily (latin: quaque die)
ROA route of administration
RPMI 1640 Roswell Park Memorial Institute (RPMI) 1640 formula medium
RTK Receptor tyrosine kinase
T-DM1 trastuzumab-DM1, ado-trastuzumab emtansine
TGI Tumor growth inhibition, calculated to the formula:

TGI=100×{1−[(treatedfinal day−treatedday1)/(controlfinal day−controlday1)]}

### Example 1) Combination of Zongertinib with Adagrasib or Sotorasib in Xenografts Derived from NCI-H2122 (KRAS^{G12C}-Mutant Non-Small Cell Lung Cancer)

In this Example, the anti-tumor activity of zongertinib in combination with the KRAS^{G12C} inhibitors adagrasib or sotorasib is evaluated in a subcutaneous xenograft mouse model derived from the KRAS^{G12C}-mutant human non-small cell lung cancer (NSCLC) cell line NCI-H2122 in NMRI-Foxn1^{nu} mice. The NCI-H2122 xenograft model expresses KRAS^{G12C} and is dependent on KRAS^{G12C} but not HER2.

### 1.1 Materials & Methods

### 1.1.1 Study Design

The study is performed in human non-small cell lung cancer (NSCLC) cell line NCI-H2122 grown as subcutaneous xenografts in NMRI-Foxn1^{nu} mice. For the combination with adagrasib, the study is designed as shown in Table 1. For the combination with sotorasib, the study is designed as shown in Table 2.

TABLE 1
Treatment groups for studying the combination of zongertinib
with adagrasib in xenografts derived from NCI-H2122

| No. of | | Dose | | | |
|---|---|---|---|---|---|
| mice | Treatment | [mg/kg] | | Schedule | Route |
| 10 | Control | — | | qd | p.o. |
| 8 | Zongertinib | 20 | mg/kg | | |
| 8 | Adagrasib | 100 | mg/kg | | |

| | | |
|---|---|---|
| 8 | Zongertinib + | 20 mg/kg |
| | Adagrasib | 100 mg/kg |

TABLE 2
Treatment groups for studying the combination of zongertinib
with sotorasib in xenografts derived from NCI-H2122

| No. of | | Dose | | | |
|---|---|---|---|---|---|
| mice | Treatment | [mg/kg] | | Schedule | Route |
| 10 | Control | — | | qd | p.o. |
| 8 | Zongertinib | 20 | mg/kg | | |
| 8 | Sotorasib | 100 | mg/kg | | |

| | | |
|---|---|---|
| 8 | Zongertinib + | 20 mg/kg |
| | Sotorasib | 100 mg/kg |

### 1.1.2 Cells

NCI-H2122 is a non-small cell lung cancer cell line that can be obtained from the American Type 10 Culture Collection (ATCC #CRL-5985) that expresses a KRAS^{G12C} mutation. Cells are cultured in T175 tissue culture flasks at 37° C. and 5% CO₂. The medium used is RPMI 1640 (ATCC-Formulation provided by Gibco #A10491) supplemented with 10% fetal calf serum (FCS) (Thermo Scientific #SH 30071.03). Cultures are split twice weekly with a ratio of 1:2-1:4. TrypLE Express (Gibco 12604-013) is used to split cells.

### 1.1.3 Mice

Mice are 6- to 10-week-old female NMRI-Foxn1^{nu} purchased from Taconic, Denmark. Upon arrival at the animal facility, mice are allowed to adjust to conditions at least for 5 days before they are used for the experiment. They are housed in Macrolon® type III cages in groups of 8 to 10 under pathogen-free, controlled and standardized conditions at 21.5±1.5° C. temperature, 55±10% humidity and 12-h light-dark cycle. Standardized diet (PROVIMI KLIBA) and autoclaved tap water are provided ad libitum. Subcutaneous microchips implanted under isoflurane anesthesia are used to identify each mouse. Cage cards showing the study number, the animal identification number, the compound, the dose level, the administration route as well as the schedule remain with the animals throughout the study.

### 1.1.4 Establishment of Tumors and Randomization

To establish subcutaneous tumors, NCI-H2122 cells are harvested by centrifugation, washed and resuspended in PBS+5% FCS at 5×10⁷ cells/ml. 100 μl cell suspension containing 5×10⁶ cells is injected subcutaneously into the right flank of the mice (1 site per mouse). Mice are randomly distributed between the treatment and the vehicle control group (15 days after cell injection) when tumors are well established and have reached volumes of 44 to 304 mm³ for the combination with adagrasib or 109 to 252 mm³ for the combination with sotorasib.

### 1.1.5 Administration of Test Compounds

To prepare stock solutions, zongertinib and sotorasib are both suspended in Natrosol (0.5%) and adagrasib in Natrosol (0.5%) added with 1% DMSO. Compounds are administered intragastrally by gavage needle with a volume of 10 ml/kg body weight.

Solutions are used for a maximum of 7 days after preparation. For combinations, stock solutions are mixed freshly every day.

Mice in the control group are treated with vehicle, i.e. 0.5% Natrosol for the sotorasib experiment and 0.5% Natrosol added with 1% DMSO for the adagrasib experiment, administered once daily for the duration of the treatment intragastrally by gavage needle with a volume of 10 ml/kg body weight.

### 1.1.6 Monitoring Tumor Growth and Side Effects

Tumor volumes are measured three times a week with a caliper. The volume of each tumor [in mm³] is calculated according to the formula:

$$tumor volume = length \times {diameter}^{2} \times \frac{π}{6}$$

Median tumor volumes for each treatment group are plotted over time in the figures. The percent change from baseline is calculated as:

$$Change from baseline ( \% ) = ( tumor {volume}_{last day} / tumor {volume}_{1 st day} - 1 ) \times 100.$$

To monitor side effects of treatment, mice are inspected daily for abnormalities and body weight is determined three times a week. Control animals are sacrificed when the median tumor volume of the group reaches a size of approximately 950 mm³ for the combination with adagrasib or 1167 mm³ for the combination with sotorasib. In addition, animals with tumor sizes exceeding 1500 mm³ in diameter or with ulcerating tumors are euthanized for ethical reasons.

### 1.1.7 Statistical Analysis

The statistical evaluation of the tumor volume and the body weight is conducted on day 23 (last day of the controls) for the combination with adagrasib or on day 15 (last day of the controls) for the combination with sotorasib.

In the results reported below with adagrasib, animal no. 10 (controls) was excluded from the statistical evaluation as it had to be euthanized due to tumor necrosis (day 18). Animal no. 18 (20 mg/kg zongertinib qd) had to be euthanized due to tumor size (day 14) and the last tumor volume was carried forward to day 23.

Absolute values are used for tumor volumes. Due to the observed variation, nonparametric methods are applied.

The number of animals, the median, the minimum and the maximum of the tumor volume is calculated for each group for descriptive considerations.

For a quick overview of possible treatment effects, the median of the tumor volume of each treatment group T is referred to the median of the control C as tumor growth inhibition (TGI) defined as:

$$TGI = 100 \times \{ 1 - [ \frac{( {treated}_{final day} - {treated}_{day 1} )}{( {control}_{final day} - {control}_{day 1}} ] \}$$

The percentage change referring to the initial weight of the first treatment day is used as target variable for the statistical analysis for the body weight.

Due to the observed variation, nonparametric methods are applied.

For the statistical evaluation two subtopics are of interest:
Monotherapy effects—comparison of the therapies versus the control group
Effect of the combination—comparison of combination versus monotherapies

One-sided non-parametric Mann-Whitney-Wilcoxon U-tests str applied to compare all treatment groups with the control, as well as the combination therapy group with the corresponding monotherapy groups, looking for:
a decrease in the tumor volume (inhibition of tumor growth, efficacy parameter)
a decrease in the body weight change (body weight loss, tolerability parameter)

Within each subtopic, the p values of the efficacy parameters are adjusted for multiple comparisons according to Bonferroni-Holm. The p values of the tolerability parameter remain unadjusted in order not to overlook a possible adverse effect.

The level of significance is fixed at α=5%. An adjusted p value of less than 0.05 is considered to show a statistically significant difference between the groups and differences are seen as indicative whenever 0.05≤p value<0.10.

As target variable, the time until the tumor reaches a critical volume of 500 mm³ is included. If an animal dies or is sacrificed without reaching the critical tumor volume, the time point is set to the last measurement time point and included as a censored observation.

For descriptive considerations the number of animals, the median, the minimum and the maximum of the time to critical tumor volume is calculated.

The median survival time is derived from the Kaplan Meier survival curves per group as the time point with a survival probability (probability of reaching the critical tumor volume) of 0.5. For the treatment groups the median survival time is referred to the corresponding time of the control group as an estimate for the prolongation of the time to critical tumor volume by the therapy.

For the comparisons of the several groups a logrank test is applied.

The statistical evaluation is prepared using the software Graph Pad Prism.

### 1.2 Results

1.2.1 Combination with Adagrasib

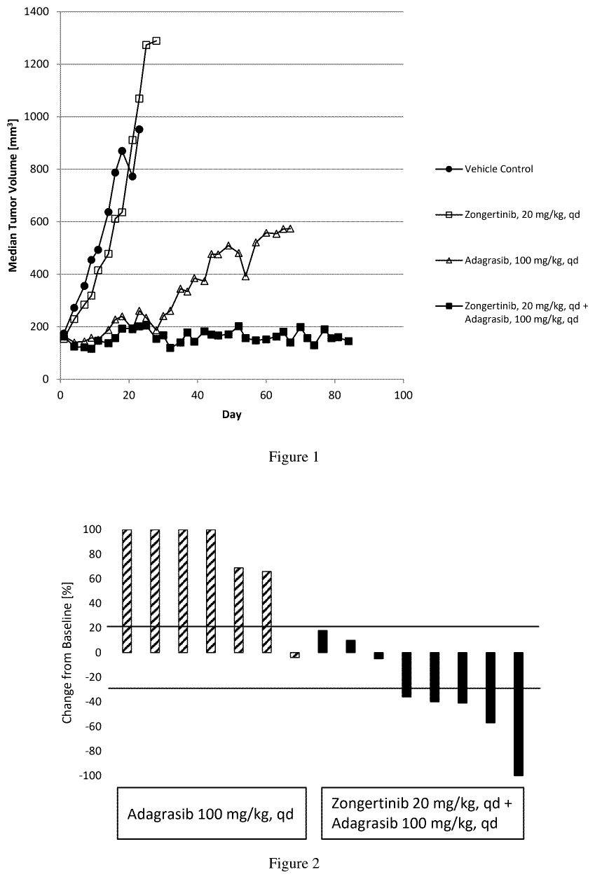

Results are summarised in Table 3 and in FIGS. 1 and 2.

TABLE 3
TGI at day 23 for each treatment group for studying the combination
of zongertinib with adagrasib in xenografts derived from NCI-H2122

| | | | | | Time to | | |
|---|---|---|---|---|---|---|---|
| | | | p value | p value | reach | p value | p value |
| | Dose, | TGI | vs. | vs. | 500 mm³ | vs. | vs. |
| Compound | schedule | [%] | control | * | [Days] | control | * |
| Control | — qd | | | | 12.5 | | |
| Zongertinib | 20 mg/kg, qd | −18 | 0.4813 | 0.0004 | 15.0 | 0.9649 | <0.0001 |
| Adagrasib | 100 mg/kg, qd | 88 | 0.0078 | 0.2209 | 53.0 | 0.0114 | 0.0022 |
| Zongertinib + | 20 mg/kg, qd | 95 | 0.0015 | * | >84.0 | 0.0002 | * |
| Adagrasib | 100 mg/kg, qd | | | | | | |

* indicates reference group for comparison
bold adjusted value < 0.05

In the subcutaneous KRAS^{G12C}-mutant human non-small cell lung carcinoma model NCI-H2122, zongertinib showed no significant anti-tumor activity when administered orally at 20 mg/kg qd. Adagrasib administered orally at 100 mg/kg qd showed a significant anti-tumor activity. Combination of zongertinib with adagrasib showed significantly higher efficacy on tumor growth compared to both monotherapies (FIG. 1) and induced strong tumor regressions in 5 out of 8 mice on day 23 (last day of the controls) (FIG. 2).

The time to reach tumor sizes of more than 500 mm³ was significantly longer in the combination group compared to both monotherapies.

All treatments were well tolerated.

1.2.2 Combination with Sotorasib

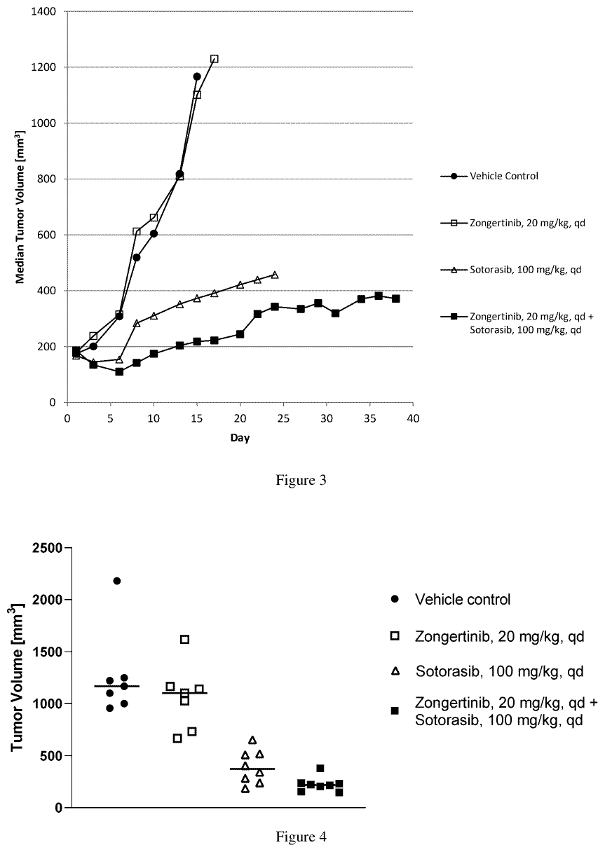

Results are summarised in Table 4 and in FIGS. 3 and 4.

TABLE 4
TGI at day 22 for each treatment group for studying the combination
of zongertinib with T-DXd in xenografts derived from NCI-N87

| | | | | | Time to | | |
|---|---|---|---|---|---|---|---|
| | | | p value | p value | reach | p value | p value |
| | Dose, | TGI | vs. | vs. | 500 mm³ | vs. | vs. |
| Compound | schedule | [%] | control | * | [Days] | control | * |
| Control | — qd | | | | 8.0 | | |
| Zongertinib | 20 mg/kg, qd | 7 | 0.1914 | 0.0004 | 8.0 | 0.3843 | <0.0001 |
| Sotorasib | 100 mg/kg, qd | 79 | 0.0006 | 0.0074 | 23.0 | <0.0001 | 0.0352 |
| Zongertinib + | 20 mg/kg, qd | 97 | 0.0006 | * | 38.0 | <0.0001 | * |
| Sotorasib | 100 mg/kg, qd | | | | | | |

* indicates reference group for comparison
bold adjusted value < 0.05

In the subcutaneous KRAS^{G12C}-mutant human non-small cell lung carcinoma model NCI-H2122, zongertinib showed no significant anti-tumor activity when administered orally at 20 mg/kg qd. Sotorasib administered orally at 100 mg/kg qd showed a significant anti-tumor activity. Combination of zongertinib with Sotorasib showed significantly higher efficacy on tumor growth compared to both monotherapies (FIGS. 3 and 4).

The time to reach tumor sizes of more than 500 mm³ was significantly longer in the combination group compared to both monotherapies.

All doses were well tolerated.

In summary, zongertinib monotherapy did not affect the growth of NCI-H2122 xenografts. Both sotorasib and adagrasib reduced but did not prevent tumor growth (FIGS. 1 and 3). Adding zongertinib to sotorasib further suppressed the tumor growth whereas adding zongertinib to adagrasib persistently abolished the tumor growth (FIGS. 1 and 3). It was demonstrated that zongertinib inhibits the growth of a KRAS^{G12C} dependent xenograft model synergistically with both sotorasib and adagrasib.

### Example 2) Combination of Zongertinib with Adagrasib in Xenografts Derived from Patients with KRAS^{G12C}-Mutant Colorectal Cancer

In this Example, the anti-tumor activity of zongertinib in combination with the KRAS^{G12C} inhibitor adagrasib is evaluated in a subcutaneous xenograft mouse model derived from KRAS^{G12C}-mutant colorectal cancer in NMRI-Foxn1^{nu} mice.

### 2.1 Materials & Methods

### 2.1.1 Study Design

The study is performed in 6-12 weeks old female Athymic Nude, Outbred Homozygous (Crl:NU(NCr)-Foxn1^{nu}) mice with a subcutaneous xenograft of the colorectal cancer cell line ST524. The study is designed as shown in Table 5.

TABLE 5
Treatment groups for studying the combination of zongertinib
with adagrasib in xenografts derived from ST524

| | No. of | | Dose | | Doses Administered | | Data |
|---|---|---|---|---|---|---|---|
| Group | mice | Treatment | (mg/kg) | ROA / Schedule | Total | T_{X} Day(s) | Endpoint |
| 1 | 10 | Zongertinib | — | PO / qd to end | 29 | 0-28 | 28 |
| | | Vehicle | | | | | |
| 2 | 10 | Zongertinib | 20 | PO / qd to end | 32 | 0-31 | 31 |
| 3 | 10 | Adagrasib | 100 | PO / qd to end | 67 | 0-66 | 66 |
| 4 | 10 | Zongertinib | 20 | PO / qd to end | 67 | 0-66 | 66 |
| | | Adagrasib | 100 | | | | |
| 5 | 3 | No Treatment | — | — | — | — | 0 |

Note:
Group 5 animals are used for the purpose of baseline collections and are excluded from weekly data capture.

### 2.1.2 PDX

ST524 is a patient derived colorectal carcinoma model that expresses a KRAS^{G12C} mutation.

### 2.1.3 Mice

Mice are treated as follows:

| Animal Supplier: | Charles River Laboratories | Feeding/Drinking Time: | Ad Libitum | |
|---|---|---|---|---|
| Acclimatization Period: | >24 Hours | Room Temperature: | 70-74° | F. |
| Identification: | Ear Notch/Lab Stamp | Relative Humidity: | 30-60% | |
| Caging Type: | Sealsafe ® Plus Techniplast, USA | Light Period: | 12 | Hours |

| | | | |
|---|---|---|---|
| Environment Enrichment: | Corncob bedding, nesting sheets, plastic | Data Capture Method: | Direct Electronic |
| Feed Type: | Teklad 2919 (Irradiated) 19% protein, 9% fat, 4% | Data Capture Instrument(s): | Digital Scale/Digital Caliper |
| Drinking Water: | Reverse Osmosis, 2 ppm Cl₂ | Data Storage Method: | Redundant Cloud Server |

### 2.1.4 Methodology

Tumor fragments from evaluated models are harvested from host animals and implanted into immune-deficient mice and studies are initiated at a mean tumor volume of approximately 175-300 mm³.

| | | |
|---|---|---|
| Description | Abbreviatio | Definition |

General

| | | |
|---|---|---|
| Body Weight | BW | Collected in grams, standard deviation reported |
| Tumor Volume | TV | Reported in mm³, Mean ± SD used; Formula: width² x length x |
| Tumor doubling time | Td | Log-linear growth of control group; Formula: Td = Log₁₀2/m |
| Route Of Administration | ROA | Drug introduction route: oral (PO), intraperitoneal (IP), |
| | | intravenous (IV), subcutaneous (SC), intratumoral (IT) |
| TGI Study | TGI | Study type where all groups end once the control reaches a |
| | | designated mean TV or timepoint. Stats based on delta TV vs. |
| | | Day 0 TV: |
| Group TGI study | gTGI | Study type where each group is ended once it reaches a designated |
| | | mean TV or timepoint. Stats based on delta TV vs. Day 0 TV; >3 |
| | | groups = ANOVA + Dunnett's, <3 groups = Student T-test |
| TGD Study | TGD | Study type where each individual animal is ended once it reaches |
| | | a designated TV or timepoint. Stats based on individual TTE; |
| | | Student T-test or Log-rank. |
| N = 1: Defined Study Enrollment | N = 1: DSE | Animals enrolled on M, Th up to three timepoints; a M or Th |
| | | holiday during enrollment period shifts to T, F for that protocol |
| | | only |
| To Be Determined | TBD | Information provided by Sponsor or START prior to initiation |

Weights and Tolerability

| | | |
|---|---|---|
| Percent Weight Change versus Day 0 | % vD₀ | Weight change versus a study initiation (Day 0) measurement |
| Weight Nadir; Maximum Mean % vD₀ | % vD₀₍ₘₐₓ₎ | Maximum weight loss versus a study initiation (Day 0) |
| Maximum Tolerated Dose | MTD | Drug concentration on evaluated schedule where >20% weight |
| | | loss for 7 or more days and/or 20% death or morbidity |
| Drug related animal death | D | Death as a result of agent toxicity |
| Technical related animal death | T | Death as a result of technician error |
| Tumor (Burden) related animal death | B | Death as a result of tumor-related weight loss or cachexia |
| Unknown related animal death | U | Cause of death cannot be determined |

Efficacy and Reporting

| | | |
|---|---|---|
| Tumor Growth Inhibition | % TGI | Percent mean tumor growth versus Day 0 between treatment (TX) |
| | | and control (C) groups; Formula: % TGI = 1 − (TXf_{avg} − TXi_{avg})/ |
| (Median) Time to Endpoint | M(TTE) | (Median) timepoint in days for an individual mouse or group to |
| | | reach a designated tumor volume or time endpoint. Mean ± SD |
| Tumor Growth Delay | T-C | Difference in days between MTTE of treatment and control |
| | | groups |
| Percent Tumor Regression | % TR | Percentage reduction in Day 0 tumor volume calculated at study |
| | | endpoint; mean value used if multiple regressions |
| Partial Responder | PR | TV ≥50% regression versus Day 0 for two consecutive |
| | | measurements over a period of ≥7 days during or at study |
| | | completion |
| Complete Responder | CR | TV <15 mm³ for two consecutive measurements over a period of |
| | | ≥7 days during or at study completion |
| Tumor Free Survivor | TFS | Durable CR at study completion; TV data excluded from TGI |
| | | calculations but not TGI stats; final study day assigned for TGD |
| | | stats |
| Long Term Survivor | LTS | Used in TGD efficacy studies to denote animals individual TV < |
| | | volume endpoint at study time endpoint. |

Samples

| | | |
|---|---|---|
| Flash Frozen | N | Tumor fragment (5x5+) flash frozen in LN₂ and stored at −80° C. |
| Formalin-Fixed, Paraffin Embedded | FFPE | Tumor fragment (5x5+) in 10% NBF for 48-72 H and then |
| | | embedded into a paraffin block |
| RNAlater | R | Tumor fragment (5x5) flash frozen in RNA solution and stored |
| | | at −80° C. |
| Plasma (K₂EDTA, K₃EDTA, or Li-Hep) | L | Whole blood: 1700 RCF 10′ @, 4° C., flash frozen stored at −80° C. |
| Serum (SST or Z) | S | Whole blood: RT 30′ 2000 RCF 10′ @ RT, flash frozen stored |
| | | at −80° C. |

### 2.1.5 Administration of Test Compounds

Zongertinib is suspended in Natrosol (0.5%) at a concentration of 2 mg/mL and adagrasib in Natrosol (0.5%) added with 1% DMSO at a concentration of 10 mg/mL. Compounds are administered with a volume of 10 ml/kg body weight.

Mice in the control group are treated with the vehicle of the zongertinib formulation, administered orally.

### 2.2. Results

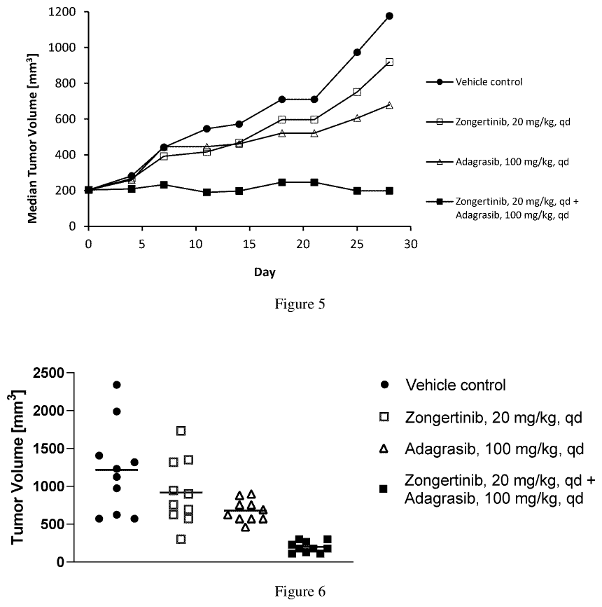

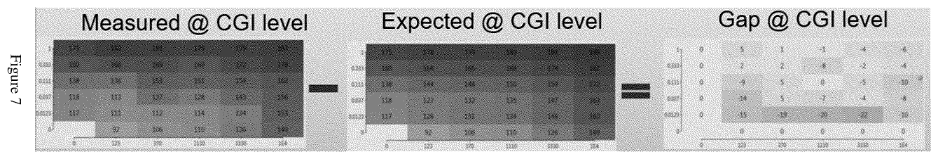

Results are reported in FIGS. 5 and 6. A combination of zongertinib and adagrasib shrank tumors whereas neither agent alone achieved this (FIG. 5).

Zongertinib and adagrasib evaluated alone and in combination were well tolerated with a maximum mean weight loss of 6% in any group and no drug-related deaths. Combination of zongertinib and adagrasib was active (p<0.0001).

It was demonstrated that a combination of zongertinib with adagrasib induces persistent tumor shrinkage in a PDX model whereas neither alone can.

### Example 3) Combination of Zongertinib with Sotorasib or Adagrasib in NSCLC Cell Lines

### 3.1 Materials & Methods

### 3.1.1 Assay Description

The CellTiter-Glo 2.0 Assay (Promega, #G9243) provides a homogeneous method to determine the number of viable cells in culture by quantitating the amount of ATP present, which indicates the presence of metabolically active cells. Addition of CellTiter-Glo Luminescent Cell Viability Reagent results in cell lysis and generation of a luminescent signal that is proportional to the amount of ATP present. The amount of ATP is directly proportional to the number of cells present in culture.

### 3.1.2 Assay Conditions

Numbers of cells per well indicated in Table 5 are seeded in white, sterile CulturePlate-96 (white with white bottom, PerkinElmer, #6005680). In general, cells seeded on plates are incubated overnight in a humidified incubator at 37° C. and 5% CO₂.

TABLE 6
Cell culture conditions for the CellTiter-Glo Luminescent Cell Viability Assay

| | Number of | | |
|---|---|---|---|
| | cells per | | |
| Name | well | Supplier | Culture media |
| NCI-H358 | 4000 | ATCC- | RPMI 1640 ATCC-formulated (Gibco, #A10491-01) or |
| | | CRL-5807 | RPMI 1640 ATCC-formulated (PAN Biotech, #P04- |
| | | | 18047) + 10% FBS (Gibco, #26140079) |
| NCI-H2122 | 4000 | ATCC | RPMI 1640 ATCC-formulated (Gibco, #A10491-01) or |
| | | CRL-5985 | RPMI 1640 ATCC-formulated (PAN Biotech, #P04- |
| | | | 18047) + 10% FBS (Gibco, #26140079) |

w3.1.3 Addition of test compounds and measurement

Compound start concentration and dilution steps are shown in Table 7.

TABLE 7
Start concentration and dilution steps for the
CellTiter-Glo Luminescent Cell Viability Assay

| | | NCI-H358 | | NCI-H2122 | |
|---|---|---|---|---|---|
| Zongertinib | start concentration | 1 | μM | 1 | μM |
| | dilution steps | 1:4 dilution | | 1:4 dilution | |
| Tucatinib | start concentration | 1 | μM | 1 | μM |
| | dilution steps | 1:4 dilution | | 1:4 dilution | |
| Sotorasib | start concentration | 0.1 | μM | 1 | μM |
| | dilution steps | 1:4 dilution | | 1:4 dilution | |
| Adagrasib | start concentration | 0.1 | μM | 1 | μM |

| | | |
|---|---|---|
| dilution steps | 1:4 dilution | 1:4 dilution |

Compounds (small molecules inhibitors of HER2 or KRAS^{G12C}; see the table above for detailed information) or DMSO are added to the cells in 4-fold serial dilution (highest test concentration see table 7) in duplicates.

At the time of test compound addition, a “time zero” (t=0) untreated cell plate is measured upon addition of CellTiter-Glo® Luminescent Cell Viability Reagent (Promega, CatNo. G9243). The plates are incubated for 15 minutes at room temperature to induce cell lysis and to stabilize the luminescent signal.

The luminescent signal of each well was measured in an EnSpire or EnSight Multilabel Plate Reader (PerkinElmer).

After 5 days of incubation with test compounds, the cells of all other plates are measured upon addition of CellTiter-Glo Luminescent Cell Viability Reagent (Promega, CatNo. G9243) to assess cell viability.

The plates are incubated for 15 minutes at room temperature to induce cell lysis and to stabilize the luminescent signal. The luminescent signal of each well was measured in an EnSpire or EnSight Multilabel Plate Reader (PerkinElmer).

### 3.1.4 Data Analysis

The data can be analyzed with the Boehringer Ingelheim proprietary software MegaLab (curve fitting based on the program PRISM, GraphPad Inc.).

The CellTiter-Glo® assay output for vehicle-treated control cells after 5 days of incubation, corresponding to 100% cell viability, is taken as the reference signal for all subsequent calculations. Relative cell viability in compound-treated cultures (signal percent of control, “POC”) can be calculated according to the following formula:

$${POC}_{( t = 120 h )} = 100 \times \frac{{luminescence}_{( compound wells )}}{{luminescence}_{( control wells )}}$$

In addition, for each compound-treated culture, the luminescent signal after incubation for 120 hours (POC (t=120 h)) can be related to the signal at the start of treatment (POC (t=0 h)) according to the following formula:

$${POC}_{( t = 0 h )} = 100 \times \frac{luminescence at t = 0 {h}_{( control wells )}}{luminescence at t = 120 {h}_{( control wells )}}$$

To calculate concentration-response curves, the POC data are analyzed using a four-parameter log-logistic function without any upper or lower limitation.

Relative cell growth inhibition (CGI %) in compound-treated cultures can be calculated according to the following formula:

$$\% {CGI}^{120 h} = - [ \begin{matrix}{S}_{t}^{120} \geq {S}_{c}^{0} : [ 1 - \frac{{S}_{t}^{120 h} - {S}_{c}^{0 h}}{{S}_{c}^{120 h} - {S}_{c}^{0 h}} ] \times 100 \% \\ {S}_{t}^{120} < {S}_{c}^{0} : [ 1 - \frac{{S}_{t}^{120 h} - {S}_{c}^{0 h}}{{S}_{c}^{0 h}} ] \times 100 \%\end{matrix}$$

Sₜ¹²⁰=POC_{(t=120 h)}
S_{c}⁰=POC_{(t=0 h)}

A CGI of >0% and <100% reflects a partial growth-inhibitory effect relative to vehicle-treated controls, a CGI of 100% is equivalent of complete blockade of growth, and a CGI of >100% is indicative of net cell death.

Calculation of BLISS CGI GAP using Boehringer Ingelheim proprietary software MegaLab: Bliss Independence Model was published by Bliss (BLISS, 1939).

The model describes the effect of drug A and of drug B and the combination of both and asks whether the combined effect is different from expected, whereas
Eₐ=effect of drug A at dose a
E_{b}=effect of drug B at dose b
E_{a,b}=combined effects of drugs A and B at doses a and b, respectively

$${E}_{a , b , expected} = {E}_{a} + {E}_{b} ( 1 - {E}_{a} ) = {E}_{a} + {E}_{b} - {E}_{a} \times {E}_{b}$$

$$BLISS GAP = {E}_{a , b , measured} - {E}_{a , b , expected}$$

The assumption is that both drugs act independently and Eₐ and E_{b} are scaled to be in between 0, and 100 POC and then translated in CGI based on the formula above. Measurement CGI, expected CGI and their difference resulting in BLISS GAP CGI are illustrated in FIG. 7.

### 3.2 Results

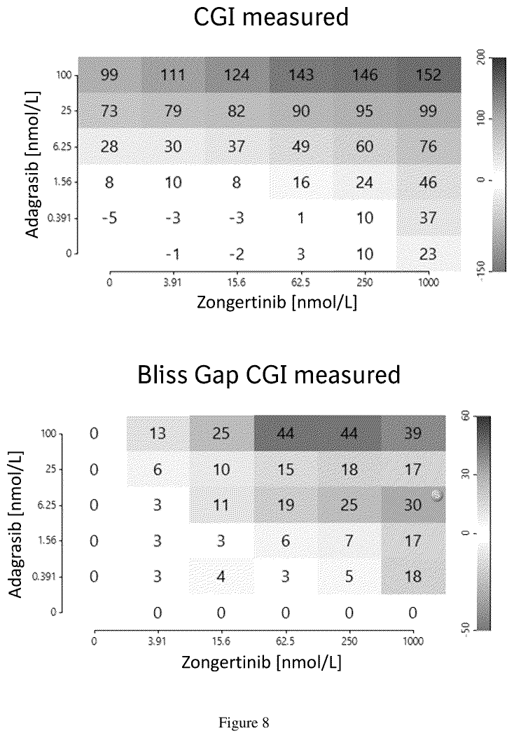

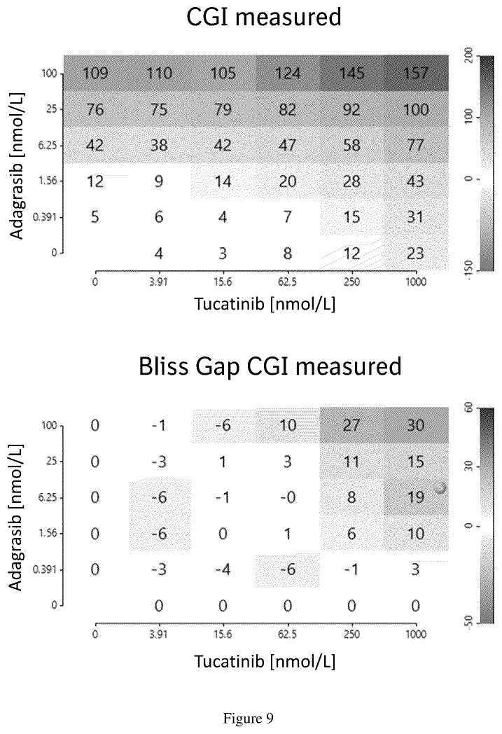

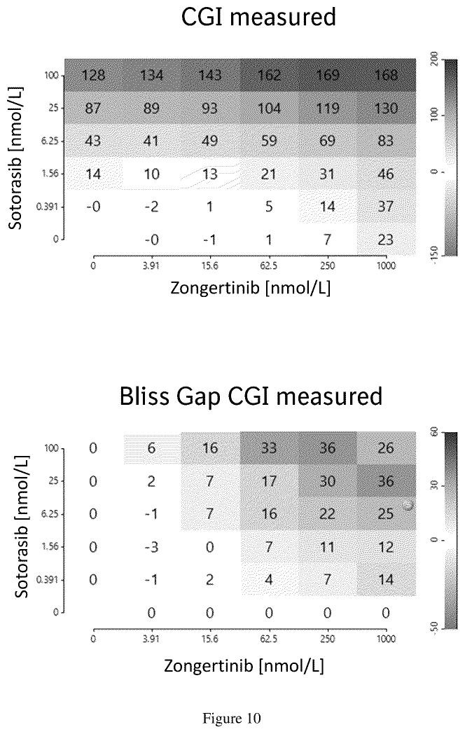

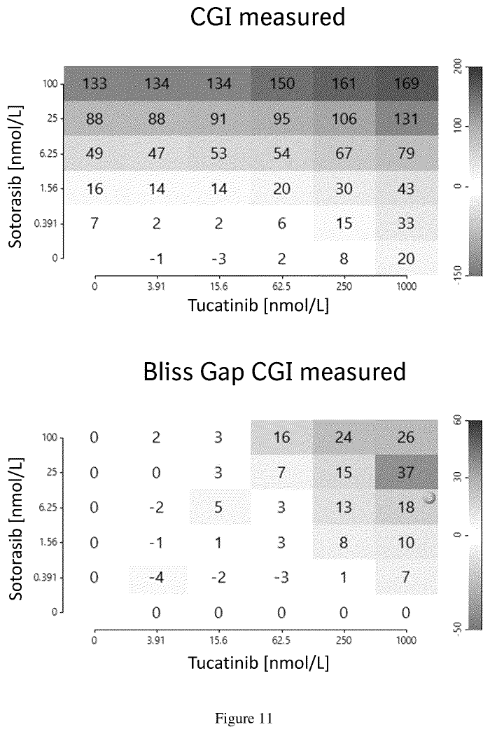

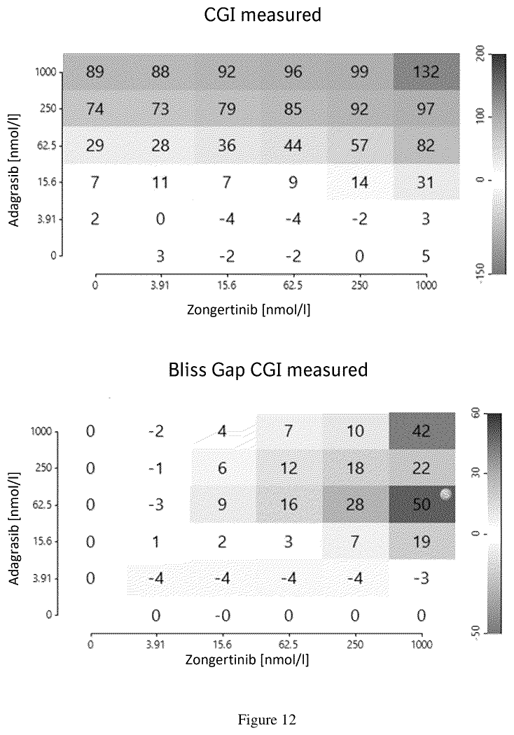

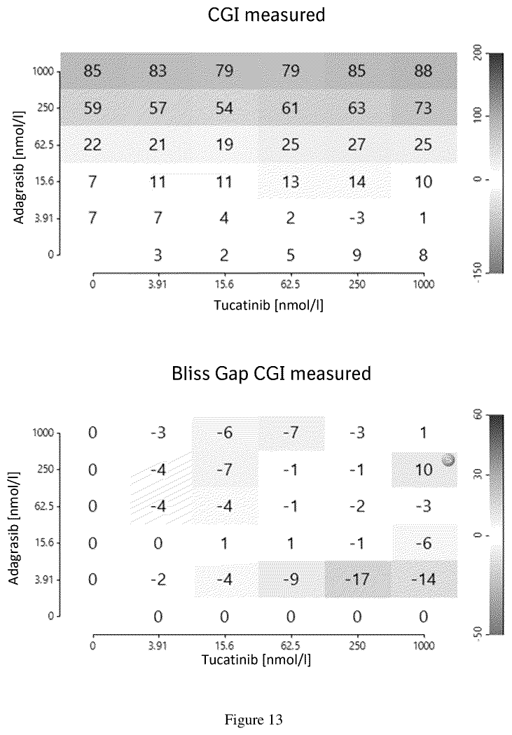

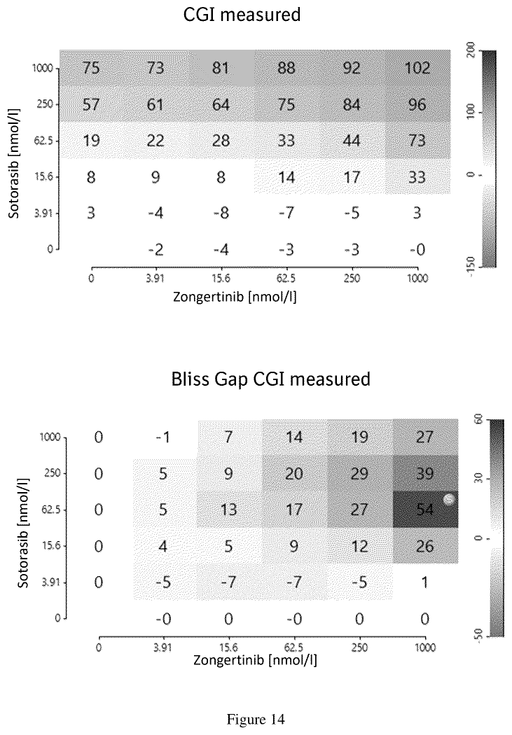

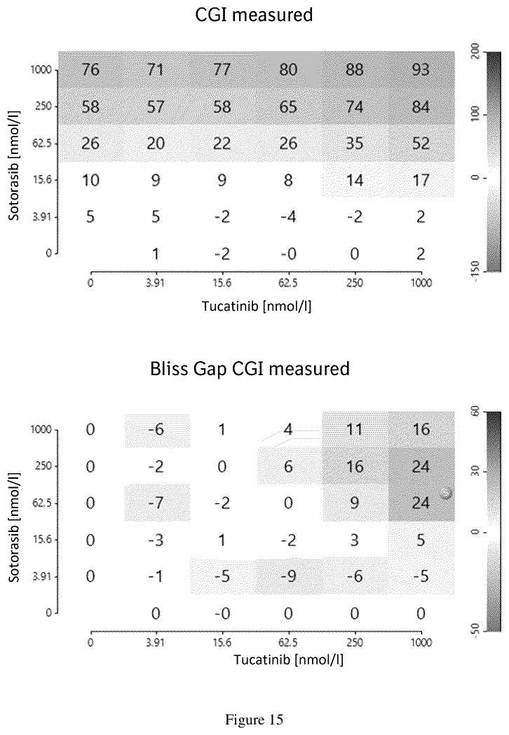

### Results are Reported in FIGS. 8-15.

The anti-proliferative activity of zongertinib in combination with KRAS^{G12C} inhibitors, sotorasib or adagrasib, was tested in dose-titration experiments on two human KRAS^{G12C}-mutant lung cancer cell lines, NCI-H358 and NCI-H2122. These cell lines are ERBB2 wild type.

Within a certain concentration range the data for NCI-H358 and NCI-H2122 show combined, synergistic anti-proliferative activity of zongertinib and either sotorasib or adagrasib, as seen by increasing cell growth inhibition (CGI) values and Bliss values.

Tucatinib in combination with either KRAS^{G12C} inhibitor, sotorasib or adagrasib, show also combined, synergistic anti-proliferative activity on these two cell lines, NCI-H358 or NCI-H2122, however the combined effect is less pronounced compared to the data shown for zongertinib under these test conditions.

These data show for NCI-H358 and NCI-H2122 that zongertinib in combination with either KRAS^{G12C} inhibitor has enhanced, synergistic anti-proliferative activity and might differentiate from tucatinib in combination with either KRAS^{G12C} inhibitor, which was less efficacious.

### Example 4) Combination of Zongertinib and Adagrasib in Adagrasib Resistant KRAS G12C Mutant NSCLC Tumor Models

### 4.1 Materials & Methods

### Generation of Adagrasib Resistant Tumor Models

Both parental KRAS G12C mutant NSCLC tumor models (TC314 and TC807) are treated with adagrasib at 100 mg/kg QD 5on/2off continuously until tumor outgrowth. After tumors reach ˜1,500 mm³, they are expanded into 20 mice. 14 days after implantation surgery (wound clip coming off), mice are randomized to receive vehicle or adagrasib (100 mg/kg QD 5on/2off) to confirm resistance. All resistant tumors while passaging/growth are mostly under adagrasib treatment to keep selection pressure.

### Efficacy Studies

For efficacy studies, ONE single large tumor (on adagrasib treatment) is expanded into N=60 mice. 14 days after the surgery, all mice start receiving adagrasib at 100 mg/kg QD 5on/2off until tumors reach 150-250 mm³. The mice are randomized to 3 groups receiving vehicle, adagrasib alone, or adagrasib+zongertinib. Unfortunately, resistance tumors often grow with high variation under selection pressure. The planned zongertinib single agent group had to be sacrificed to achieve a higher number for the remaining planned groups.

For the TC314R model the treatment groups are:
A) 0.1% Natrosol 5on/2off (n=6 mice);
B) 100 mg/kg adagrasib/MRTX849 5on/2off (n=6 mice);
C) 100 mg/kg adagrasib/MRTX849 5on/2off and 20 mg/kg zongertinib 5on/2off (n=6 mice).

For the TC807R model the treatment groups are:
A) 0.5% Natrosol (n=5 mice)
B) 100 mg/kg QD adagrasib/MRTX849 (n=5 mice)
D) 100 mg/kg QD adagrasib/MRTX849 and 20 mg/kg QD zongertinib (n=6 mice).

### 4.2 Results

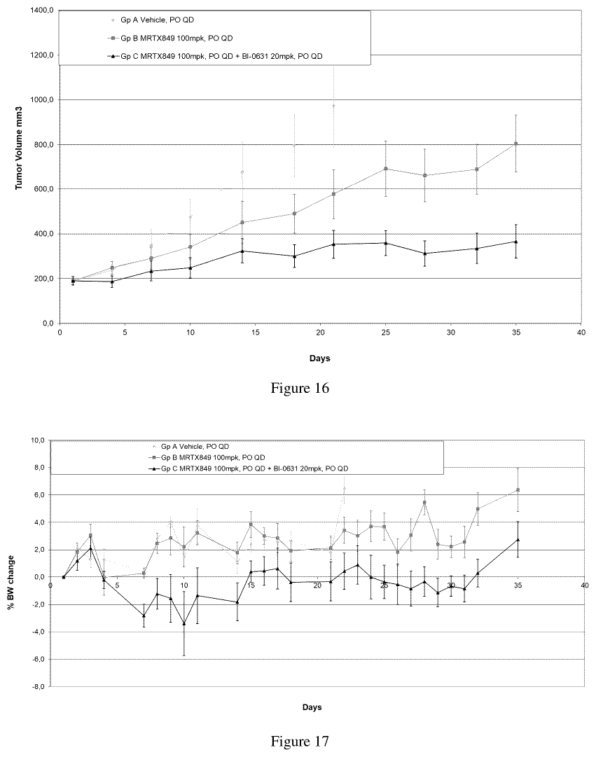

Results for the TC314R model are shown in FIGS. 16 and 17. Whereas 100 mg/kg 5on/2off adagrasib/MRTX849 (group B, middle curve in FIG. 16) did delay tumor growth to a certain extent, the combination of 100 mg/kg 5on/2off adagrasib/MRTX849 and 20 5on/2off mg/kg zongertinib (group C, bottom curve in FIG. 16) did control the tumor outgrowth. As judged by body weight changes, the treatment was tolerated (FIG. 17).

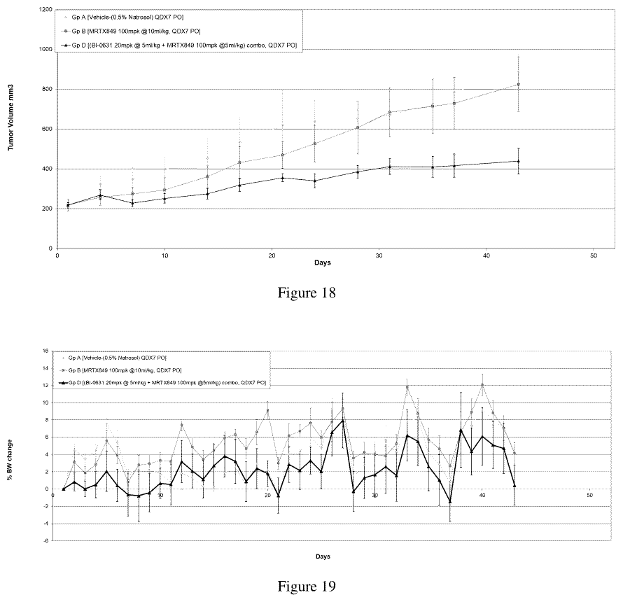

Results for the TC807R model are shown in FIGS. 18 and 19. Whereas 100 mg/kg QD adagrasib/MRTX849 (group B, middle curve with regards to the last point in FIG. 18) did not control tumor growth the combination of 100 mg/kg QD adagrasib/MRTX849 and 20 QD mg/kg zongertinib (group C, bottom curve in FIG. 18) did delay the tumor outgrowth. As judged by body weight changes the treatment was well tolerated (FIG. 19).

### Example 5) Combination of Zongertinib and Adagrasib in Xenografts Derived from Patients with KRAS^{G12C}-Mutant Lung Cancer

In this Example, the anti-tumor activity of zongertinib in combination with the KRAS^{G12C} inhibitor adagrasib is evaluated in a subcutaneous xenograft mouse model derived from the KRAS^{G12C}-mutant lung cancer in NMRI-Foxn1^{nu} mice.

### 5.1 Materials & Methods

The results reported below can be obtained following a procedure similar to that of Example 2, except that ST4341 is used instead of ST524. ST524 is a patient derived lung cancer model that expresses and is dependent on a KRAS^{G12C} mutation but is HER2 independent.

### 5.2 Results

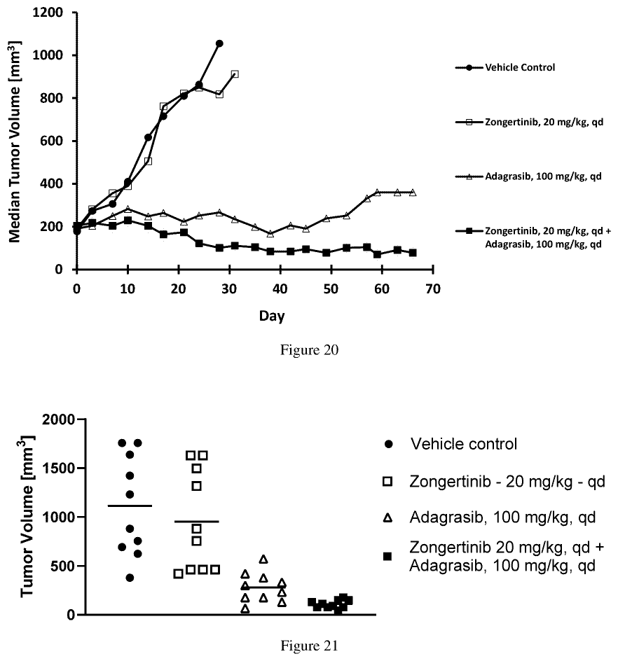

### Results are Reported in FIGS. 20 and 21.

Adagrasib achieved tumor stasis in the lung cancer ST-4341 KRAS^{G12C} PDX model, with signs of outgrowth beyond day 50. The combination treatment of zongertinib and adagrasib deepened the response and achieved tumor regression beyond day 60 (FIG. 20). Overall, these data suggest a synergistic effect for combined HER2 and KRAS^{G12C} inhibition.

1.-14. (canceled)

15. A method of treating cancer, the method comprising administering to a patient in need thereof a therapeutically effective amount of a combination comprising:
zongertinib or a pharmaceutically acceptable salt thereof, and
a KRAS G12C inhibitor.

16. The method according to claim 15, wherein the KRAS G12C inhibitor is selected from the group consisting of: sotorasib, adagrasib, compounds Ib-1 to Ib-16, Ic-1 to Ic-9, Id-1 to Id-9 and Ie-1 of WO2021/245051, compounds Ia-1 to Ia-170 of WO2021/245055, compounds Ia-1 to Ia-4 and Ib-1 to Ib-9 of WO2023/099612, divarasib, opnurasib, garsorasib, glecirasib, IBI351, RMC-6291, LY3537982, JNJ-74699157 and LY3499446.

17. The method according to claim 15, wherein the KRAS G12C inhibitor is sotorasib or adagrasib.

18. The method according to claim 15, wherein the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

19. The method according to claim 15, wherein the cancer is HER2 overexpressed, HER2 amplified and/or HER2 mutant.

20. The method according to claim 15, wherein the cancer is KRAS G12C mutant.

21. The method according to claim 15, wherein the cancer is resistant to treatment with the KRAS G12C inhibitor.

22. The method according to claim 15, wherein the combination produces a synergistic therapeutic effect as compared to the sole administration of zongertinib or the pharmaceutically acceptable salt thereof or of the sole administration of the KRAS G12C inhibitor.

23. The method according to claim 15, wherein the zongertinib or pharmaceutically acceptable salt thereof, and the KRAS G12C inhibitor are administered concurrently.

24. A kit comprising:
(i) a first pharmaceutical composition comprising a therapeutically effective amount of zongertinib or a pharmaceutically acceptable salt thereof and a pharmaceutically acceptable excipient; and
(ii) a second pharmaceutical composition comprising a therapeutically effective amount of a KRAS G12C inhibitor and a pharmaceutically acceptable excipient; and wherein the first and second pharmaceutical compositions are packaged together in a single container.

25. The kit according to claim 24, wherein the KRAS G12C inhibitor is selected from the group consisting of: sotorasib, adagrasib, compounds Ib-1 to Ib-16, Ic-1 to Ic-9, Id-1 to Id-9 and Ie-1 of WO2021/245051, compounds Ia-1 to Ia-170 of WO2021/245055, compounds Ia-1 to Ia-4 and Ib-1 to Ib-9 of WO2023/099612, divarasib, opnurasib, garsorasib, glecirasib, IB1351, RMC-6291, LY3537982, JNJ-74699157 and LY3499446.

26. The kit according to claim 24, wherein the KRAS G12C inhibitor is sotorasib or adagrasib.

27. The kit according to claim 24, further comprising instructions for administering the first and second pharmaceutical compositions concurrently.

28. The kit according to claim 24, further comprising instructions specifying a type of cancer to treat and wherein the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

29. The method according to claim 15, further comprising instructions specifying a type of cancer to treat and wherein the cancer is HER2 overexpressed, HER2 amplified and/or HER2 mutant.

30. The method according to claim 15, further comprising instructions specifying a type of cancer to treat and wherein the cancer is KRAS G12C mutant.

31. The method according to claim 15, further comprising instructions specifying a type of cancer to treat and wherein the cancer is resistant to treatment with the KRAS G12C inhibitor.

32. The method according to claim 15, further comprising instructions specifying at least one dosage regimen that produces a synergistic therapeutic effect as compared to the sole administration of zongertinib or the pharmaceutically acceptable salt thereof or to the sole administration of the KRAS G12C inhibitor.

33. The method according to claim 15, wherein the zongertinib or pharmaceutically acceptable salt thereof, and the KRAS G12C inhibitor are administered concurrently.
